SEQUENCE LISTING (84 sequences)

SEQ ID NO: 1 - DNA, 20 bp, Artificial Sequence
  20 nucleotide DNA sequence encoding the Csy4 site
gttcactgccgtataggcag

SEQ ID NO: 2 - DNA, 28 bp, Artificial Sequence
  28 nucleotide DNA sequence encoding the Csy4 site
gttcactgccgtataggcagctaagaaa

SEQ ID NO: 3 - RNA, 20 nt, Artificial Sequence
  20 Csy4 RNA sequence
guucacugccguauaggcag

SEQ ID NO: 4 - RNA, 28 nt, Artificial Sequence
  28 Csy4 RNA sequence
guucacugccguauaggcagcuaagaaa

SEQ ID NO: 5 - DNA, 77 bp, Artificial Sequence
  pre-tRNAGly
aacaaagcaccagtggtctagtggtagaatagtaccctgccacggtacagacccgggttcgattcccggctggtgca

SEQ ID NO: 6 - DNA, 75 bp, Danio rerio
  Dr-tRNAGly(GCC)
gtgagcattggtggttcagtggtagaattctcgcctgccacgcgggaggcccgggttcgattcccggccaatgca

SEQ ID NO: 7 - DNA, 83 bp, Danio rerio
  Dr-tRNALys(CTT)
gttctcatcagcccggctagctcagtcggtagagcatgagactcttaatctcagggtcgtgggttcgagccccacgtcgggcg

SEQ ID NO: 8 - DNA, 81 bp, Danio rerio
  Dr-tRNAAsn(GTT)
gctatctgtctctgtggcgcaatcggttagcgcgttcggctgttaaccgaaaggttggtggttcgagcccacccagggacg

SEQ ID NO: 9 - DNA, 81 bp, Danio rerio
  Dr-tRNAMet(CAT)
gcctgaaggtttccgtagtgtagtggttatcacgttcgcctcatacgcgaaaggtccccagttcgaaactgggcggaaaca

SEQ ID NO: 10 - DNA, 79 bp, Danio rerio
  Dr-tRNAGln(CTG)
gacttgaggttccatggtgtaatggttagcactctggactctgaatccagcgatccgagttcaaatctcggtgggacca

SEQ ID NO: 11 - DNA, 89 bp, Danio rerio
  Dr-tRNASer(GCT)
ggaaaatgacgaggtggccgagtggttaaggcgatggactgctaatccattgtgctttgcacgcatgggttcgaatcccatcctcgtcg

SEQ ID NO: 12 - DNA, 79 bp, Danio rerio
  Dr-tRNAThr(AGT)
gcagcggcgccgtggcttagttggttaaagcgcctgtctagtaaacaggagatcctgggttcgaatcccagcggtgcct

SEQ ID NO: 13 - DNA, 76 bp, Danio rerio
  Dr-tRNAHis(GTG)
gctcgccgtgatcgtacagtggttagtactctgcgttgtggccgcagcaaccccggttcgaatccgggtcacggca

SEQ ID NO: 14 - DNA, 87 bp, Danio rerio
  Dr-tRNALeu(CAG)
gcatgtcaggatggccgagtggtctaaggcgctgcgttcaggtcgcagtctcccctggaggcgtgggttcgaatcccacttctgaca

SEQ ID NO: 15 - DNA, 78 bp, Oryza sativa
  Os-tRNAGly(GCC) Shiraki and Kawakami 2
gaacaaagcaccagtggtctagtggtagaatagtaccctgccacggtacagacccgggttcgattcccggctggtgca

SEQ ID NO: 16 - DNA, 78 bp, Artificial Sequence
  Os-tRNAGly(GCC)-scrambled
gaacctcttacacgcgcagatcaactaaatgtacactgcgacggtccgtggctccgagaggggttacagggtacgctg

SEQ ID NO: 17 - DNA, 75 bp, Artificial Sequence
  Dr-tRNAGly(GCC)-scrambled
gcgctgtggcgtaccgggtacgtactcgcttgactgggttggtactaggcgaaaccagctccgtgggattgcacc

SEQ ID NO: 18 - DNA, 44 bp, Artificial Sequence
  Hammerhead ribozyme (HH)
gttccccctgatgagtccgtgaggacgaaacgagtaagctcgtc

SEQ ID NO: 19 - DNA, 68 bp, Hepatitis delta virus
  Hepatitis delta virus ribozyme (HDV)
ggccggcatggtcccagcctcctcgctggcgccggctgggcaacatgcttcggcatggcgaatgggac

SEQ ID NO: 20 - DNA, 962 bp, Oryza sativa
  Intron sequence
gtacgctgcttctcctctcctcgcttcgtttcgattcgatttcggacgggtgaggttgttttgttgctagatccgattggtggttagggttgtcgatgtgattatcgtgagatgtttaggggttgtagatctgatggttgtgatttgggcacggttggttcgataggtggaatcgtggttaggttttgggattggatgttggttctgatgattggggggaatttttacggttagatgaattgttggatgattcgattggggaaatcggtgtagatctgttggggaattgtggaactagtcatgcctgagtgattggtgcgatttgtagcgtgttccatcttgtaggccttgttgcgagcatgttcagatctactgttccgctcttgattgagttattggtgccatgggttggtgcaaacacaggctttaatatgttatatctgttttgtgtttgatgtagatctgtagggtagttcttcttagacatggttcaattatgtagcttgtgcgtttcgatttgatttcatatgttcacagattagataatgatgaactcttttaattaattgtcaatggtaaataggaagtcttgtcgctatatctgtcataatgatctcatgttactatctgccagtaatttatgctaagaactatattagaatatcatgttacaatctgtagtaatatcatgttacaatctgtagttcatctatataatctattgtggtaatttctttttactatctgtgtgaagattattgccactagttcattctacttatttctgaagttcaggatacgtgtgctgttactacctatctgaatacatgtgtgatgtgcctgttactatctttttgaatacatgtatgttctgttggaatatgtttgctgtttgatccgttgttgtgtccttaatcttgtgctagttcttaccctatctgtttggtgattatttcttgcag

SEQ ID NO: 21 - RNA, 77 nt, Caenorhabditis elegans
  Example miRNA sequence
ccggccuguucccugagaccucaagugugaguguacuauugaugcuucacaccugggcucuccggguaccaggacgg

SEQ ID NO: 22 - DNA, 146 bp, Homo sapiens
  Example snoRNA sequence
cttcagtgatgacacgatgacgagtcagaaaggtcacgtcctgctcttggtccttgtcagtgccatgttctgtggtgctgtgcacgagttcctttggcagaagtgtcctatttattgatcgatttagaggcatttgtctgagaagg

SEQ ID NO: 23 - DNA, 53 bp, Artificial Sequence
  Forward Primer with Overhang, Where N denotes a gRNA Target sequence
  variation 1..20: /standard_name=“Where n is any base selected from a, c, g, t”
nnnnnnnnnnnnnnnnnnnngttttagagctagaaatagcaagttaaaataag

SEQ ID NO: 24 - DNA, 20 bp, Artificial Sequence
  Reverse Primer with Overhang, where Phos denotes a phosphate group
  misc_feature 1: /standard_name=“5′-phosphorylation of c”
ctgcctatacggcagtgaac

SEQ ID NO: 25 - DNA, 20 bp, Artificial Sequence
  Forward Primer for sequencing of fragments after Round 1 PCR and isolation
ctcacatgttctttcctgcg

SEQ ID NO: 26 - DNA, 35 bp, Artificial Sequence
  Forward primer
gcatcgtctcatgccgttcactgccgtataggcag

SEQ ID NO: 27 - DNA, 33 bp, Artificial Sequence
  Reverse primer
atgccgtctcatagtaaaagcaccgactcggtg

SEQ ID NO: 28 - DNA, 35 bp, Artificial Sequence
  Forward primer
gcatcgtctcaactagttcactgccgtataggcag

SEQ ID NO: 29 - DNA, 33 bp, Artificial Sequence
  Reverse primer
atgccgtctcatctgaaaagcaccgactcggtg

SEQ ID NO: 30 - DNA, 35 bp, Artificial Sequence
  Forward primer
gcatcgtctcacagagttcactgccgtataggcag

SEQ ID NO: 31 - DNA, 33 bp, Artificial Sequence
  Reverse primer
atgccgtctcagtaaaaaagcaccgactcggtg

SEQ ID NO: 32 - DNA, 35 bp, Artificial Sequence
  Forward primer
gcatcgtctcattacgttcactgccgtataggcag

SEQ ID NO: 33 - DNA, 33 bp, Artificial Sequence
  Reverse primer
atgccgtctcacacaaaaagcaccgactcggtg

SEQ ID NO: 34 - DNA, 35 bp, Artificial Sequence
  Forward primer
gcatcgtctcatgtggttcactgccgtataggcag

SEQ ID NO: 35 - DNA, 33 bp, Artificial Sequence
  Reverse primer
atgccgtctcagctcaaaagcaccgactcggtg

SEQ ID NO: 36 - DNA, 35 bp, Artificial Sequence
  Forward primer
gcatcgtctcagagcgttcactgccgtataggcag

SEQ ID NO: 37 - DNA, 33 bp, Artificial Sequence
  Reverse primer
atgccgtctcagaataaaagcaccgactcggtg

SEQ ID NO: 38 - DNA, 35 bp, Artificial Sequence
  Forward primer
gcatcgtctcaattcgttcactgccgtataggcag

SEQ ID NO: 39 - DNA, 33 bp, Artificial Sequence
  Reverse primer
atgccgtctcattcgaaaagcaccgactcggtg

SEQ ID NO: 40 - DNA, 35 bp, Artificial Sequence
  Forward primer
gcatcgtctcacgaagttcactgccgtataggcag

SEQ ID NO: 41 - DNA, 33 bp, Artificial Sequence
  Reverse primer
atgccgtctcacggtaaaagcaccgactcggtg

SEQ ID NO: 42 - DNA, 35 bp, Artificial Sequence
  Forward primer
gcatcgtctcaaccggttcactgccgtataggcag

SEQ ID NO: 43 - DNA, 33 bp, Artificial Sequence
  Reverse primer
atgccgtctcaagttaaaagcaccgactcggtg

SEQ ID NO: 44 - DNA, 35 bp, Artificial Sequence
  Forward primer
gcatcgtctcaaactgttcactgccgtataggcag

SEQ ID NO: 45 - DNA, 33 bp, Artificial Sequence
  Reverse primer
atgccgtctcatcctaaaagcaccgactcggtg

SEQ ID NO: 46 - DNA, 35 bp, Artificial Sequence
  Forward primer
gcatcgtctcaaggagttcactgccgtataggcag

SEQ ID NO: 47 - DNA, 33 bp, Artificial Sequence
  Reverse primer
atgccgtctcattttaaaagcaccgactcggtg

SEQ ID NO: 48 - DNA, 35 bp, Artificial Sequence
  Forward primer
gcatcgtctcaaaaagttcactgccgtataggcag

SEQ ID NO: 49 - DNA, 33 bp, Artificial Sequence
  Reverse primer
atgccgtctcattgcaaaagcaccgactcggtg

SEQ ID NO: 50 - DNA, 24 bp, Artificial Sequence
  Forward Primer (binds pTDH3)
gacggtaggtattgattgtaattc

SEQ ID NO: 51 - DNA, 22 bp, Artificial Sequence
  Reverse Primer (binds tTDH1)
tgcttaatcttgtcttggctta

SEQ ID NO: 52 - DNA, 15 bp, Artificial Sequence
  Forward
gcatcgtctcatgcc

SEQ ID NO: 53 - DNA, 15 bp, Artificial Sequence
  Reverse
atgccgtctcatagt

SEQ ID NO: 54 - DNA, 15 bp, Artificial Sequence
  Forward
gcatcgtctcaacta

SEQ ID NO: 55 - DNA, 15 bp, Artificial Sequence
  Reverse
atgccgtctcatctg

SEQ ID NO: 56 - DNA, 15 bp, Artificial Sequence
  Forward
gcatcgtctcacaga

SEQ ID NO: 57 - DNA, 15 bp, Artificial Sequence
  Reverse
atgccgtctcagtaa

SEQ ID NO: 58 - DNA, 15 bp, Artificial Sequence
  Forward
gcatcgtctcattac

SEQ ID NO: 59 - DNA, 15 bp, Artificial Sequence
  Reverse
atgccgtctcacaca

SEQ ID NO: 60 - DNA, 15 bp, Artificial Sequence
  Forward
gcatcgtctcatgtg

SEQ ID NO: 61 - DNA, 15 bp, Artificial Sequence
  Reverse
atgccgtctcagctc

SEQ ID NO: 62 - DNA, 15 bp, Artificial Sequence
  Forward
gcatcgtctcagagc

SEQ ID NO: 63 - DNA, 15 bp, Artificial Sequence
  Reverse
atgccgtctcagaat

SEQ ID NO: 64 - DNA, 15 bp, Artificial Sequence
  Forward
gcatcgtctcaattc

SEQ ID NO: 65 - DNA, 15 bp, Artificial Sequence
  Reverse
atgccgtctcattcg

SEQ ID NO: 66 - DNA, 15 bp, Artificial Sequence
  Forward
gcatcgtctcacgaa

SEQ ID NO: 67 - DNA, 15 bp, Artificial Sequence
  Reverse
atgccgtctcacggt

SEQ ID NO: 68 - DNA, 15 bp, Artificial Sequence
  Forward
gcatcgtctcaaccg

SEQ ID NO: 69 - DNA, 15 bp, Artificial Sequence
  Reverse
atgccgtctcaagtt

SEQ ID NO: 70 - DNA, 15 bp, Artificial Sequence
  Forward
gcatcgtctcaaact

SEQ ID NO: 71 - DNA, 15 bp, Artificial Sequence
  Reverse
atgccgtctcatcct

SEQ ID NO: 72 - DNA, 15 bp, Artificial Sequence
  Forward
gcatcgtctcaagga

SEQ ID NO: 73 - DNA, 15 bp, Artificial Sequence
  Reverse
atgccgtctcatttt

SEQ ID NO: 74 - DNA, 15 bp, Artificial Sequence
  Forward
gcatcgtctcaaaaa

SEQ ID NO: 75 - DNA, 15 bp, Artificial Sequence
  Reverse
atgccgtctcattgc

SEQ ID NO: 76 - DNA, 2680 bp, Artificial Sequence
  Intermediate vector 1 (psl040-1st-acceptor vector for up to 6 grnas
aaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcagggggggccagcaacgcggcctttttacggttcctggccttttgctggccttttgctcacatgttctttcctgcgttatcccctgattctgtggataaccgtagggtctcattctctgccgagacggaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttatacgtctctatcctgcctgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtg

SEQ ID NO: 77 - DNA, 2680 bp, Artificial Sequence
  Intermediate vector 2 (psl040-2ndt-acceptor vector for up to 6 grnas
ctgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcagggggggccagcaacgcggcctttttacggttcctggccttttgctggccttttgctcacatgttctttcctgcgttatcccctgattctgtggataaccgtagggtctcatgccctgccgagacggaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttatacgtctctatccctaatgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttcca

SEQ ID NO: 78 - DNA, 2680 bp, Artificial Sequence
  Intermediate vector 3 (psl040-3rd-acceptor vector for up to 6 grnas
ctccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcagggggggccagcaacgcggcctttttacggttcctggccttttgctggccttttgctcacatgttctttcctgcgttatcccctgattctgtggataaccgtagggtctcactaactgccgagacggaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttatacgtctctatccaccatgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattca

SEQ ID NO: 79 - DNA, 2680 bp, Artificial Sequence
  Intermediate vector 4 (psl040-4th-acceptor vector for up to 6 grnas
gcacgagggagcttccagggggaaacgcctggtatctttatagtcctgtcgggtttcgccacctctgacttgagcgtcgatttttgtgatgctcgtcagggggggccagcaacgcggcctttttacggttcctggccttttgctggccttttgctcacatgttctttcctgcgttatcccctgattctgtggataaccgtagggtctcaaccactgccgagacggaaagtgaaacgtgatttcatgcgtcattttgaacattttgtaaatcttatttaataatgtgtgcggcaattcacatttaatttatgaatgttttcttaacatcgcggcaactcaagaaacggcaggttcggatcttagctactagagaaagaggagaaatactagatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggttccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaggacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgaccaggcatcaaataaaacgaaaggctcagtcgaaagactgggcctttcgttttatctgttgtttgtcggtgaacgctctctactagagtcacactggctcaccttcgggtgggcctttctgcgtttatacgtctctatccatcctgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagc

SEQ ID NO: 80 - DNA, 2351 bp, Artificial Sequence
  pLS039 - pTDH with TTC
ggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtagtcggtctcaaacgcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaagatcttcttgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttag

SEQ ID NO: 81 - DNA, 1915 bp, Artificial Sequence
  pLS070_-_tTDH1_[4]_modi
ttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtatcggtctcatgccgttcactgccgtataggcagctcgagataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacggctgtgagaccagaccaataaaaaacgcccggcggcaaccgagcgttctgaacaaatccagatggagttctgaggtcattactggatctatcaacaggagtccaagcgagctcgatatcaaa

SEQ ID NO: 82 - DNA, 1915 bp, Artificial Sequence
  pLS071_-_tTDH1_[4]_modi
aacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtatcggtctcactaagttcactgccgtataggcagctcgagataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacggctgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaa

SEQ ID NO: 83 - DNA, 1915 bp, Artificial Sequence
  pLS072_-_tTDH1_[4]_modi
taatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtatcggtctcaaccagttcactgccgtataggcagctcgagataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacggctgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgta

SEQ ID NO: 84 - DNA, 1915 bp, Artificial Sequence
  pLS073_-_tTDH1_[4]_modi
tccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgcccgatcaatcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtatcggtctcaatccgttcactgccgtataggcagctcgagataaagcaatcttgatgaggataatgatttttttttgaatatacataaatactaccgtttttctgctagattttgtgatgacgtaaataagtacatattactttttaagccaagacaagattaagcattaactttacccttttctttctaagtttcaatattagttatcactgtttaaaagttatggcgagaacgtcggcggttaaaatatattaccctgaacggctgtgagaccagaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgaaacgaaaaacatattctcaataaaccctttagggaaataggccaggttttcaccgtaacacgccacatcttgcgaatatatgtgtagaaactgccggaaatcgtcgtggtattcac

(19) United States
(12) Patent Application Publication
(10) Pub. No.: US 2021/0371859 A1
(43) Pub. Date: Dec. 2, 2021

(54) RNA MEDIATED GENE REGULATING METHODS

(71) Applicant: Imperial College of Science, Technology and Medicine, London (GB)

(72) Inventors: William Michael Shaw, London (GB); Rodrigo Ledesma Amaro, London (GB); Lucie Studená, London (GB); Nicholas McCarty, Pasadena, CA (US)

(21) Appl. No.: 17/285,989

(22) PCT Filed: Oct. 18, 2019
(86) PCT No.: PCT/GB2019/052990

§ 371 Date: Apr. 16, 2021

(30) Foreign Application Priority Data
Oct. 18, 2018 (GB) 1817010.0

(51) Int. Cl. C12N 15/11; C12N 9/22; C12N 15/90; C12N 15/66; A61K 48/00; A61K 38/46; A61K 31/7088

(52) U.S. Cl. CPC C12N 15/111; C12N 9/22; C12N 15/11; C12N 15/907; C12N 15/66; C12N 2310/20; A61K 38/465; A61K 31/7088; C12N 2330/51; C12N 2800/80; A61K 48/0066

(57) ABSTRACT

The invention provides methods for the assembly of repeated sequences that are useful in constructing nucleic acids for the simultaneous regulation and editing of multiple genes, and for DNA/RNA origami.

### FIELD OF THE INVENTION

The present invention relates to the field of RNA mediated gene regulation and gene editing, and in particular to CRISPR related methods of gene regulation. The invention also relates to methods of assembling nucleic acid polymers with repetitive domains.

### BACKGROUND

Modern DNA synthesis methods are unable to construct highly repetitive sequences, which limits the design-build-test cycle in synthetic biology.

For example, modern biotechnology and medicine requires, or at least desires, the ability to simultaneously modify the expression of multiple genes. This may be for, for example, to improve a commercial biotechnological process or to treat a disease the requires modification of the expression of multiple genes. One way of achieving this is through the simultaneous expression of multiple RNA nucleic acids to allow concerted gene repression through CRISPR interference (CRISPRi) or siRNA for example, gene activation through CRISPR activation (CRISPRa) and gene editing (CRISPR). Similarly, the field of DNA and RNA origami requires the use of multiple RNA polymers. There is also a need for simple methods of producing nucleic acid constructs that encode polypeptides that comprise repetitive sequence motifs or domains.

Current methods of achieving the co-expression of multiple RNA polymers typically require the use of a large number of vectors/plasmids, into each of which are cloned unique sequences to individually encode and express the required RNA. These multiple individual vectors/plasmids each require transformation into a target cell. However, exogenous DNA, such as plasmid/vector DNA is associated with toxicity and there is a limit to how many vectors/plasmids that a cell can harbour. In addition, the known methods are time consuming, expensive and unpredictable. The known methods are also largely species specific and modifying the constructs required for, for example, successful gene regulation in one species so that they will be compatible with another species requires multiple time consuming cloning steps.

Particularly with the advent of CRISPR, the current methods to construct arrays of gRNAs quickly, reliably and inexpensively in diverse organisms are limiting.

CRISPR has emerged as a useful tool, enabling the straightforward modification of DNA and RNA in vivo. CRISPR-Cas9, for example, performs a double-strand break (DSB) of DNA at a defined region of the genome and is directed by a short RNA sequence, called an (s)gRNA, which is a fusion of the native crRNA and tracrRNA strands². Much like TAL-effectors a decade ago, methods to construct arrays of gRNAs quickly, reliably and inexpensively in diverse organisms are limiting.

gRNAs for Cas9 are approximately 100 nucleotides in length and consist of a 20 nucleotide targeting sequence and a longer gRNA ‘scaffold’ sequence, which directs the gRNA to its corresponding endonuclease. By mutating two amino acid residues in Cas proteins, such as Cas9, CRISPR systems can instead function as transcription regulators.³ Instead of initiating a DSB, the modified Cas proteins (termed dCas9) are guided to a position in the genome, binding to the target DNA and repressing or activating transcription. Fusion to an activation or repressor domain, such as VP64 or Mxi1, respectively, enables highly effective transcriptional activation or repression of the target gene.⁴

Modulation of transcriptional targets with CRISPR-Cas approaches are currently limited by an inability to efficiently produce many different gRNAs at once in vivo, or, to efficiently product many copies of the same gRNA at once in vivo. gRNAs can be multiplexed from a single RNA transcript by encoding them in introns, flanking gRNAs with tRNAs that are cleaved by host machinery (but demand the use of Pol III promoters), or via excision of gRNAs by endoribonucleases.⁵ By flanking each gRNA with a 20 nucleotide long Csy4 recognition site and co-expressing Csy4, an endoribonuclease that recognizes this 20 nucleotide sequence and cleaves it, up to 10 gRNAs were encoded in a transcript produced from a Po III, U6 promoter in mammalian cells.⁶⁷ However, not all of these gRNAs were expressed and certainly not all of them were active.

Furthermore, there have been no reported experiments in which more than 4 gRNAs have been produced from a single promoter in the industrially-relevant model organism Saccharomyces cervisiae.⁶ Improved tools for multiplexing gRNAs in S. cerevisiae would facilitate metabolic perturbation and metabolic engineering research and expedite the ‘test’ portion of the design-build-test cycle in synthetic biology.⁸ Current challenges to multiplex gRNAs in yeast include limitations in the DNA synthesis of repetitive sequences and a shortage of auxotrophic selection markers in popular S. cerevisiae strains (such as BY4741), which demands that many gRNAs must be expressed from each locus for multiplexing experiments.⁹

The present method addresses the disadvantages of the known methods discussed above and provides a simple, quick, low-cost method of creating arrays of RNA encoding nucleic acids, all of which can be expressed from one vector/plasmid, vastly reducing the amount of nucleic acid that has to be introduced to a target cell.

The present methods can also be used to generate nucleic acids that are useful in DNA or RNA origami, and in the production of proteins or polypeptides that comprise tandem repeat sequences, repeat motifs or repeated domains, particularly where the repetitive sequences vary somewhat.

### SUMMARY OF THE INVENTION

To overcome these challenges, the inventors have invented a particular method for the construction of nucleic acid polymers that comprise repetitive domains which in particular can be used to construct nucleic acids that can be used to simultaneously generate multiple individual RNA polymers (for example multiple gRNAs) that are each separately capable of directing RNA mediated gene regulation (for example through CRISPRi or CRISPRa) or gene editing (for example by using Cas9 or a Cas9-like protein, or a Cas9/Cas9-like protein fused to a chromatin remodelling domain, or basepair exchange), for example expressing multiple gRNAs, siRNAs, or a mixture of different types of RNA polymer that directs RNA mediated gene regulation. The RNA polymers may also be useful in DNA or RNA origami. The multiple RNA polymers (for example multiple gRNAs) are expressed as a single transcript which is then cleaved into the individual RNA polymers (for example multiple gRNAs) which are then available to mediate gene regulation (for example through CRISPRi and CRISPRa). Although expressing a single RNA polymer that comprises a number of individual RNA polymers that can mediate gene regulation has previously been performed, the present invention provides new and improved methods of constructing the polymer and which can actually result in an improved polymer. For example most or all of the individual RNA polymers (for example multiple gRNAs) produced by the present method are able to mediate gene regulation. This is in contrast to prior art methods which do not allow all of the individual RNA polymers (for example multiple gRNAs) to be active, i.e. to mediate gene regulation.

### DETAILED DESCRIPTION OF THE INVENTION

The invention is defined by the claims.

The invention provides a method for producing an RNA mediated gene regulating or editing nucleic acid construct that comprises at least two sequences that are transcribed into nucleic acid polymers that each separately direct RNA mediated gene regulation or editing

wherein the at least two nucleic acid sequences are transcribed into a single transcript from a single promoter, wherein the method comprises:

a) amplifying a cassette from a gene regulating RNA generating (GRRG) vector using at least two GRRG primer pairs, each GRRG primer pair comprising a forward and a reverse primer,
wherein the GRRG vector comprises a selectable marker nucleic acid sequence and a nucleic acid sequence that when in RNA form comprises a cleavage site, optionally wherein the cleavage site is selected from:
i) an endoribonuclease cleavage site, for example a site-specific RNA endonuclease site, for example an artificial site-specific RNA endonucleases or a Csy4 cleavage sequence
ii) a tRNA sequence
iii) a ribozyme sequence
iv) an intron
v) a target sequence for an RNA directed cleavage complex
wherein the forward and reverse GRRG primers comprise nucleic acid sequences that are complementary to sequences of the GRRG and allow hybridisation of the primers to the GRRG vector at either side of the selectable marker sequence such that upon hybridisation the primers are directed away from the selectable marker nucleic acid sequence,
wherein the reverse GRRG primer hybridises to a common portion of the sequence that when in RNA form comprises a cleavage site, optionally wherein the sequence of the reverse primer is the same for each reverse primer in each primer pair, and wherein the forward GRRG primer hybridises to a common forward primer hybridisation sequence of the GRRG vector,
wherein the forward GRRG primer of each primer pair further comprises a sequence that encodes an RNA polymer that directs RNA mediated gene regulation or editing,
   which is not complementary to the vector nucleic acid sequence and which is located 5′ of the forward primer sequence that is complementary to the GRRG
wherein amplification using each of the forward and reverse GRRG primer pairs results in the production of a linear cassette that comprises the following components in the following order 5′ to 3′:
i) the sequence that encodes an RNA polymer that directs RNA mediated gene regulation or editing
ii) the forward primer hybridisation sequence
iii) the nucleic acid sequence that when in RNA form comprises a cleavage site
but which does not comprise the marker nucleic acid sequence,
optionally wherein the linear cassette comprises intervening nucleic acid located between (ii) the forward primer hybridisation sequence and (iii) the nucleic acid sequence that when in RNA form comprises a cleavage site; and

b) separately circularising each of the linear cassettes produced in step (a) to produce a circular nucleic acid polymer such that the sequence that encodes an RNA polymer that directs RNA mediated gene regulation or editing, is located between the forward primer hybridisation sequence and the nucleic acid sequence that when in RNA form comprises a cleavage site, optionally wherein the circularising comprises ligation of the two ends the linear cassette; and

c) providing at least two linking primer pairs, each primer pair comprising
a forward linking primer and a reverse linking primer,
wherein the forward linking primer is capable of hybridising to the nucleic acid sequence that when in RNA form comprises a cleavage site and the reverse linking primer is capable of hybridising to the common forward primer hybridisation sequence of the GRRG vector,
wherein each of the forward and reverse linking primers comprises a nucleic acid sequence capable of forming a single-stranded overhang, optionally wherein each primer comprises a Type II S restriction site or homing endonuclease site, wherein each pair of forward and reverse linking primers are designed so that following amplification the single-stranded overhang generated at one end of the amplification product generated by a first linking primer pair is able to hybridise with a compatible single-stranded overhang generated at one end of a second amplification product generated by a second linking primer pair;

d) amplifying each of the cassettes formed in step (b) with the appropriate pair of linking primers of (c); and

e) treating the amplification products of (d) to generate a single-stranded overhang, optionally digesting the amplification products with an appropriate Type II S restriction enzyme(s) or homing endonuclease(s); and

f) assembling the treated amplification products of (e) to one another to generate a single nucleic acid assembly comprising the assembled amplification products; and either

g) ligating the single nucleic acid of (f) to a nucleic acid destination or expression vector, optionally wherein the vector comprises a promoter sequence and optionally a terminator sequence,
optionally wherein the promoter nucleic acid sequence and/or optional terminator sequence has compatible overhangs to the ends of the single nucleic acid of (f), such that the promoter is located 5′ to the ligated amplification products of (0 and is capable of driving expression of a single transcript from the ligated amplification products and the optional terminator is located 3′ to the ligated amplification products of (f)
optionally where steps (f) and (g) are performed simultaneously; or

(h) (i) ligating the single nucleic acid of (0 to an intermediate nucleic acid vector producing an intermediate vector comprising the single nucleic acid assembly of step (f), optionally where steps (f) and (h)(i) are performed simultaneously;
(ii) performing steps (a) to (f) and (h)(i) at least twice resulting in at least two different intermediate vectors each comprising a different single nucleic acid assembly of step (f);
(iii) digesting the respective at least two intermediate vectors to produce at least two cleavage fragments comprising different nucleic acid assemblies; and/or amplifying the at least two different nucleic acid assemblies from the at least two intermediate vectors;
(iv) ligating the at least two cleavage fragments or the at least two amplification products into a single destination or expression vector producing an array of nucleic acid assemblies of (f),

wherein the destination or expression vector comprises a promoter and optionally a terminator, wherein the promoter is located 5′ to the array of nucleic acid assemblies of (f) and is capable of driving expression of a single transcript from the array, and the optional terminator is located 3′ to the array of nucleic acid assemblies of (f).

In some embodiments, the nucleic acid vector of step (g) is the destination or expression vector and comprises a promoter and a terminator suitable for driving transcription of the single nucleic acid of step (f) (i.e. the single nucleic acid which itself comprises at least two sequences that encodes a RNA polymer that directs RNA mediated gene regulation or editing). The terms destination and expression vector can be used interchangeably, and is intended to mean any vector which is suitable for the expression of the single transcript from the array, or assembly of arrays. The skilled person will understand what are the necessary properties of such a vector, for example a promoter suitable for use in a given host of cell type.

In other embodiments, the nucleic acid vector of step (h) is classed as an intermediate vector, and does not necessarily have to comprise a promoter and a terminator suitable for driving transcription of the single nucleic acid of step (f) (i.e. the single nucleic acid which itself comprises at least two sequences that encodes a RNA polymer that directs RNA mediated gene regulation or editing). In this embodiment, the “intermediate” vector serves as a framework in which to assemble multiple sequences that encode a RNA polymer that directs RNA mediated gene regulation or editing. See for example FIG. 8. Once the sequences that encodes a RNA polymer that directs RNA mediated gene regulation or editing are assembled in the intermediate vector, the whole array of sequences that encodes a RNA polymer that directs RNA mediated gene regulation or editing can be cloned out using, for example, standard restriction digestion cloning techniques, or could be amplified from the intermediate vector using, for example, PCR. It will be apparent that in some embodiments, the intermediate vector comprises appropriately placed cleavage sites, such as homing endonuclease sites or restriction enzymes sites, such as Type II restriction enzymes sites, such as BsmBI sites, so that once the array is assembled, the array can be cleaved from the vector using the appropriately placed sites, i.e. sites placed at either end of the array.

Any vector can be used as the backbone vectors of the present invention, for example the intermediate or destination/expression vectors. Examples of vectors are given in Example 4, which also highlights the different components of the vectors. The intermediate vector can be any vector, as will be apparent to the skilled person. Examples of sequences of appropriate vectors for use in the present invention are shown in SEQ ID NO: 76-84.

This embodiment is particularly advantageous when a larger array of sequences that encodes a RNA polymer that directs RNA mediated gene regulation or editing is required. For example, a first set of sequences that encodes a RNA polymer that directs RNA mediated gene regulation or editing can be assembled and cloned into a first intermediate vector. A second set of sequences that encodes a RNA polymer that directs RNA mediated gene regulation or editing (some of which may be the same as those in the first set, or alternatively all sequences may be different) can be assembled into a second intermediate vector, and so on. Any number of assemblies of sequences that encodes a RNA polymer that directs RNA mediated gene regulation or editing can be constructed in intermediate vectors. Once the arrays have been assembled into an intermediate vector, the assembly can be cut out using an appropriately placed cleavage site(s), for example as described above, for example a restriction enzyme site for example a BsmBI site, or can be amplified out of the vector using PCR. These sites are otherwise called “exit” sites, since they allow the easy exit of the nucleic acid array from the vector. The multiple arrays can then be cloned into a final destination vector, which does have the appropriate features such as promoter and terminator to drive expression across to entire assembly of multiple arrays.

It should be clear that the at least two nucleic acids of step (f) could be generated from the same, or from different, GRRG vectors.

It will be apparent to the skilled person that in assembling a final array of multiple smaller arrays (which each comprise a number of sequences that encodes a RNA polymer that directs RNA mediated gene regulation or editing) it is, in some instances, useful to ensure that a particular arrangement and direction of arrays are produced in the final vector. This is considered important to at least ensure that the direction of the array is appropriate with respect to the promoter sequence and other arrays in the assembly. The skilled person will understand that this can be achieved by using a particular sequence of cleavage sites, such as Type II restriction sites, at either side of the assembled arrays in the intermediate vector. For example, if the assembled array of a first intermediate vector is flanked by cleavage site A and B (each of which produce compatible overhangs following digestion, i.e. A-A; B-B), the assembled array of a second intermediate vector is flanked by cleavage sites B and C; the assembled array of a third intermediate vector is flanked by cleavage sites C and D; and the assembled array of a fourth intermediate vector is flanked by cleavage sites D and E, it will be readily apparent to the skilled person that digestion with enzymes A, B, C, D and E followed by ligation ought to result in an assembled array of sequences that encode a RNA polymer that directs RNA mediated gene regulation or editing which has a defined order (i.e. first array followed by second array followed by third array followed by fourth array), and wherein each array has a particular orientation 5′ to 3′. If the destination or expression vector has a cleavage site A and a cleavage site E, the assembled array of arrays can be cloned simply and directionally into the final destination vector, ready for expression.

Accordingly, in some embodiments, instead of step (g) above, the method comprises step (h)(i) as follows:

(h)(i) ligating the single nucleic acid of (f) to an intermediate nucleic acid vector producing an intermediate vector comprising the single nucleic acid assembly of step (f), optionally where steps (f) and (h)(i) are performed simultaneously;
(ii) performing steps (a) to (f) and (h)(i) at least twice resulting in at least two different intermediate vectors each comprising a different single nucleic acid assembly of step (f);
(iii) digesting the respective at least two intermediate vectors to produce at least two cleavage fragments comprising different nucleic acid assemblies; and/or amplifying the at least two different nucleic acid assemblies from the at least two intermediate vectors;
(iv) ligating the at least two cleavage fragments or the at least two amplification products into a single destination or expression vector producing an array of nucleic acid assemblies of (f),
wherein the destination or expression vector comprises a promoter and optionally a terminator, wherein the promoter is located 5′ to the array of nucleic acid assemblies of (f) and is capable of driving expression of a single transcript from the array, and the optional terminator is located 3′ to the array of nucleic acid assemblies of (f).

Where a smaller number sequences that encodes a RNA polymer that directs RNA mediated gene regulation or editing are required, the use of an intermediate vector is not required, and instead the array of sequences that encodes a RNA polymer that directs RNA mediated gene regulation or editing can be assembled straight into the final destination vector (i.e. step (g) rather than step (h)(i)-(v)).

A schematic of one exemplary way of performing the above method is indicated in FIG. 1. This figure indicates the method including step (g). FIG. 8 demonstrates the method including step (h)(i)-(iv). This Figure shows exemplary embodiments of some features in square brackets, for example the forward portion of the GRRG vector does not have to encode a Cas9 scaffold sequence.

A preferred name that can be given to the method of the invention is CHORDS (Construction of Highly Ordered and Repetitive DNA Sequences).

The method of the invention essentially involves a) the production of a number of amplification products, each of which is produced from a common template, and each of which comprises a nucleic acid sequence that when transcribed into RNA results in RNA polymers that can direct RNA mediated gene regulation or gene editing (in some other embodiments when transcribed into RNA the RNA is useful in DNA or RNA origami, or when transcribed into RNA the RNA is translated into a polypeptide), b) circularisation of the amplification products such that the unique (to each amplification product) nucleic acid sequence that when transcribed into RNA can direct RNA mediated gene regulation is flanked on either side by common nucleic acid sequence, c) and d) amplification using a common set of primers of a cassette that comprises the nucleic acid sequence that when transcribed into RNA can direct RNA mediated gene regulation or gene editing for example, e), f), and g) the sequential ordered combination of the amplification products into a single nucleic acid, followed by the incorporation of the single nucleic acid into a) a nucleic acid that is in some embodiments a final destination or expression vector that comprises a suitable promoter that can drive expression of a single transcript that comprises each of the nucleic acid sequences that when transcribed into RNA can direct RNA mediated gene regulation or editing for example; or b) in other embodiments as described above, the single nucleic acid is incorporated into an intermediate vector and optionally then subsequently a final destination vector. In a preferred embodiment this is an intelligently designed destination vector as described below. When in use, the single RNA is cleaved into individual RNA polymers by cleavage of the cleavage sites that are encoded by the GRRG and each RNA polymer is then able to direct gene regulation or gene editing.

The RNA mediated gene regulating or editing nucleic acid construct may itself comprise RNA or DNA. Typically the RNA mediated gene regulating or editing nucleic acid construct will comprise DNA.

The skilled person will understand that typically it is not the nucleic acid polymer (or portions thereof) of the RNA mediated gene regulating or editing nucleic acid construct that performs the RNA mediated gene regulation or editing. Rather, the RNA mediated gene regulating or editing nucleic acid construct comprises sequences that, once transcribed into RNA are then capable of performing the gene regulation or editing. Accordingly, in one embodiment, the RNA mediated gene regulating or editing nucleic acid construct comprises DNA that is transcribed into RNA that mediates gene regulation or editing, or in one embodiment, the RNA mediated gene regulating nucleic acid construct comprises DNA that encodes RNA that mediates gene regulation or editing.

The nucleic acid sequences that are transcribed into nucleic acid polymers that each separately direct RNA mediated gene regulation or editing are suitable for use in any method of RNA mediated gene regulation or editing. For example, in one embodiment the nucleic acid polymers that each separately direct RNA mediated gene regulation or editing are suitable for use in any one or more of CRISPR, sense Suppression/Cosuppression, antisense suppression, double-stranded RNA interference, hairpin RNA interference, intron-containing hairpin RNA interference, siRNA, micro RNA, piRNA and snoRNA methods. For example, in one embodiment the nucleic acid polymers that each separately direct RNA mediated gene regulation or editing are gRNA polymers. In another embodiment the nucleic acid polymers that each separately direct RNA mediated gene regulation or editing are siRNA polymers.

Methods of gene regulation or editing such as CRISPR, sense Suppression/Cosuppression, antisense suppression, double-stranded RNA interference, hairpin RNA interference, intron-containing hairpin RNA interference, siRNA, micro RNA, piRNA and snoRNA are well known to the skilled person and the preferences for the components and nucleic acids required to carry out the gene regulation or editing are well known. For example, microRNAs are typically about 20-23 nt in length and are found in plants, animals and certain viruses. miRNAs bind to target RNA molecules and regulate their translation but also appear to have other functions, including cleavage of target mRNAs and destabilization of target mRNAs. microRNAs are typically encoded as a miRNA stem-loop, or pre-processed miRNA. After processing by endogenous cellular machinery, a mature microRNA is released.

### EXAMPLE

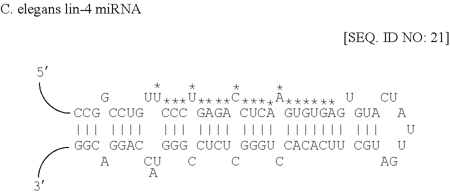

The mature miRNA is shown with (*). Using the present methods, the entire, pre-processed sequence can be added to an RNA mediated gene regulating nucleic acid construct using a single primer. (Agranat-Tamir et al 2014 NAR 42: 4640-4651).

Key proteins of the microprocessor are DGCR8, which binds the RNA molecule, and Drosha, an RNase III type enzyme, which cleaves the primary (pri) miRNA transcript into a precursor (pre) miRNA stem-loop molecule of ˜70-80 bases. In the second step, which occurs after its export by exportin-5 to the cytoplasm, the pre-miRNA is cleaved by the RNase III Dicer yielding mature miRNA and its complementary miRNA*. The miRNA is then loaded on the RNA-induced silencing complex (RISC), which directs its binding to its target gene.

Small nucleolar RNAs, or snoRNAs, are typically encoded in the introns of genes. Around 300 have been identified in the human genome. There are three types of snoRNA, the C/D box type, the H/ACA box type, and the composite H/ACA and C/D box type. The different types differ based on secondary structure of the snoRNA.

Example sequence (Homo sapiens, C/D box snoRD15A) ˜150 bp in length [SEQ ID NO: 22]

CTTCAGTGATGACACGATGACGAGTCAGAAAGGTCACGTCCTGCTCTTGGT
CCTTGTCAGTGCCATGTTCTGTGGTGCTGTGCACGAGTTCCTTTGGCAGAA
GTGTCCTATTTATTGATCGATTTAGAGGCATTTGTCTGAGAAGG

Small interfering RNA (siRNA), sometimes known as short interfering RNA or silencing RNA, is a class of double-stranded RNA molecules which are typically 20-25 base pairs in length, similar to miRNA, and operate within the RNA interference (RNAi) pathway. It interferes with the expression of specific genes with complementary nucleotide sequences by degrading mRNA after transcription, preventing translation. The sequence of the siRNA is therefore designed to be complementary to a target RNA molecule, thus impairing translation of said target RNA molecule. Sequences vary greatly, depending on target gene, but siRNAs are typically comprised of a stem-loop structure comprising a 19 bp stem and 9 nt loop with 2-3 U's at the 3 end. Design guides are readily available to the skilled person, for example at the ThermoFisher website: See: https://www.thermofisher.com/us/en/home/references/ambion-tech-support/mai-sima/general-articles/-sima-design-guidelines.html.

It will be appreciated that the RNA mediated gene regulating or editing nucleic acid construct may comprise nucleic acid sequences that are transcribed into nucleic acid polymers that each separately direct RNA mediated gene regulation or editing that are for use in the same method of RNA mediated gene regulation or editing, for example where all of the nucleic acid sequences that are transcribed into nucleic acid polymers that each separately direct RNA mediated gene regulation or editing are gRNA polymers, for example for use in CRISPRi or CRISPRa. Alternatively, the RNA mediated gene regulating nucleic acid construct may comprise nucleic acid sequences that are transcribed into nucleic acid polymers that each separately direct RNA mediated gene regulation or editing which are suitable for use in different methods of RNA mediated gene regulation or editing. For example, the polymers that each separately direct RNA mediated gene regulation or editing may comprise gRNA sequences and siRNA sequences, for example.

In one exemplary embodiment, expressing two gRNAs and a microRNA simultaneously from a single transcript and processing this transcript with DROSHA/microRNA machinery can be used to strongly inhibit Hepatitis B virus replication in vivo (see Wang et al 2017 Theranostics 7: 3090-3105). The skilled person will appreciate that this and other combinations of gene regulating or editing sequences can be incorporated into a single transcript using the methods and components of the present invention.

In one embodiment, the RNA mediated gene regulating or editing nucleic acid construct is a linear construct. It is known that linear strands of DNA transformed into cells, such as E. coli, are transcribed to RNA and can be processed into active gRNA molecules. This is advantageous in some situations, for example in situations where it is desirable to dispose of the gRNA fragments/have the cell break down the gRNAs quickly. Cells naturally dispose of linear DNA fragments if they do not possess homology arms to the genome, and so this is one method by which the skilled person can temporally control CRISPR or other RNA mediated gene regulation or editing applications.

In another preferred embodiment, the RNA mediated gene regulating or editing nucleic acid construct is a circular construct, i.e. is a circular vector/a plasmid.

The GRRG forward primer typically comprises an upstream 5′ portion that comprises the sequence that encodes an RNA mediated gene regulation or editing directing sequence and which is typically not complementary, or is typically not capable of hybridising to the GRRG, followed by a downstream 3′ portion that is complementary to a sequence of the GRRG and that allows hybridisation of the primer to the GRRG vector. The upstream 5′ portion of the forward primer may be of any length. For example may be between 5 nucleotides and 500 nucleotides in length, for example between 10 and 450, 15 and 400, and 350, 25 and 300, 30 and 280, 40 and 260, 50 and 240, 60 and 220, 70 and 200, 80 and 180, 90 and 160, 100 and 140, for example 120 nucleotides in in length. The skilled person will be able to determine the required length of the upstream 5′ portion that comprises the sequence that encodes an RNA mediated gene regulation or editing directing sequence since this will be dependent on the intended application. This upstream 5′ portion that comprises the sequence that encodes an RNA mediated gene regulation directing or editing sequence may also comprise additional sequences, such as cleavage sites.

The upstream 5′ portion of the GRRG forward primer may be referred to as a primer tail, or a 5′ tail.

By “directs RNA mediated gene regulation or editing” we include the meaning of targeting to a particular target gene or locus. For example, the RNA mediated mechanisms discussed herein are targeted to specific nucleic acids by virtue of the RNA sequence of the RNA that mediates the regulation or editing. Accordingly, the sequence of the RNA is important in defining where the regulation or editing will occur.

The upstream 5′ portion of the forward primer comprises the sequence that targets, or directs, the RNA transcript to the target gene or locus, for example this portion comprises sequence that is complementary to the intended target sequence.

In some embodiments, the sequence of the upstream 5′ portion of the GRRG forward primer is different for each forward primer of each primer pair.

In one embodiment, the sequence of the portion of the GRRG forward primer that is complementary to a sequence of the GRRG and that allows hybridisation of the primer to the GRRG vector in step (a) is the same for each forward primer of each primer pair. Alternatively, the sequence of the portion of the GRRG forward primer that is complementary to a sequence of the GRRG and that allows hybridisation of the primer to the GRRG vector in step (a) may be different for each, or for some of the, forward primers of each primer pair. Since the GRRG forward primer is the primer that comprises the sequence that encodes an RNA mediated gene regulation or editing directing sequence, a separate forward primer is required for each RNA mediated gene regulation directing or editing sequence that is required, i.e. the forward primer is typically not a common primer. Accordingly, whether the forward primer hybridises with the same portion of the GRRG or not is largely irrelevant, though, for ease and simplicity, typically the portion of the forward primer that hybridises to the GRRG vector will be the same across all of the GRRG forward primers that are used.

In some embodiments, particularly those that are for use in CRISPR methods, such as CRISPRi and CRISPRa and wherein the sequence that encodes an RNA mediated gene regulation or editing directing polymer encodes a gRNA sequence, the GRRG vector comprises a scaffold sequence that allows the gRNA to associate with a relevant polypeptide, such as a Cas9 polypeptide or Cas9-like polypeptide. In some embodiments, the sequence of the portion of the GRRG forward primer that is complementary to a sequence of the GRRG and that allows hybridisation of the primer to the GRRG comprises sequence that is complementary to at least a portion of, or all of, the scaffold sequence. Preferences for the scaffold sequence are discussed herein.

The GRRG reverse primer typically comprises a single portion that is capable of hybridising to the GRRG vector and does not comprise a portion that cannot hybridise to the GRRG vector, though in some embodiments the reverse primer may comprise additional sequence at the 5′ end, i.e. the reverse primer may comprise a 5′ tail portion.

In the same or alternative embodiment, the sequence of the GRRG reverse primer that is complementary to a sequence of the GRRG and that allows hybridisation of the primer to the GRRG vector in step (a) is the same for each reverse primer of each primer pair. As for the forward primer, the reverse primer in each pair may hybridise to the GRRG at different positions and so the reverse primer may comprise different nucleic acid sequences for each, or some of, the primer pairs. However, a strength of the present invention is that it allows the use of a common reverse GRRG primer. Accordingly, in this situation, the reverse primer can be ordered off-the-shelf, or in bulk, with no or little concern for primer design. Accordingly, in a preferred and advantageous embodiment, the sequence of the GRRG reverse primer that is complementary to a sequence of the GRRG and that allows hybridisation of the primer to the GRRG vector in step (a) is the same for each reverse primer of each primer pair.

The GRRG vector comprises a nucleic acid sequence that when in RNA form comprises a cleavage site, optionally wherein the cleavage site is selected from:
i) an endoribonuclease cleavage site, for example a site-specific RNA endonuclease site, for example an artificial site-specific RNA endonucleases or a Csy4 cleavage sequence
ii) a tRNA sequence
iii) a ribozyme sequence
iv) an intron
v) a target sequence for an RNA directed cleavage complex.

Preferably the GRRG vector comprises a Csy4 cleavage site.

The sequence of the GRRG reverse primer that is complementary to a sequence of the GRRG and that allows hybridisation of the primer to the GRRG vector is complementary to, and allows hybridisation to, at least part of, or all of, nucleic acid sequence that when in RNA form comprises a cleavage site, optionally the Csy4 cleavage sequence, the tRNA sequence, the ribozyme sequence, the intron or the target sequence for an RNA directed cleavage complex.

In a preferred embodiment the sequence of the GRRG reverse primer that is complementary to a sequence of the GRRG and that allows hybridisation of the primer to the GRRG vector allows hybridisation to the Csy4 cleavage site of the GRRG vector.

The GRRG forward and reverse primers are used in the amplification process of step (a). Since the amplification products that results from the amplification using the GRRG forward and reverse primers requires subsequent circularisation (step (b)), typically the forward and/or reverse primers comprise 5′ phosphate groups to aid in ligation.

The skilled person will understand what is meant by amplification. Typically this will involve the use of the polymerase chain reaction (PCR), though other amplification processes are known and are considered suitable for use in the present methods.

The skilled person will understand whether or not a particular sequence is capable of hybridising to another sequence or not. Typically by “capable of hybridising” we include the meaning of capable of hybridising under typical PCR conditions. For example, the relevant sequences may be capable of hybridising to one another at a temperature of between, for example 30C and 75° C., for example between 35° C. and 70° C., 40° C. and 65° C., 45° C. and 60° C., 50° C. and 55° C., for example between 55° C. and 75° C., for example around 60° C.

The amplification product of (a) can be any size. For example the amplification product of (a) can be between 200 bp and 20 kb in length, for example between 500 bp and 15 kb, 1 kb and 15 kb, 2 kb and 10 kb, 4 kb and 8 kb, for example 5 kb in length. 20 kb is considered to be the current ‘outer’ limits for fragment sizes which can be reliably amplified mutation-free via PCR with high-fidelity polymerases, such as PrimeStar, Q5 or Phusion polymerases, though this current limitation does not preclude longer fragments from being encompassed by the invention as and when improved amplification techniques are developed. The gRNA scaffold sequence for the association of a gRNA with the Cas9 protein is approximately 80 nucleotides in length. More information on the amplified domains which, once assembled into the nucleic acid construct represent repeated domains, can be found in the supplementary material of the manuscript.

Following circularisation of the amplification products of (a), a cassette is formed in which the sequence that encodes an RNA mediated gene regulation or editing directing sequence is located between the forward primer hybridisation sequence and the nucleic acid sequence that when in RNA form comprises a cleavage site.

This cassette is amplified in step (d) with the linking primers of (c). The linking primers are capable of hybridising to the cassette, and are also capable of hybridising to the GRRG since they comprise some of the same sequences. In one embodiment the forward linking primer is capable of hybridising to the nucleic acid sequence that when in RNA form comprises a cleavage site and the reverse linking primer is capable of hybridising to the common forward primer hybridisation sequence of the GRRG vector.

In one embodiment the linking primers may be considered to be Golden Gate primers, which the skilled person will understand since Golden Gate cloning is a well-known practice. Essentially, the linker primers each comprise at or towards their 5′ end a sequence that is capable of generating a single stranded overhang. For example, the primers may comprise a standard type II restriction site, for example, such as BamHI, which following digestion with the BamHI enzyme produces a single stranded overhang. However, each BamHI site is the same, and if multiple primers comprise the BamHI site then following ligation, the position of each particular amplification product within the assembly, or the orientation, will not be known. Accordingly, although essentially any restriction site may be used, preferably the site is a Type II S restriction site. Type IIS restriction enzymes comprise a specific group of enzymes which recognize asymmetric DNA sequences and cleave at a defined distance outside of their recognition sequence, usually within 1 to 20 nucleotides. This specific mode of action of Type IIS restriction enzymes is widely used for DNA manipulation techniques, such as Golden Gate cloning, enabling sequence-independent cloning of genes without the need to modify them by including compatible restriction sites (scars). Following ligation, the original recognition site is destroyed, preventing further cleavage by that enzyme. Since cleavage occurs away from the site, the sequence of the resulting overhang can be built in to each primer. In this way a series of primers can be designed so that, following amplification and digestion of the site, ligation occurs in an orderly and directional fashion, which ensures that each amplification product is correctly orientated along the length of the nucleic acid, i.e in the correct orientation for expression from the intended promoter.

In other embodiments, the sequence that is capable of generating a single stranded overhang comprises a homing endonuclease recognition sequence.

Homing endonuclease recognition sites are extremely rare. For example, an 18 base pair recognition sequence will occur only once in every 7×10¹⁰ base pairs of random sequence. This is equivalent to only one site in 20 mammalian-sized genomes.

The skilled person will understand what is meant by homing endonuclease enzymes, and some suitable examples are:

BneMS4ORFIP, F-CphI, F-EcoT3I, F-EcoT5I, F-EcoT5II, F-EcoT5IV, F-PhiU5I, F-SceI, F-SceII, F-TevI, F-TevII, F-TevIII, F-TevIV, H-DreI, H-DreI, I-AabMI, I-AchMI, 1-AniI, 1-ApeKI, I-BanI, I-BasI, I-BmoI, I-Bth0305I, I-BthII, I-BthORFAP, I-CeuI, I-ChuI, I-CmoeI, I-CpaI, I-CpaII, I-CpaMI, I-CreI, I-CreII, I-CsmI, I-CvuI, I-DdiI, I-DmoI, I-GpeMI, I-GpiI, I-GzeI, I-GzeII, I-HjeMI, I-HmuI, I-HmuII, I-LlaI, I-LtrI, I-LtrWI, I-MpeMI, I-MsoI, I-NanI, I-NfiI, I-NitI, I-NjaI, I-OmiII, I-OnuI, I-PakI, I-PanMI, I-PfoP3I, I-PnoMI, I-PogTE7I, I-PorI, I-PpoI, I-ScaI, I-SceI, I-SceII, I-SceIII, I-SceIV, I-SceV, I-SceVI, I-SceVII, I-SecIII, I-SmaMI, I-SpomI, I-SscMI, I-Ssp6803I, I-TevI, I-TevII, I-TevIII, I-TslI, I-TslWI, I-Tsp061I, I-TwoI, I-Vdi141I, -AvaI, PI-BciPI, PI-HvoWI, PI-MgaI, PI-MleSI, PI-MtuI, PI-PabI, PI-PabII, PI-PfuI, PI-PfuII, PI-PkoI, PI-PkoI, PI-PspI, PI-PspI, PI-ScaI, PI-SceI, PI-TfuI, PI-TfuII, PI-ThyI, PI-TliI, PI-TliII, PI-TmaI, PI-TmaKI, PI-ZbaI.

It is preferred if the overhang generated is a 4 nucleotide overhang, however, other lengths of overhang are also considered to be suitable for use in the invention, such as 2 nucleotide overhangs, 3 nucleotide overhangs, 5 nucleotide overhangs, 6 nucleotide overhangs, and 7 nucleotide overhangs, for example. Many Type II S restriction enzymes are known in the art. The table below provides some exemplary enzymes length of overhang generated following digestion:

TABLE 1

| | Over-hang |
|---|---|
| Enzyme | Length |
| Acul | 2 |
| Alw1 | 1 |
| Bael | 5 & 5 |
| Bbsl * | 4 |
| Bbsl-HF * | 4 |
| Bbvl | 4 |
| Bccl | 1 |
| BceAI | 2 |
| Bcgl | 2 & 2 |
| BciVI | 1 |
| BcoDI | 4 |
| BfuAI | 4 |
| Bmrl | 1 |
| Bpml | 2 |
| BpuEI | 2 |
| Bsal * | 4 |
| Bsal-HF ® v2 * | 4 |
| Bsal-HF ® * | 4 |
| BsaXI | 3 & 3 |
| BseRI | 2 |
| Bsgl | 2 |
| BsmAI | 4 |
| BsmBI * | 4 |
| BsmFI | 4 |
| Bsml | 2 |
| BspCNI | 2 |
| BspMI | 4 |
| BspQI * | 3 |
| BsrDI | 2 |
| Bsrl | 2 |
| BtgZI * | 4 |
| BtsCI | 2 |
| Btsl | 2 |
| BtslMutl | 2 |
| CspCl | 2 & 2 |
| Earl | 3 |
| Ecil | 2 |
| Esp3l * | 4 |
| Faul | 2 |
| Fokl | 4 |
| Hgal | 5 |
| Hphl | 1 |
| HpyAV | 1 |
| Mboll | 1 |
| Mlyl | 0 |
| Mmel | 2 |
| Mnll | 1 |
| NmeAlll | 2 |
| Plel | 1 |
| Sapl * | 3 |
| SfaNI | 4 |

In some embodiments, one or both of the linking primers are phosphorylated at the 5 end.

It will be appreciated that the present methods, in which the sequences that are capable of generating a single stranded overhang and which are used for the ordered ligation of the amplification products (e.g. through Golden Gate cloning) are built into primers rather than vectors, as previously used in other methods, is particularly advantageous. The present approach negates the substantial testing and optimisation required with methods that use vectors that themselves comprise the sequences that are capable of generating a single stranded overhang. The present method also negates the use of many vectors.

As discussed, the RNA mediated gene regulating or editing nucleic acid construct comprises at least two sequences that are transcribed into nucleic acid polymers that each separately direct RNA mediated gene regulation or editing. Transcription of these sequences requires a promoter. Where, for example, the RNA mediated gene regulating or editing nucleic acid construct is a linear construct, a linear promoter nucleic acid may be added to step (f) so that ligation of the promoter occurs simultaneously with ligation of the amplification products, or a linear promoter nucleic acid may be subsequently ligated to the single nucleic acid of (f).

As discussed, in some preferred embodiments, the RNA mediated gene regulating or editing nucleic acid construct is a circular construct. In this instance the promoter in step (g) may be located in a destination vector so that the ligation of step (g) results in the incorporation of the single nucleic acid of (f) that comprises the amplification products of (d) into the destination vector, under the control of the promoter. Where an intermediate vector is used (for example step (h)(i)-(iv)), the intermediate vector itself may comprise a promoter suitable for expressing the assembly of nucleic acids of (f). However, since the intermediate vector is typically itself not used for expressing the nucleic acid in the host, for example in a host cell, it is not essential that the intermediate vector comprises a promoter suitable for expressing the nucleic acid assembly.

A destination vector (otherwise called an expression vector) is essentially an end vector into which the assembled amplification products are ultimately incorporated. The destination vector can include all the necessary components for transcription, such as promoter and terminator sequences. The destination vector will also typically include a selectable marker. Examples of selectable markers are discussed herein.

Advantageously, the destination vector comprises exit cleavage sites, for example exit restriction endonuclease sites that allow the easy removal of the assembled amplification products as a single unit. The exit cleavage or restriction endonuclease sites allow straightforward transfer of the assembled fragments into other destination vectors that may comprise, for example, different promoters, terminators or other sequences. The different destination vectors may be optimised for, for example, expression and maintenance in different species, such as yeast and humans. The skilled person will be well aware of the necessary components required to produce successful expression vectors.

Preferably, in one embodiment the destination vector comprises the exit cleavage or restriction endonuclease sites. In another embodiment, the exit cleavage or restriction endonuclease sites are incorporated into the first and final linking primers of (c) such that following assembly of the amplification products, the single nucleic acid is flanked by the exit cleavage or restriction endonuclease sites.

The skilled person will appreciate that the exit site should be a low frequency site to avoid cleavage of either the destination vector backbone or the assembled amplification products.

Preferably the exit cleavage site results in the formation of single stranded overhangs. The skilled person will understand the preferences for the exit cleavage site. The cleavage site will preferably be a low frequency site, i.e. a site that does not appear often, or even at all, in the genomes of organisms, for example the target organism. In this way, the targeting RNA sequence should be able to be directed towards any target without risk of it being cleaved by the exit cleavage enzyme. For example, the exit cleavage site may be a cleavage site for a low frequency type IIs restriction enzyme or a homing endonuclease as discussed above. The skilled person has many tools available to determine the frequency of cleavage sites, for example the frequency in target genomes. Such tools are available on the New England Biolabs website, for instance. FIG. 7 shows the frequency of cleavage sites found in some commonly used DNA molecules. An exemplary exit site is an EcoRI restriction endonuclease site.

The intermediate vector used in some embodiments can share many features with the destination vector, for example can preferably comprise “exit cleavage sites”, as described herein. Properties described for the destination vector regarding the exit cleavage sites also apply to the intermediate vector.

Since for the production of RNA polymers that mediate gene regulation or editing (or in the production of nucleic acids useful in DNA or RNA origami discussed below) the transcript produced from the destination vector is not to be translated, in preferred embodiments the destination vector does not comprise a translation start codon. However, in other applications discussed below, for example in the generation of a polypeptide that comprises a tandem array of repeat motifs, the start codon is required.

The promoter that drives expression of the at least two sequences that are transcribed into nucleic acid polymers that each separately direct RNA mediated gene regulation can be any promoter. The skilled person will understand what is meant by the term promoter, and suitable promoters can be obtained from various organisms. Some promoters are species specific whilst other promoters can be used in multiple species.

Promoters are typically classed as either strong or weak depending on their affinity for RNA polymerase. The promoters used to drive expression of the at least two sequences that are transcribed into nucleic acid polymers can be a RNA Pol II promoter or a RNA Pol III promoter. Where the nucleic acid sequence that when in RNA form comprises a cleavage site is a tRNA sequence the promoter should be a RNA Pol II Promoter. However, preferably the promoter is a RNA Pol II promoter. For example, where the cleavage site is a Csy4 cleavage sequence, a ribozyme sequence or an intron, the promoter is preferably a RNA Pol II promoter.

Preferably, the promoter, whether RNA Pol II or III, is a strong promoter. By a strong promoter we include the meaning of a promoter that produces RNA molecules at a rate that is significantly faster than the average ‘promoter’ within the genome of any given organism or in vitro. The strong promoters described herein have been characterised in accordance with Lee et al 2015 ACS Synth Biol 9: 975-986 which is specifically incorporated by reference, particularly the methods relating to analysis of promoter strength under the heading “Characterization of promoters” on page 978-979. The skilled person will understand how to identify a strong promoter. For example, the strength of various promoters that are native to a particularly organism can be tested by, for example, analysing the amount of fluorescent protein produced from a gene under the control of each promoter to be tested. It will then be readily apparent to the skilled person which of these promoters are strong and which are not strong. In one embodiment a strong promoter for use in a particular organism is a promoter that produces RNA molecules at a rate that is significantly faster than the average promoter found within the genome of the particular organism. See also Qin et al 2010 PLoS One https://doi.org/10.1371/journal.pone.0010611.

Other strong promoters are considered to include the Human elongation factor 1α promoter (EF1A) and the chicken β-Actin promoter coupled with CMV early enhancer (CAGG) promoter.

In one embodiment the promoter is a RNA Pol II promoter. In a further embodiment the promoter is a strong RNA Pol I promoter. In yet a further embodiment the promoter is an inducible RNA Pol II promoter, optionally an inducible strong RNA Pol II promoter.

In one embodiment the Pol II promoter is selected from the group consisting of the TDH3 promoter, TEF1 promoter, PGK1 promoter, pCCW12 promoter, pTEF2 promoter, pHHF1 promoter, pHHF2 promoter, pALD6 promoter, Gal1 promoter, pPGK1 promoter, pHTB2 promoter or the CUP1 promoter. The Gal1 promoter is inducible by galactose and the CUP1 promoter is inducible by copper-sulphate. Tetracycline inducible promoters are also considered to be useful. In a preferred embodiment the promoter is a Pol II promoter and is a TDH3 promoter (See for example Lee et al 2015 ACS Synthetic Biology 4: 975-986).

The promoters discussed above are yeast promoters and may not work in some other organisms. However, as described in detail above, the skilled person will be able to identify suitable strong promoters for use in other organisms without undue burden. Indeed, the strength of many promoters have already been characterised as discussed above.

In one embodiment the promoter is a RNA Pol III promoter. In a further embodiment the promoter is a strong RNA Pol III promoter. In yet a further embodiment the promoter is an inducible RNA Pol III promoter, optionally an inducible strong RNA Po 111I promoter. In one embodiment the Pol III promoter is selected from the group consisting of the tRNA Phe promoter with a 5′ HDV ribozyme, the U6 promoter or the H1 promoter.

The promoter, for example the strong promoter, for use in the invention may be a naturally occurring promoter or may be a synthetic promoter.

As discussed above, the GRRG vector comprises a nucleic acid sequence that when in RNA form comprises a cleavage site, optionally wherein the cleavage site is selected from:
i) an endoribonuclease cleavage site, for example a site-specific RNA endonuclease site, for example a Csy4 cleavage sequence or an artificial site-specific RNA endonuclease cleavage sequence
ii) a tRNA sequence
iii) a ribozyme sequence
iv) an intron
v) a target sequence for an RNA directed cleavage complex.

It will be clear to the skilled person that the requirement for this sequence is simply that, once transcribed into RNA, it is capable of being specifically cleaved, for example cleaved by an enzyme. There are various ways in which this can be achieved.

For example, site-specific RNA endonucleases exist, for example artificial Site-specific RNA endonucleases, or ASREs, see for example Choudhury et al 2012 Nature Communications 3 Article 1147; and Zhang et al 2013 Molecular Therapy 22(2) 312-320. The use of such enzymes and the accompanying recognition sequences are encompassed in the present invention.

Another RNA specific endonuclease is Csy4 which is a CRISPR endonuclease that processes RNA. Specifically, Csy4, in native bacterial systems (such as Pseudomonas aeruginosa) processes pre-crRNA transcripts by cleaving a specific, 28 nucleotide long stem-and-loop sequence of RNA. Csy4 specifically cleaves only its cognate pre-crRNA substrate.

Recognition of its cognate pre-crRNA substrate is mediated, in part, by interactions with the following amino acid residues in the Csy4 protein: Q104, A19, U7, G20, C6, F155, R102. See for example Haurwitz et al Science. 2010 Sep. 10; 329(5997):1355-8. doi: 10.1126/science.1192272.

The Csy4 cleavage site for use in the invention is considered to be a 20 nucleotide cleavage site, or a 28 nucleotide cleavage site. The Csy4 protein only cleaves the site in RNA, not in DNA. Accordingly, it will be understood that where the GRRG vector is DNA, the Csy4 protein does not cleave the DNA vector, but only cleaves the RNA transcript produced from the destination vector, into which the nucleic acid that encodes the Csy4 protein in incorporated. Table 2 and SEQ ID NO: 1-4 provide sequence information for the DNA and RNA Csy4 site sequences. The skilled person will understand that some variation in these sequences may be tolerated and still allow the Csy4 protein to cleave the site.

Accordingly, in one embodiment the GRRG vector comprises a nucleic acid sequence of SEQ ID NO: 1 or SEQ ID NO:2.

In other embodiments, the cleavage site is a pre-tRNA sequence. tRNA sequences are cleaved in eukaryotes by RNase P and RNase Z (or RNase E in bacteria), which removes excess 5′ and 3′ sequences. These enzymes recognize the tRNA secondary structure, so must be expressed to cleave ANY desired tRNA sequence. See Shiraki and Kawakami 2018 Scientific Reports 8: 13366.

The following shows some exemplary tRNA sequences along with the 5′ leader sequence.

pre-tRNA^{Gly}:
[SEQ ID NO: 5]
5′-AACAAAGCACCAGTGGTCTAGTGGTAGAATAGTACCCT
Dr-RNAGly(GCC)]
[SEQ ID NO: 6
gtgaGCATTGGTGGTTCAGTGGTAGAATTCTCGCCTGCCACGCGGGAGGCC
CGGGTT CGATTCCCGGCCAATGCA
Dr-tRNALys(CTT)
[SEQ ID NO: 7]
gttctcatcaGCCCGGCTAGCTCAGTCGGTAGAGCATGAGACTCTTAATCT
CAGGGTCGTG GGTTCGAGCCCCACGTCGGGCG
Dr-tRNAAsn(GTT)
[SEQ ID NO: 8]
gctatctGTCTCTGTGGCGCAATCGGTTAGCGCGTTCGGCTGTTAACCGAA
AGGTTGGTGGTTCGAGCCCACCCAGGGACG
Dr-tRNAMet(CAT)
[SEQ ID NO: 9]
gcctgaagGTTTCCGTAGTGTAGTGGTTATCACGTTCGCCTCATACGCGAA
AGGTCCCCA GTTCGAAACTGGGCGGAAACA
Dr-tRNAGln(CTG)
[SEQ ID NO: 10]
gacttgaGGTTCCATGGTGTAATGGTTAGCACTCTGGACTCTGAATCCAGC
GATCCGAGT TCAAATCTCGGTGGGACCA
Dr-tRNASer(GCT)
[SEQ ID NO: 11]
ggaaaatGACGAGGTGGCCGAGTGGTTAAGGCGATGGACTGCTAATCCATT
GTGCTTTG CACGCATGGGTTCGAATCCCATCCTCGTCG
Dr-tRNAThr(AGT)
[SEQ ID NO: 12]
gcagcGGCGCCGTGGCTTAGTTGGTTAAAGCGCCTGTCTAGTAAACAGGAG
ATCCTGG GTTCGAATCCCAGCGGTGCCT
Dr-tRNAHis(GTG)
[SEQ ID NO: 13]
gctcGCCGTGATCGTACAGTGGTTAGTACTCTGCGTTGTGGCCGCAGCAAC
CCCGGTT CGAATCCGGGTCACGGCA
Dr-tRNALeu(CAG)
[SEQ ID NO: 14]
gcatGTCAGGATGGCCGAGTGGTCTAAGGCGCTGCGTTCAGGTCGCAGTCT
CCCCTG GAGGCGTGGGTTCGAATCCCACTTCTGACA
Os-tRNAGly(GCC)
[SEQ ID NO: 15]
gaacaaaGCACCAGTGGTCTAGTGGTAGAATAGTACCCTGCCACGGTACAG
ACCCGGG TTCGATTCCCGGCTGGTGCA
Shiraki and Kawakami 2
Os-IRNAGly(GCC)-scrambled
[SEQ ID NO: 16]
GAACCTCTTACACGCGCAGATCAACTAAATGTACACTGCGACGGTCCGTGG
CTCCGA GAGGGGTTACAGGGTACGCTG
>Dr-tRNAGly(GCC)-scrambled
[SEQ ID NO: 17]
GCGCTGTGGCGTACCGGGTACGTACTCGCTTGACTGGGTTGGTACTAGGCG
AAACC AGCTCCGTGGGATTGCACC

The nucleic acid sequence that when in RNA form comprises a cleavage site may also be a ribozyme cleavage site. The skilled person will understand preferences for ribozymes. Exemplary ribozymes and the associated sequences include:

Hammerhead ribozyme (HH)
[SEQ ID NO: 18]
gttccccCTGATGAGTCCGTGAGGACGAAACGAGTAAGCTCGTC
Hepatitis delta virus ribozyme (HDV)
[SEQ ID NO: 19]
GGCCGGCATGGTCCCAGCCTCCTCGCTGGCGCCGGCTGGGCAACATGCTTC
GGCAT GGCGAATGGGAC

As discussed above, the nucleic acid sequence that when in RNA form comprises a cleavage site may also be and intron. Intron sequences are naturally present in some genes. These native genetic promoters have been adapted for use in gRNA multiplexing (e.g. in rice plants, the UBI10p promoter is used; the 5′ UTR of this promoter has a conserved intron). The skilled person will understand what is required to put this embodiment into practice. See for example “Engineering Introns to Express RNA Guides for Cas9- and Cpf1-Mediated Multiplex Genome Editing” by Ding D. et al. 2018 Mol Plant. 11(4):542-552. doi: 10.1016/j.molp.2018.02.005. Epub 2018 Feb. 17. The intron sequence provided in Table 2 SEQ ID NO: 20 has been taken from this paper.

As discussed above, the only requirement for the sequence that when in RNA form comprises a cleavage site is that it is cleaved. It will be appreciated that the sequence of this region of the GRRG can actually be of any sequence, and this sequence can be cleaved by a RNA directed cleavage complex, as siRNA for example an siRNA complexed with Ago2. When using nucleic acid constructs which include such cleavage sites, the appropriate RNA polymers, for example siRNAs, have to be co-expressed. In some embodiments, the GRRG can be used to produce a nucleic acid construct that comprises sites for, for example RNA directed cleavage, wherein the RNA species or transcript that directs the cleavage is encoded with the same nucleic acid construct. In this way, the nucleic acid construct can essentially be self-processed using self-encoded RNA molecules in combination with co-expressed proteins, for example Ago2.

The skilled person will appreciate that the nucleic acid construct of the invention can comprise any number of sequences that are transcribed into nucleic acid polymers that each separately direct RNA mediated gene regulation or editing. For example, the nucleic acid construct of the invention may comprise between 3 and 100 nucleic acid sequences that are transcribed into nucleic acid polymers that each separately direct RNA mediated gene regulation or editing, wherein the between 3 and 100 nucleic acid sequences are expressed as a single transcript from a single promoter; optionally wherein the nucleic acid construct comprises between 5 and 95, 10 and 90, 15 and 85, 20 and 80, 25 and 75, 30 and 70, 35 and 65, 40 and 60, 45 and 55 nucleic acid sequences that are transcribed into nucleic acid polymers that each separately direct RNA mediated gene regulation or editing.

In one embodiment the nucleic acid construct of the invention comprises at least 6, 7, 8, 9, 10, 11, 12, 13, 14, 15, 16, 17, 18, 19, or at least 20 nucleic acid sequences that are transcribed into nucleic acid polymers that each separately direct RNA mediated gene regulation or editing. In one embodiment the nucleic acid construct of the invention comprises at least 11 or at least 12 nucleic acid sequences that are transcribed into nucleic acid polymers that each separately direct RNA mediated gene regulation or editing.

In some embodiments, the nucleic acid construct of the invention comprises 6 nucleic acid sequences that are transcribed into nucleic acid polymers that each separately direct RNA mediated gene regulation. It is considered that by using the method of the invention, it is relatively simply to produce a nucleic acid construct of the invention comprising up to around 6 nucleic acid sequences that are transcribed into nucleic acid polymers that each separately direct RNA mediated gene regulation, by for example following step (g) of the method. However, as described in step (h) of the invention, by employing two or more intermediate vectors, it is possible to combine arrays of nucleic acid sequences that are transcribed into nucleic acid polymers that each separately direct RNA mediated gene regulation into a longer assembly comprising more nucleic acid sequences that are transcribed into nucleic acid polymers that each separately direct RNA mediated gene regulation. For example, in one embodiment the nucleic acid construct of the invention comprises up to 6 nucleic acid sequences that are transcribed into nucleic acid polymers that each separately direct RNA mediated gene regulation, or up to 12 nucleic acid sequences that are transcribed into nucleic acid polymers that each separately direct RNA mediated gene regulation, or up to 18 nucleic acid sequences that are transcribed into nucleic acid polymers that each separately direct RNA mediated gene regulation, or up to 24 nucleic acid sequences that are transcribed into nucleic acid polymers that each separately direct RNA mediated gene regulation, or up to 30 nucleic acid sequences that are transcribed into nucleic acid polymers that each separately direct RNA mediated gene regulation, or up to 36 nucleic acid sequences that are transcribed into nucleic acid polymers that each separately direct RNA mediated gene regulation, or up to 42 nucleic acid sequences that are transcribed into nucleic acid polymers that each separately direct RNA mediated gene regulation, or up to 48 nucleic acid sequences that are transcribed into nucleic acid polymers that each separately direct RNA mediated gene regulation.

The skilled person will understand that the only limit to the number of nucleic acid sequences that are transcribed into nucleic acid polymers that each separately direct RNA mediated gene regulation or editing that can be encoded and expressed by the nucleic acid of the invention are practical limits associated with for example assembling large numbers of fragments, and the length of an RNA transcript that can be produced. Accordingly, it is feasible that the nucleic acid construct of the invention can comprise at least 200, or at least 300, 400, 500, 1000, 2000 or more sequences that are transcribed into nucleic acid polymers that each separately direct RNA mediated gene regulation or editing.

One means of producing a nucleic acid of the invention that comprises larger numbers of sequences that are transcribed into nucleic acid polymers that each separately direct RNA mediated gene regulation or editing is to use hierarchical assembly, for example to repeat method steps (a) to (f) at least once, to produce a further single nucleic acid that comprises the assembled amplification products. These at least two single nucleic acids can be ligated together by any means, and ligated to a linear promoter or incorporated into a destination vector. For example, in one embodiment method steps (a) to (f) are repeated at least once to produce a second single stranded nucleic and wherein the second single nucleic acid is ligated into the single nucleic acid that comprises a promoter of step (g).

An alternative to the above is provided in step (h), where at least two different single nucleic acids of step (t) are each individually cloned into separate intermediate vectors, and then subsequently cloned out or amplified, and combined in a single destination or expression vector.

A particular issue with producing a nucleic acid, for example a DNA nucleic acid that encodes a single transcript that itself comprises multiple individual RNA nucleic acids, is that the resultant nucleic acid often comprises repetitive sequence. Repetitive nucleic acid sequences are inherently unstable and limit the number of repeat units that can be incorporated into a single nucleic acid. It will be appreciated that the present method results in a nucleic acid of the invention that comprises repetitive sequences. For example, each of the amplification products that are assembled in step (f) comprise the sequence that encodes an RNA mediated gene regulation or editing directing sequence located between the forward primer hybridisation sequence and the nucleic acid sequence that when in RNA form comprises a cleavage site. Typically, the forward primer hybridisation sequence (which in some embodiments is a scaffold sequence as discussed herein) and the sequence that comprises a cleavage site (for example the Csy4 site) are the same between amplification products derived from different primer pairs, since typically the sequence of the GRRG forward and reverse primers that are complementary to a sequence of the GRRG and that allow hybridisation of the primers to the GRRG vector are the same across each primer pair. Each of the amplification products may also comprise the same intervening nucleic acid sequence (e.g. part of the GRRG vector backbone). Accordingly, upon assembly of the amplified products, the single nucleic acid that is generated comprises a tandem array of partially identical sequences. The method of the invention may therefore be considered to be particularly suitable for the production of constructs that comprise repetitive nucleic acid sequences.

In one embodiment of the method of the invention, the nucleic acid construct that comprises at least two sequences that are transcribed into nucleic acid polymers that each separately direct RNA mediated gene regulation or editing comprises repetitive nucleic acid sequences, for example the nucleic acid construct comprises at least two sequences that have between 75% and 100%, optionally between 80% and 99%, 82% and 98%, 84% and 97%, 86% and 96%, 88% and 95%, 90% and 94%, 91% and 93%, optionally 92% homology and/or sequence identity to one another, for example wherein the two sequences are between 5 and 100 nucleotides in length, optionally between 10 and 90, 20 and 80, 30 and 70, 40 and 60 or 50 nucleotides in length.

In one embodiment, the Csy4 recognition site is 20 nucleotides long ([SEQ ID NO: 1] provides the sequence of the DNA that encodes the Csy4 site, [SEQ ID NO: 3] provides the RNA sequence of the site), or in another or the same embodiment it is 28 nucleotides long ([SEQ ID NO: 2] provides the sequence of the DNA that encodes the Csy4 site, [SEQ ID NO: 4] provides the RNA sequence of the site). In one particular embodiment, the Cas9 scaffold domain that is in one embodiment part of the GRRG and which forms one end of the amplified products that are assembled in step (f) is 80 nucleotides in length. Accordingly, in one particular embodiment, the assembled single nucleic acid comprises a series of amplification product sequences that encodes an RNA mediated gene regulation or editing directing sequence, each flanked on one side by a 20 nucleotide or 28 nucleotide Csy4 recognition site, and on the other side by an 80 nucleotide gRNA scaffold sequence, for example a scaffold sequence for association with the Cas9 polypeptide. At the very end of each amplification product sequence is a sequence capable of forming a single-stranded overhang, for example a Type II S restriction site. For example, where the Type II S restriction site is for BsmBI, the sequence capable of forming a single-stranded overhang is 6 nucleotides in length.

In this particular embodiment, this means that a portion of nucleic acid that is 112 nucleotides or 120 nucleotides is repeated in the single nucleic acid that comprises the assembled amplification products, wherein each repeat is separated by the sequence that encodes an RNA mediated gene regulation directing sequence.

It will be appreciated that gRNAs and other RNA transcripts that direct gene regulation or editing can function as truncated or expanded RNA polymers. In one embodiment therefore the Cas9 scaffold domain that is in one embodiment part of the GRRG and which forms one end of the amplified products that are assembled in step (f) is between 20 and 150 nucleotides in length, for example between around 30 and 140, 40 and 130, 50 and 120, 60 and 110, 70 and 100, 80 and 90 nucleotides in length.

Accordingly the single nucleic acid comprises regular repeats of a sequence with the same nucleic acid sequence or of a nucleic acid sequence with between 75% and 100%, optionally between 80% and 99%, 82% and 98%, 84% and 97%, 86% and 96%, 88% and 95%, 90% and 94%, 91% and 93%, optionally 92% homology and/or sequence identity to each other, interspersed by a non-repetitive nucleic acid sequence.

In some embodiments the nucleic acid construct produced by the claimed method comprises between 3 and 100 repetitive nucleic acid sequences, for example between 5 and 95, 10 and 90, 15 and 85, 20 and 80, 25 and 75, 30 and 70, 35 and 65, 40 and 60, 45 and 55 repetitive nucleic acid sequences;
for example at least 6, 7, 8, 9, 10, 11, 12, 13, 14, 15, 16, 17, 18, 19, or at least 20 repetitive nucleic acid sequences,
for example at least 11 or at least 12 repetitive nucleic acid sequences
for example wherein the at least two sequences are between 5 and 100 nucleotides in length, optionally between 10 and 90, 20 and 80, 30 and 70, 40 and 60 or 50 nucleotides in length.

In one embodiment the length of the nucleic acid sequences that encode RNA mediated gene regulation or editing directing sequence(s) is between around 5 and 100 nucleotides in length, optionally between 10 and 90, 20 and 80, 30 and 70, 40 and 60 or 50 nucleotides in length.

In one embodiment, the length of the amplification products of steps (d) and (e) are between around 5 and 100 nucleotides in length, optionally between 10 and 90, 20 and 80, 30 and 70, 40 and 60 or 50 nucleotides in length.

It will be apparent to the skilled person that the nucleic acid sequences that encode an RNA mediated gene regulation directing or editing sequence(s) can be directed towards the exact same sequence (e.g. targeting the same sequence of the same gene), be directed towards the same gene but comprise different sequences, or can be directed towards different genes, for example for simultaneous regulation or editing of a number of genes. It will also be apparent that a single nucleic acid construct made by the method of the invention can comprise sequences that are directed towards the same gene, and also sequences that are directed towards different genes.

In one embodiment the at least two nucleic acid sequences that encode an RNA mediated gene regulation directing or editing sequence(s) are directed towards different genes, for example wherein each nucleic acid sequence that encodes an RNA mediated gene regulation or editing directing sequence is directed towards a different gene. In this embodiment some of the nucleic acid sequences that encode an RNA mediated gene regulation or editing directing sequence(s) may be directed towards the same gene, and some of the nucleic acid sequences that encode an RNA mediated gene regulation or editing directing sequence(s) may be directed towards other genes. For example, the nucleic acid produced made by the method of the invention may comprise at least 1, 2, 3, 4, 5, 6, 7, 8, 9, 10 or more nucleic acid sequences that encode an RNA mediated gene regulation or editing directing sequence(s) that are directed towards the same gene, and may comprise at least 1, 2, 3, 4, 5, 6, 7, 8, 9, 10 or more nucleic acid sequences that encode an RNA mediated gene regulation or editing directing sequence(s) that are directed towards another gene. Each of the sequences may be directed towards a different gene. In one example the nucleic acid may comprise three sequences directed towards a first gene, three sequences directed towards a second gene, three sequences directed towards a third gene, and three sequences directed towards a fourth gene, for example.

In another embodiment, the at least two nucleic acid sequences that encode RNA mediated gene regulation or editing directing sequences are directed towards the same gene, for example in one embodiment each nucleic acid sequence that encodes an RNA mediated gene regulation or editing directing sequence is directed towards the same gene.

In yet another embodiment, at least two of the nucleic acid sequences that encode an RNA mediated gene regulation or editing directing sequence are directed towards the same gene, and wherein at least one further nucleic acid sequence that encodes an RNA mediated gene regulation or editing directing sequence is directed towards a different gene.

One advantage of the present invention is that the method requires a single template nucleic acid, the GRRG vector, to generate nucleic acids with any number of, and any combination of, sequences that are transcribed into nucleic acid polymers that separately direct RNA mediated gene regulation or editing, since the unique sequences that encode the sequences that separately direct RNA mediated gene regulation or editing are contained within the GRRG forward primer. The GRRG vector itself can comprise any vector backbone. Typically the vector will be maintained in bacteria, such as E. coli and so accordingly in one embodiment the GRRG vector will be a bacterial cloning vector and will comprise all of the necessary components for maintenance and propagation in bacteria. These components will be apparent to the skilled person. One of these components is an antibiotic resistance selection marker. This resistance marker is in addition to the selectable nucleic acid described in step (a) of the method and is simply there to allow propagation of the vector in bacteria, for example. Suitable antibiotic resistance markers will be apparent to the skilled person and include, for example hygromycin resistance marker, a kanamycin resistance marker, a chloramphenicol resistance marker or an ampicillin resistance marker. Other components include a bacterial ColE1 origin of replication or other origin of replication.

It will be apparent to the skilled person that to work the invention, the actual GRRG vector per se is not required, and the amplification step (a) can be performed on an isolated fragment of the GRRG vector or a nucleic acid fragment that has a nucleic acid sequence that corresponds to the relevant part of the GRRG vector. i.e. the amplification step (a) can be performed on a linearized GRRG or equivalent nucleic acid. However, typically the amplification will be performed using a circular GRRG vector as a template simply because it is straight forward to isolate the vector from bacteria, or, the amplification can be performed on a bacterial cells that comprise the GRRG vector, for example through colony PCR.

The purpose of the selectable marker nucleic acid of the GRRG vector mentioned in step (a) is to provide an indicator of successful and appropriate amplification of the correct fragment from the GRRG and subsequent circularisation of the product. As indicated in step (a) and FIG. 1, the GRRG primers hybridise to the GRRG either side of the selectable marker, but which are orientated so that each primer is directed away from the selectable marker. This arrangement results in a linear PCR fragment that does not comprise the selectable marker. Following circularisation of the amplification product and transformation into bacteria for further cloning and maintenance, for example E. coli, the drop-out of the marker can be used to identify E. coli that comprise the correct product and not, for example, original GRRG vector that has been carried over.

It is not essential to transform the circularised amplification product into bacteria, for example E. coli, though this step is considered to increase the efficiency of the downstream steps. Accordingly, in a preferred embodiment, the method of the invention includes the step of identifying circularised products in which the marker has been dropped out, for example through the transformation of E. coli with the products of step (b) and subsequent selection of colonies in which it is evident that the marker has been lost. A further preferred step is to sequence the circularised product to verify the sequence.

The marker nucleic acid that is used to select correctly circularised products can be any marker nucleic acid. In one embodiment the marker nucleic acid encodes:
a) a positive selection marker, for example selected from the group consisting of antibiotic resistance markers optionally a hygromycin resistance marker, a kanamycin resistance marker, a chloramphenicol resistance marker or an ampicillin resistance marker: or
b) a negative selection marker, for example selected from the group consisting of rpsL, SacB and pheS; or
c) a visible selection marker, for example selected from the group consisting of LacZ or a fluorescent protein marker, for example GFP, for example superfolded GFP.

As discussed above, in one embodiment, the sequence of the GRRG to which the forward GRRG primer hybridises does not form part of the nucleic acid that directs RNA mediated gene regulation. In this embodiment, the RNA mediated gene regulating or editing nucleic acid is entirely encoded by the 5′ portion of the forward primer which is not complementary to the GRRG vector sequence. This approach is suitable for most RNA mediated gene regulation applications, such as CRISPR, sense Suppression/Cosuppression, antisense suppression, double-stranded RNA interference, hairpin RNA interference, intron-containing hairpin RNA interference, siRNA, micro RNA (miRNA) piRNA and snoRNA. This method is only limited by the length of the forward primer that can be generated. Primers of 200 nucleotides can readily be generated, meaning that RNA mediated gene regulating nucleic acids of up to 200 nucleotides or more can be incorporated into the forward primer. For example, for CRISPRi and CRISPRa, the 5′ portion of the forward primer can encompass sequences that encode both the crRNA and tracrRNA sequences of the gRNA. The tracrRNA is also known as a scaffold sequence since it allows association with Cas proteins or other associated proteins. As mentioned above, the Cas9 scaffold is around 80 nucleotides in length and the crRNA can be 20 nucleotides in length. Both of these sequences can be comfortably incorporated into the tail of a primer. Accordingly, in one embodiment the forward GRRG primer contains a nucleic acid sequence that encodes a scaffold sequence that when in RNA form allows association of the RNA with a polypeptide capable of regulating or editing a gene. In one embodiment the polypeptide is selected from the group consisting of:

Cas9 or a Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

The Cpf1 protein has a short scaffold of 20 nucleotides in length and is very AT-rich, meaning that the Tm of the primer binding is too low for appropriate use in a PCR amplification method. However, for such situations the skilled person will realise that the scaffold can be directly added in the forward primer along with the targeting sequence.

In a further embodiment, the forward GRRG primer contains the entire sequence required to encode a full gRNA sequence, optionally wherein the gRNA can associate with a polypeptide capable of regulating or editing a gene, for example in one embodiment the polypeptide is selected from the group consisting of: Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

In other embodiments, the forward GRRG primer contains an entire siRNA sequence, or an entire sense Suppression/Cosuppression, antisense suppression, double-stranded RNA interference, hairpin RNA interference, intron-containing hairpin RNA interference, siRNA or micro RNA sequence, piRNA and snoRNA.

However, in some embodiments, part of the sequence that encodes the nucleic acid that directs RNA mediated gene regulation or editing is incorporated in to the GRRG. These embodiments are considered to be useful where the sequence that encodes the nucleic acid that directs RNA mediated gene regulation or editing needs to be particularly long, for example. Other advantages of this embodiment are that the forward primer can comprise a much shorter tail and only encompass sequences that are unique to that particular sequence that encodes the nucleic acid that directs RNA mediated gene regulation or editing.

For CRISPRi, CRISPRa and CRISPR editing, the sequence that encodes the sequence that associates with a Cas9 or Cas9 like protein, i.e. the Cas9 or Cas9 like scaffold sequence, are common to all primer pairs. Accordingly, in one embodiment the GRRG vector comprises a sequence that encodes the Cas9 or Cas9 like scaffold sequence, or encodes part of the Cas9 or Cas9 like scaffold sequence. In this way, the targeting sequence, i.e. the crRNA part of the gRNA can be incorporated into the primer tail and can be much shorter, for example around 20 nucleotides, meaning that the entire forward primer may only be less than around 30 nucleotides in length, for example less than 35 nucleotides in length, for example around less than 40 nucleotides in length. In these embodiments, the forward GRRG primer hybridises to the Cas9 or Cas9 like scaffold encoding sequence of the GRRG vector, or hybridises to at least part of the Cas9 or Cas9 like scaffold encoding sequence of the GRRG vector.

Accordingly, in one embodiment, the GRRG vector comprises a scaffold sequence that when in RNA form allows association of the RNA with a polypeptide capable of regulating or editing a gene, for example in one embodiment the polypeptide is selected from the group consisting of:
Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

The skilled person will understand that between the steps (a)-(g) or (h) outlined above, other steps can be taken, such as gel purification of an amplification product or clean up with commercially available kits, which can aid in accurate cloning. For example, following step (a) and/or (b) and/or (d) and/or (e) and/or (f) the products may be gel purified or cleaned up with a kit.

The method for producing an RNA mediated gene regulating or editing nucleic acid construct of the invention is considered to be particularly advantageous over the prior art methods since the present method is considered to result in each of the constituent sequences that direct RNA mediated gene regulation or editing actually being processed into active RNA polymers and which each result in gene regulation. In the prior art methods, not all of the individual RNA polymers were found to be active.

It will be apparent that the above discussion typically relates to DNA nucleic acid which encodes sequences that, once in RNA form, are capable of mediating gene regulation.

Preferences for the features described above, including but not limited to, the type of nucleic acid (DNA or RNA; linear or circular), type of gene regulation, size and number/frequency of nucleic acid fragments, position of primer hybridisation sites, cleavage sites, lining primers, promoters and destination vectors, and other features, apply equally to all aspects and embodiments described below.

The invention also provides methods of using the nucleic acid that has been constructed using the method of the invention. For example, the nucleic acid construct can be used to express the corresponding RNA transcript, which can be processed into the individual nucleic acids that are capable of mediating gene regulation or editing.

Accordingly, the invention provides a method of producing at least two nucleic acid sequences that each separately direct RNA mediated gene regulation or editing wherein the method comprises expressing an RNA transcript from the RNA mediated gene regulating or editing nucleic acid construct produced by any of the methods described herein.

The method may produce any number of nucleic acid sequences that direct RNA mediated gene regulation or editing, as discussed above. For example, in one embodiment the method may produce between 3 and 100 nucleic acid polymers each separately direct RNA mediated gene regulation or editing, for example between 5 and 95, 10 and 90, 15 and 85, 20 and 80, 25 and 75, 30 and 70, 35 and 65, 40 and 60, 45 and 55 nucleic acid polymers that each separately direct RNA mediated gene regulation or editing.

In one embodiment the method may produce at least 6, 7, 8, 9, 10, 11, 12, 13, 14, 15, 16, 17, 18, 19, or at least 20 nucleic acid polymers that each separately direct RNA mediated gene regulation or editing. In one embodiment the method produces at least 11 or at least 12 nucleic acid polymers that each separately direct RNA mediated gene regulation or editing.

As discussed above each nucleic acid sequences that each separately direct RNA mediated gene regulation or editing is expressed from a single promoter as a single transcript. In order to liberate each of the individual RNA nucleic acid polymers so that they are able to perform the required gene regulation or editing function, the single transcript requires processing. As will be apparent from the above, between each or the nucleic acid polymer sequences that perform the gene regulation or editing are cleavage sites. Preferences for the cleavage sites are as discussed previously. Preferably the cleavage site is a Csy4 site. Accordingly, to ensure that the transcript is processed, in one embodiment the method comprises expressing the transcript in the presence of an agent that is capable of cleaving the cleavage site. For example in one embodiment the transcript may be co-expressed with the Csy4 polypeptide, or a relevant ribozyme. Cleavage of tRNA sequences is considered to occur through the innate cell components. Accordingly, where the transcript that comprises tRNA sequences is expressed in a cell, no additional components are considered to be necessary for cleavage. However, if expression of the transcript is being performed in vitro, then additional components will be required. The components required to cleave tRNA sites are well known to the skilled person, such as RNAse enzymes.

Where the cleavage site is an intron, additional agents to facilitate cleavage may be required, particularly if the transcript is expressed in bacteria which do not natively comprise introns and lack the splicing machinery of eukaryotes. The skilled person is aware of the agents necessary for splicing.

Expression of the agent that is capable of cleaving the cleavage site can be driven by any promoter, but preferably a strong promoter is used. Preferences for strong promoters are described herein. In a preferred embodiment the promoter that drives expression of the agent that is capable of cleaving the cleavage site is driven by the HHF2 promoter, for example expression or co-expression of the Csy4 polypeptide is driven by the HHF2 promoter. See Lee et al 2015 ACS Synthetic Biology 4: 975-986.

Rather than co-expressing the transcript with an agent, e.g. expressing the transcript and the agent in the same cell, the method is also considered to work if the transcript is otherwise exposed to an agent that can cleave the site, for example exposed to Csy4. Accordingly, this method is considered suitable for in vitro use, where the relevant factors are added to the transcript.

In one embodiment the method of producing at least two nucleic acid sequences that each separately direct RNA mediated gene regulation is an in vitro method.

In another embodiment the method of producing at least two nucleic acid sequences that each separately direct RNA mediated gene regulation is an in vivo method. For example, the method may be performed in a cell, a tissue, an organ or a whole organism, such as a human.

To perform the method in vivo, in one embodiment the RNA mediated gene regulating or editing nucleic acid construct must be transformed into a cell. Accordingly, in one embodiment the method further comprises transforming the RNA mediated gene regulating or editing nucleic acid construct produced by the methods described above into a cell. Also as discussed above, in some embodiments the cell expresses or comprises or is exposed to an agent that is capable of cleaving the sequence that when in RNA form is specifically cleavable, optionally in the presence of Csy4.

It will be apparent to the skilled person that the cell may be any cell. The skilled person is well equipped to design the relevant components of the method, for example the GRRG and the destination vector so as to allow expression of the transcript in any particular cell type. For example the skilled person will know to use a promoter that is active in human cells when trying to express the transcript in human cells.

In one embodiment the cell that expresses the transcript is a eukaryotic cell, for example a mammalian cell, for example a human cell, or a yeast cell, for example a S. cerevisiae cell, a Pichia pastoris cell, a Kluyveromyces lactis cell, a Yarrowia lipolytica cell or a Rhodosporidium toruloides cell. In a preferred embodiment the cell is a S. cerevisiae cell.

In other embodiments, the cell that expresses the transcript is a prokaryotic cell, for example an E. coli cell or a B. subtilis cell. Again, all that is required to allow the methods to produce a nucleic acid capable of expressing the transcript in bacteria is some minor cloning to ensure that the correct promoters and terminators are used, along with co-expression of the appropriate endoribonuclease, for example Csy4, or appropriate ribozyme, for example.

As discussed above, an advantage of the present invention is that once the single nucleic acid that comprises the at least two sequences that are transcribed into nucleic acid polymers that each separately direct RNA mediated gene regulation or editing has been assembled, it is very easy to move this nucleic acid cassette into other vectors that may comprise, for example, different promoters for expression in different species.

It will be clear to the skilled person that the expression of multiple RNA nucleic acids that can each separately mediate gene regulation has a number of uses, for example in industry or medicine.

Accordingly, in one embodiment the cell that expresses the transcript is an industrially relevant cell, for example a S. cerevisiae cell, a Pichia pastoris cell, a Kluyveromyces lactis cell, a Yarrowia lipolytica cell, a Rhodosporidium toruloides cell a E. coli cell, a B. subtilis cell, a Cyanobacteria cell for example Synechocystis PCC 6803m or CHO cells. In a preferred embodiment the cell is a S. cerevisiae cell.

The cell may also be a medically relevant cell, for example a pathogenic cell or a cancer cell, for example the cell may be selected from the group consisting of a HEK239T cell, a CHO cell, a HeLa cell, or a T-cell. The cell also may be from, or in, a patient suffering from a disease, for example a patient that has a disease in which it is considered that entire pathways are dysregulated, for example Glioblastoma multiforme, Diabetes (type I and type II), Multiple sclerosis, Autoimmune diseases or Huntington's disease.

As mentioned previously, the type of RNA mediated gene regulation or editing that the nucleic acid sequences are mediating can be, for example siRNA or CRISPR. Some of these methods of regulation require additional factors. For example, CRISPR, CRISPRi or CRISPRa require a polypeptide that is capable of association with the sgRNA. A commonly used polypeptide is the Cas9 polypeptide. However, other Cas9 like polypeptides exist that can also mediate CRISPR type gene regulation. Accordingly, in one embodiment, where at least one of the nudeic acid sequences that directs RNA mediated gene regulation is a gRNA the method further comprises co-expressing a polypeptide capable of associating with the sgRNA, wherein the polypeptide is selected from the group consisting of:
Cas9 or Cas9-ike polypeptide, for example wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

The polypeptide may also be fused to an activation and/or repression domain, for example may be fused to an activation domain selected from the group consisting of VP, VP16, VP64, Gal4, or B42; and/or may be fused to a repression domain selected from the group consisting of KRAB-like effectors (e.g. Mxi1), RD1152, RD11, RD5 or RD2.

Such fusions are well known in the art and are the skilled person is readily able to produce the required fusion protein.

Preferences for Cas9 fusion proteins apply throughout.

The polypeptide may also be fused to an error-prone DNA polymerase to function as a site-directed mutagenesis platform. In one embodiment, such a polypeptide fusion is used in conjunction with the methods and nucleic acids described herein, for example the gRNA multiplexing platform described herein, to initiate mutations at multiple positions in the genome simultaneously. Halperin et al 2018 Nature 560: 248-252 describes methods involving the use of CRISPR-guided DNA polymerases.

In addition, the polypeptide may be used to induce double strand breaks in target nucleic acids and which, following homology-direct repair, can be used to create knockin genes as well as gene knockouts.

In this case, the nucleic acids that mediate gene regulation can have different sequences for association with different Cas9 or Cas9 like proteins, one of which may be an activating protein, and one of which may be a repressor protein, for example.

Preferences for the features described above, including but not limited to, the type of nucleic acid (DNA or RNA; linear or circular), type of gene regulation, size and number/frequency of nucleic acid fragments, position of primer hybridisation sites, cleavage sites, lining primers, cell type, promoters and destination vectors, and other features, apply equally to all aspects and embodiments described below.

In addition to the above claimed methods, it will be clear to the skilled person that the invention also provides the various components required to put the methods into practice, and the products of the methods, for example the GRRG vector and the RNA mediated gene regulating or editing nucleic acid construct that comprises at least two sequences that are transcribed into nucleic acid polymers that each separately direct RNA mediated gene regulation or editing.

Accordingly, in one embodiment, the invention provides an RNA mediated gene regulating or editing nucleic acid construct that comprises at least two sequences that are transcribed into nucleic acid polymers that each separately direct RNA mediated gene regulation or editing. Preferences for the RNA mediated gene regulating or editing nucleic acid construct and its constituent components are as described for earlier aspects and embodiments of the invention. For example, the RNA mediated gene regulating or editing nucleic acid construct may be a linear nucleic acid or may be a circular nucleic acid. Preferably the construct is circular. The construct may be of any type of nucleic acid, for example DNA or RNA. Preferably the construct is a DNA construct. The construct may comprise any number of sequences that are transcribed into nucleic acid polymers that each separately direct RNA mediated gene regulation or editing. The gene regulation may occur through for example CRISPR mediated mechanisms, or siRNA. The construct may comprise any promoter. Exemplary promoters are indicated above. The nucleic acid construct may or may not have been made in accordance with the methods described herein. However, preferably the nucleic acid construct has been made by the method of the invention. This is particularly advantageous since the present method is considered to result in each of the constituent sequences that direct RNA mediated gene regulation or editing actually being processed into active RNA polymers that affect gene expression or that can edit genes. In the prior art methods, not all of the individual RNA polymers were found to be active.

In one embodiment the invention provides an RNA mediated gene regulating or editing nucleic acid construct that comprises at least two sequences that are transcribed into nucleic acid polymers that each separately direct RNA mediated gene regulation or editing, for example wherein the construct comprises at least 11 or at least 12 nucleic acid sequences that encode an RNA mediated gene regulation or editing directing sequence.

In one embodiment the invention provides an RNA mediated gene regulating or editing nucleic acid construct that comprises at least 11 nucleic acid sequences that encode an RNA mediated gene regulation or editing directing sequence, wherein between each sequence that encodes an RNA mediated gene regulation or editing directing sequence is a sequence that when in RNA form is a cleavage site, wherein the cleavage site is selected from the group consisting of a Csy4 cleavage site, a tRNA sequence, a ribozyme sequence or an intron sequence, wherein the single nucleic acid molecule comprises a promoter capable of driving expression from the at least 11 nucleic acid sequences to form one single RNA transcript, for example wherein the single RNA molecule comprises between 11 and 100 nucleic acid sequences that encode an RNA mediated gene regulation or editing directing sequence, optionally 12 and 90 13 and 80, 14 and 70, 15 and 60, 20 and 50, 30 and 40 nucleic acid nucleic acid sequences that encode an RNA mediated gene regulation or editing directing sequence, for example wherein the single RNA molecule comprises 11 or 12 nucleic acid sequences that direct RNA mediated gene regulation or editing.

As discussed, preferably the RNA mediated gene regulating or editing nucleic acid construct of the invention is circular, for example is a circular plasmid. Also as discussed above, the RNA mediated gene regulating or editing nucleic acid construct preferably comprises exit cleavage sites which allow the ready excision of the single nucleic acid assembly which comprises the assembled amplification products (that in turn comprise the nucleic acid sequences that encode RNA mediated gene regulation or editing directing sequences) so that it can be transferred to a different vector, for example, which may have a promoter from a different species, or a different strength promoter, for example.

The skilled person will understand that the RNA mediated gene regulating or editing nucleic acid construct of the invention may be suitable for use in any organism, and the skilled person is able to identify the required components, such as promoters and terminators, that allow the construct to function in different organisms, such as yeast for example S. cerevisiae, and mammals. For example, the invention provides an RNA mediated gene regulating or editing nucleic acid construct of the invention wherein the nucleic acid construct is suitable for the expression of at least 11 nucleic acid sequences to form one single RNA transcript in eukaryotes, for example suitable for expression in mammalian cells or yeast cells or by mammalian or yeast in vitro transcription systems. Alternatively, the RNA mediated gene regulating or editing nucleic acid construct of the invention may be suitable for the expression of the at least 11 nucleic acid sequences to form one single RNA transcript in prokaryotes, for example E. coli.

In one embodiment, the RNA mediated gene regulating or editing nucleic acid construct of the invention has been constructed by the methods of the invention. In another embodiment, the RNA mediated gene regulating or editing nucleic acid construct has not been constructed by the methods of the invention.

The invention also provides a single RNA molecule that is or has been transcribed from the RNA mediated gene regulating or editing nucleic acid construct of the invention. In one embodiment the single RNA molecule comprises at least 11 nucleic acid sequences that direct RNA mediated gene regulation or editing, wherein between each nucleic acid sequence that directs RNA mediated gene regulation or editing is a sequence that is a cleavage site wherein the cleavage site is selected from the group consisting of a Csy4 cleavage site, a tRNA sequence, a ribozyme sequence or an intron sequence. For example, in one embodiment the single RNA molecule comprises between 11 and 100 nucleic acid sequences that direct RNA mediated gene regulation, optionally 12 and 90, 13 and 80, 14 and 70, 15 and 60, 20 and 50, 30 and 40, nucleic acid sequences that direct RNA mediated gene regulation or editing. For example in one embodiment the single RNA molecule comprises 11 or 12 nucleic acid sequences that direct RNA mediated gene regulation or editing. For example, in one embodiment the single RNA molecule comprises up to 6 nucleic acid sequences that are transcribed into nucleic acid polymers that each separately direct RNA mediated gene regulation, or up to 12, 18, 24, 30, 36, 42 or 48 nucleic acid sequences that are transcribed into nucleic acid polymers that each separately direct RNA mediated gene regulation.

The invention also provides a gene regulating RNA generating (GRRG) vector that comprises a selectable marker, for example a drop-out marker (in addition to an optional antibiotic selection marker for maintenance in cloning vehicles) and a nucleic acid sequence that when in RNA form comprises a cleavage site wherein the cleavage site is selected from a Csy4 cleavage site, a tRNA, a ribozyme cleavage site, or an intron. In some embodiments, the vector further comprises a scaffold sequence that when in RNA form allows association of the RNA with a polypeptide, for example a polypeptide selected from the group consisting of:
Cas9 or Cas9-like polypeptide, optionally wherein the Cas9 polypeptide is a 25 Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

In some embodiments, the polypeptide is fused to an activation and/or repression domain, for example wherein the activation domain is selected from the group consisting of VP, VP16. VP64, Gal4, or B42; and/or wherein the repression domain is selected from the group consisting of KRAB-like effectors (e.g. Mxi1), RD1152, RD11, RD5 or RD2. In some embodiments the polypeptide is fused to an error prone DNA polymerase.

In some embodiments of the GRRG vector, the vector comprises the following components in the following order 5′ to 3′:

a) nucleic acid sequence that when in RNA form comprises a Csy4 cleavage site, a tRNA, a ribozyme cleavage site or an intron

b) the selectable marker; and

c) the scaffold sequence.

The skilled person will realise that many of the uses of the nucleic acids and methods described herein require transformation of the nucleic acid into cells. Such transformation is often performed through the use of viral or phage vectors. The nucleic acid is packaged inside the virus or phage particle, and is then delivered into the cell. Accordingly, in one embodiment the invention provides a phage or viral vector that comprises the RNA mediated gene regulating or editing nucleic acid construct of the invention or the single RNA molecule of the invention that is or has been transcribed from the RNA mediated gene regulating or editing nucleic acid construct of the invention, for example wherein the phage or viral vector is selected from the group consisting of adeno-associated virus (AAV), Hybrid Adenoviral Vectors and Herpes simplex viruses The skilled person is well aware of suitable phage or viral delivery vectors.

Other delivery vehicles include bacteriophage lambda vectors and thermoresponsive bacteriophage nanocarriers.

The skilled person will understand that in some embodiments, rather than delivering the nucleic acids of the invention through the use of viral or phage delivery vectors, naked DNA can be taken up directly by the cell, or ultrasound, electroporation and cationic lipids, for example can be used to enhance uptake of the nucleic acid.

Or bacteriophage lambda vectors, thermoresponsive bacteriophage nanocarriers, etc.

The invention also provides a cell comprising the RNA mediated gene regulating or editing nucleic acid construct of the invention; or the single RNA molecule of the invention that is or has been transcribed from the RNA mediated gene regulating or editing nucleic acid construct of the invention; or the phage or viral vector of the invention. The cell can be any cell type or from any species. Preferences for the cell are as discussed herein. It should be apparent that the cell may comprise more than one RNA mediated gene regulating nucleic acid construct of the invention, for example wherein each RNA mediated gene regulating or editing nucleic acid construct of the invention comprises a different promoter, for example inducible promoters, and/or wherein the RNA mediated gene regulating or editing nucleic acid constructs of the invention are directed towards the regulation or editing of different genes, or different sets of genes. This preference is applicable to the cell and all methods of the invention.

To allow the cleavage of the single transcript into individual nucleic acids that direct gene regulation or editing, in some embodiments the cell of the invention expresses (or co-expresses), or otherwise comprises, an agent that is capable of cleaving the sequence that when in RNA form comprises a cleavage site. Preferences for the agent that is capable of cleaving the sequence that when in RNA form comprises a cleavage site are as described herein. For example where the sequence that when in RNA form is a cleavage site comprises the Csy4 cleavage site, the cell expresses or comprises a Csy4 polypeptide. In other examples, where the sequence that when in RNA form is a cleavage site comprises a tRNA sequence, the cell expresses or otherwise comprises RNase P, RNase Z and/or RNase E. In another example, where the sequence that when in RNA form is a cleavage site comprises a ribozyme cleavage site, the cell expresses or otherwise comprises the appropriate ribozyme. In a further example, where the sequence that when in RNA form is a cleavage site comprises an intron, the cell expresses or otherwise comprises native splicing machinery.

The invention also provides linker primers that, following cleavage, results in the unique BsmBI overhangs as depicted in Table 11. The linker primers of the invention may have any target sequence, i.e. sequence that is capable of hybridising to a template vector for example, along with any one of the unique 5′ sequences in Table 11.

In one embodiment the invention provides a pair of primers each with one of the unique 5′ sequences of Table 11. In another embodiment the invention provides at least 1, 2,3, 4, 5, 6, 7, 8, 9, 10, 11, or at least 12 primer pairs, each primer pair having a different set of 5′ sequences of Table 11 so that amplification products can be ligated to one another in an orderly fashion.

In one embodiment the invention provides one or more forward and reverse primers with a 5′ sequence from Table 11, in addition to a 3′ target sequence:

The skilled person will understand which primers to use to allow ligation of the amplification product to another amplification product that has been amplified using a different primer pair.

TABLE 11

| Seq ID | Forward/ | | 4bp BsmBI |
|---|---|---|---|
| NO | Reverse | 5′ primer sequence | Overhang |
| 52 | Forward | GCATCGTCTCATGCC | TGCC |
| 53 | Reverse | ATGCCGTCTCATAGT | |
| 54 | Forward | GCATCGTCTCAACTA | ACTA |
| 55 | Reverse | ATGCCGTCTCATCTG | |
| 56 | Forward | GCATCGTCTCACAGA | CAGA |
| 57 | Reverse | ATGCCGTCTCAGTAA | |
| 58 | Forward | GCATCGTCTCATTAC | TTAC |
| 59 | Reverse | ATGCCGTCTCACACA | |
| 60 | Forward | GCATCGTCTCATGTG | TGTG |
| 61 | Reverse | ATGCCGTCTCAGCTC | |
| 62 | Forward | GCATCGTCTCAGAGC | GAGC |
| 63 | Reverse | ATGCCGTCTCAGAAT | |
| 64 | Forward | GCATCGTCTCAATTC | ATTC |
| 65 | Reverse | ATGCCGICTCATTCG | |
| 66 | Forward | GCATCGTCTCACGAA | CGAA |
| 67 | Reverse | ATGCCGTCTCACGGT | |
| 68 | Forward | GCATCGTCTCAACCG | ACCG |
| 69 | Reverse | ATGCCGTCTCAAGTT | |
| 70 | Forward | GCATCGTCTCAAACT | AACT |
| 71 | Reverse | ATGCCGTCTCATCCT | |
| 72 | Forward | GCATCGTCTCAAGGA | AGGA |
| 73 | Reverse | ATGCCGTCTCATTTT | |
| 74 | Forward | GCATCGTCTCAAAAA | AAAA |
| 75 | Reverse | ATGCCGTCTCATTGC | |

As discussed above, the nucleic acid constructs and methods of the invention have a wide range of applications in any situation where there is a need for gene regulation or editing, whether activation or repression, particularly in situations where a number of different genes require regulation or editing, insertions, deletions, knockouts or knockins. For example, the invention provides a method for the regulation or editing of at least one gene in a cell wherein the method comprises any one of, or more than one of:
the method of the invention for producing an RNA mediated gene regulating or editing nucleic acid construct that comprises at least two sequences that are transcribed into nucleic acid polymers that each separately direct RNA mediated gene regulation or editing;
the method of the invention for producing at least two nucleic acid sequences that direct RNA mediated gene regulation or editing, for example at least 11 or at least 12 nucleic acid sequences that direct RNA mediated gene regulation or editing;
the use of the RNA mediated gene regulating or editing nucleic acid construct of the invention; or the single RNA molecule of the invention that is or has been transcribed from the RNA mediated gene regulating or editing nucleic acid construct of the invention
the use of the phage or viral vector according to the invention; and/or
the use of the cell according to the invention.

Preferences for features of the method for the regulation or editing of at least one gene in a cell are as described throughout the specification. For example, in one embodiment between 3 and 100 genes are regulated or editing, for example between 5 and 95 genes, and 90 genes, 15 and 85, 20 and 80, 25 and 75, 30 and 70, 35 and 65, 40 and 60, 45 and 55, for example 60 genes are regulated or editing, for example at least 11 or at least 12 genes are regulated or editing.

The gene regulation may be gene silencing, or may be gene activation. In some embodiments the regulation may be both gene silencing and activation, for example wherein a cell comprises two different RNA mediated gene regulating nucleic acid construct of the invention. In this case, the nucleic acids that mediate gene regulation can have different sequences for association with different Cas9 or Cas9 like proteins, one of which may be an activating protein, and one of which may be a repressor protein, for example. The gene editing may be to introduce deletions, inserts, knockouts or knockins. As for gene regulation, the gene editing may be of more than one type in a single cell for example, in which case association with different Cas9 proteins is required.

The invention also provides methods for the regulation or editing of at least one gene in a cell wherein the method comprises exposing the cell to the RNA mediated gene regulating or editing nucleic acid construct of the invention; or the single RNA molecule of the invention that is or has been transcribed from the RNA mediated gene regulating or editing nucleic acid construct of the invention; or the use of the phage or viral vector according to the invention. In some embodiments between 3 and 100 genes are regulated or editing, for example between 5 and 95 genes, 10 and 90 genes, 15 and 85, 20 and 80, 25 and 75, and 70, 35 and 65, 40 and 60, 45 and 55, for example 50 genes are regulated or editing, for example wherein at least 11 or at least 12 genes are regulated or editing.

Preferences for the mechanism and effect of gene regulation or editing are as described throughout the specification.

It will be immediately apparent to the skilled person that the nucleic acids that mediate the gene regulation or editing may be therapeutic nucleic acids, for example may have a role in the treatment or prevention of a disease, particularly a disease in which gene regulation of particular genes is considered to be beneficial, particularly where the regulation of a number of genes is considered to be beneficial. Accordingly, in one embodiment, the invention provides the RNA mediated gene regulating or editing nucleic acid construct of the invention; or the single RNA molecule of the invention that is or has been transcribed from the RNA mediated gene regulating or editing nucleic acid construct of the invention; or the phage or viral vector according to the invention, for use in medicine, for example for use in the treatment and/or prevention of a disease, for example for use as a vaccine. Exemplary diseases that are considered to be suitable for treatment or prevention by the present invention include diseases in which entire pathways are dysregulated, such as Glioblastoma multiforme, Diabetes (type I and type II), Multiple sclerosis, Autoimmune diseases and Huntington's disease. The invention also provides corresponding methods of treatment or prevention of disease.

The invention also provides the use of the RNA mediated gene regulating or editing nucleic acid construct of the invention; or the single RNA molecule of the invention that is or has been transcribed from the RNA mediated gene regulating or editing nucleic acid construct of the invention; or the phage or viral vector according to the invention for the manufacture of a medicament for treating or preventing disease, for example treating or preventing a disease in which entire pathways are dysregulated, such as Glioblastoma multiforme, Diabetes (type I and type II), Multiple sclerosis, Autoimmune diseases and Huntington's disease.

The invention also provides methods of therapy, wherein the method comprises administering the RNA mediated gene regulating or editing nucleic acid construct of the invention; or the single RNA molecule of the invention that is or has been transcribed from the RNA mediated gene regulating or editing nucleic acid construct of the invention; or the phage or viral vector according to the invention. Such therapies can include the treatment and/or prevention of disease, or for example for use as a vaccine. Exemplary diseases that are considered to be suitable for treatment or prevention by the present invention include diseases in which entire pathways are dysregulated, such as Glioblastoma multiforme, Diabetes (type I and type II), Multiple sclerosis, Autoimmune diseases and Huntington's disease. The invention also provides corresponding methods of treatment or prevention of disease.

The invention also has many industrial uses, for example in brewing, large-scale protein production, pharmaceutical production, metabolite production optionally the production of chemicals or fuels, biomass vs. growth or metabolic ‘valves’ (control of metabolic production/growth using inducible promoters to control regulatory RNA expression on time, e.g. after growth phase to separate growth and production, which is useful when producing toxic metabolites). Accordingly, the invention also provides, methods and uses of the nucleic acids and methods described herein for use in such purposes, for example the invention provides the RNA mediated gene regulating or editing nucleic acid construct of the invention; or the single RNA molecule of the invention that is or has been transcribed from the RNA mediated gene regulating or editing nucleic acid construct of the invention; or the phage or viral vector according to the invention for use in an industrial process, for example for use in brewing, large-scale protein production, pharmaceutical production, metabolite production optionally the production of chemicals or fuels, biomass vs. growth or metabolic ‘valves’ (control of metabolic production/growth using inducible promoters to control regulatory RNA expression on time, e.g. after growth phase to separate growth and production, which is useful when producing toxic metabolites).

The invention can also be used in lineage tracing, for example the multiplexed RNAs produced by the method can be used as a tool to trace the lineage of cells over several generations. Accordingly in one embodiment the invention provides a method of lineage tracing, wherein the method comprises the use of any of the methods or nucleic acid constructs of the invention.

The invention also provides a method of CRISPR mediated gene repression, activation or editing wherein the method comprises any one or more of:
the method of the invention for producing an RNA mediated gene regulating or editing nucleic acid construct that comprises at least two sequences that are transcribed into nucleic acid polymers that each separately direct RNA mediated gene regulation or editing;
the method of the invention for producing at least two nucleic acid sequences that direct RNA mediated gene regulation or editing, for example at least 11 or at least 12 nucleic acid sequences that direct RNA mediated gene regulation or editing;
the use of the RNA mediated gene regulating or editing nucleic acid construct of the invention; or the single RNA molecule of the invention that is or has been transcribed from the RNA mediated gene regulating or editing nucleic acid construct of the invention
the use of the phage or viral vector according to the invention; and/or
the use of the cell according to the invention.

The invention provides any of the methods disclosed herein wherein the method is performed in yeast, for example in a S. cerevisiae cell, a Pichia pastoris cell, a Kluyveromyces lactis cell, a Yarrowia lipolytica cell or a Rhodospondium toruloides cell.

There are numerous applications for nucleic acid constructs that encode RNA mediated gene regulation or editing directing sequences. For example, such a construct has uses both in industrial and medical applications.

One particular application is in the control of metabolism. For example, in one embodiment at least one, or two or more of the nucleic acid sequences that encode an RNA mediated gene regulation or editing directing sequence are directed towards genes that are involved in the control of metabolism. Some such genes from yeast include ADH, ACC1, GPD1, DGA1, HXK, ICL1, HMG1, ERG9, ERG20, ERG5, PTA, ACK, ACS2, HXT1-7, GAL2, GAPDH. Other genes from yeast and other species will be apparent to the skilled person and can be identified in the annotated sequence and organism databases.

Metabolic rewiring of target genes in vivo via transcriptional activation or repression or, optionally, deletion of these target genes can also be achieved using the nucleic acid constructs of the invention. Further uses include metabolic engineering, synthetic biology, biomaterial production, recombinant protein production, etc.

The nucleic acid constructs of the invention can also be used for the rapid deletion of genes in vivo to engineer strains with the use of fewer numbers of transformations compared to standard methods.

The invention also has applications in genome engineering. For example, multiplexed gRNAs can be used to cleave genomic DNA fragments and move them between organisms for numerous applications in genome synthesis (see Wang et al 2016 Nature 539: 59-64).

The invention also has applications in RNA detection with CRISPR-Cas13a/C2c2, for example by multiplexing gRNAs many viruses can be detected/cleaved simultaneously, for example on paper-based diagnostics.

Preferences for the features described above, including but not limited to, the type of nucleic acid (DNA or RNA; linear or circular), type of gene regulation, size and number/frequency of nucleic acid fragments, position of primer hybridisation sites, cleavage sites, lining primers, cell type, promoters and destination vectors, and other features, apply equally to all aspects and embodiments described below.

The skilled person will understand that the methods of the invention lend themselves readily to the components parts being provided as a kit, or a kit of parts. Accordingly, the invention provides a kit or kit of parts comprising any of the components discussed herein. For example, the invention provides a kit comprising any two or more of:

i) a GRRG vector according to the invention, for example a gene regulating RNA generating (GRRG) vector, wherein the GRRG vector comprises a selectable marker nucleic acid sequence and a nucleic acid sequence that when in RNA form comprises a cleavage site, optionally wherein the cleavage site is selected from:
a) an endoribonuclease cleavage site, for example a site-specific RNA endonuclease site, for example an artificial site-specific RNA endonucleases or a Csy4 cleavage sequence
b) a tRNA sequence
c) a ribozyme sequence
d) an intron
e) a target sequence for an RNA directed cleavage complex

optionally wherein the GRRG vector further comprises a scaffold sequence that when in RNA form allows association of the RNA with a polypeptide capable of regulating or editing a gene. In one embodiment the polypeptide is selected from the group consisting of:
Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida);
optionally wherein the polypeptide is fused to an activation and/or repression domain, optionally
wherein the activation domain is selected from the group consisting of VP, VP16, VP64, Gal4, or B42; and/or
wherein the repression domain is selected from the group consisting of KRAB-like effectors (e.g. Mxi1), RD1152, RD11, RD5 or RD2

optionally wherein the GRRG comprises the following components in the following order 5′ to 3′:
a) nucleic acid sequence that when in RNA form comprises a Csy4 cleavage site, a tRNA, a ribozyme cleavage site, an intron, or a target sequence for an RNA directed cleavage complex
b) the selectable marker; and
c) the scaffold sequence;

ii) a GRRG forward and reverse primer according to the invention

iii) one or more linking primer pairs according to the invention

iv) a destination vector according to the invention

v) a nucleic acid encoding a polypeptide selected from the group consisting of Cas9, optionally

wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida), optionally wherein the polypeptide is fused to an activator or repressor domain, or an error-prone DNA polymerase

vi) one or more Type II S restriction enzymes, optionally BsmBI;

vii) a nucleic acid encoding a Csy4 polypeptide, optionally wherein the nucleic acid is a circular vector;

vii) one or more restriction enzymes

ix) DNA polymerase

x) DNA ligase

xi) one or more intermediate vectors.

In one embodiment the kit comprises the gene regulating RNA generating vector of the invention and any one or more of the additional elements (ii) to (x).

It ought to be clear to the skilled person that a single RNA mediated gene regulating or editing nucleic acid construct of the invention may comprise sequences that have been amplified from different GRRG template vectors. Such an embodiment may be useful if, for example, the GRRG vectors comprise different Cas9 or Cas9 like scaffold sequences. This would allow some of the RNA polymers that direct gene regulation or editing to associate with one Cas9 or Cas9 like polypeptide, whilst one or more of the other RNA polymers that direct gene regulation or editing may associate with a different Cas9 or Cas9 like polypeptide. The different Cas9 or Cas9 like polypeptides may be fused to, for example, an activator domain and a repressor domain. In this instance, multiple RNA polymers that direct gene regulation can be expressed from a single nucleic acid, yet some may be gene activating and some may be gene regulating.

As indicated here, the method for producing an RNA mediated gene regulating or editing nucleic acid construct that comprises at least two sequences that are transcribed into nucleic acid polymers that each separately direct RNA mediated gene regulation described herein can actually be used to produce a nucleic acid that generates transcripts that have functions other than in RNA mediated gene regulation. For example the method of the invention can be used to combine and assemble sequences that are useful for DNA origami or RNA origami. In these instances, the name given to the GRRG is not entirely accurate, since the vector is not for generating RNA polymers that regulate gene expression or editing, but is rather for generating RNA polymers that are useful in DNA origami or RNA origami. In this instance, a preferred name for the GRRG would be, for example an RNA for Origami Generating vector, for example an ROG vector. Preferences for the ROG vector are largely the same as for the GRRG vector, other than a scaffold sequence is likely not required, and the forward GRRG primer (again, which in this instance would be renamed as the forward RNA for origami nucleic acid generating primer) would comprise at the 5′ end a sequence that encodes a nucleic acid that is useful in DNA or RNA origami rather than the sequence that encodes an RNA polymer that directs RNA mediated gene regulation or editing.

Nucleic acids for use in DNA origami are often are made with several short DNA or RNA molecules which usually contain repeated domains and therefore cannot be synthetized in a single molecule easily. The methods of the present invention would make it possible to generate long RNAs with repeated domains that could fold in the desired manner and generate the designed patterns/structures. In addition to RNA origami, DNA origami could be also generated from the destination vector after treating it with a nuclease that converts the dsDNA into ssDNA, which could fold in DNA origami.

Accordingly, the invention also provides a method of performing DNA origami wherein the method comprises:
the method for producing an RNA mediated gene regulating or editing nucleic acid construct wherein the method has been adapted for the production of nucleic acids useful in DNA origami as discussed above
the method for producing at least two nucleic acid sequences that direct RNA mediated gene regulation or editing wherein the method has been adapted for the production of nucleic acids useful in DNA origami as discussed above
the use of the RNA mediated gene regulating or editing nucleic acid construct of the invention wherein the construct has been adapted for the production of nucleic acids useful in DNA origami as discussed above;
the single RNA molecule of the invention wherein the single RNA molecule has been adapted for the production of nucleic acids useful in DNA origami as discussed above
the use of the phage or viral vector according to the invention that comprises the RNA mediated gene regulating nucleic acid construct of the invention wherein the construct has been adapted for the production of nucleic acids useful in DNA origami as discussed above or that comprises the single RNA molecule of the invention wherein the single RNA molecule has been adapted for the production of nucleic acids useful in DNA origami as discussed above and/or
the use of the cell according to the invention that comprises
a) the RNA mediated gene regulating or editing nucleic acid construct of the invention wherein the construct has been adapted for the production of nucleic acids useful in DNA origami as discussed above;
b) the single RNA molecule of the invention wherein the single RNA molecule has been adapted for the production of nucleic acids useful in DNA origami as discussed above or
c) the phage or viral vector according to the invention that comprises the RNA mediated gene regulating nucleic acid construct of the invention wherein the construct has been adapted for the production of nucleic acids useful in DNA origami as discussed above or that comprises the single RNA molecule of the invention wherein the single RNA molecule has been adapted for the production of nucleic acids useful in DNA origami as discussed above.

For example, the invention provides:

a method for producing a DNA or RNA origami nucleic acid generating construct that comprises at least two sequences that are transcribed into nucleic acid polymers that each separately are useful in DNA or RNA origami, wherein the at least two nucleic acid sequences are transcribed into a single transcript from a single promoter, wherein the method comprises:

a) amplifying a cassette from an RNA for Origami Generating vector (ROG vector) using at least two ROG primer pairs, each ROG primer pair comprising a forward and a reverse primer,
wherein the ROG vector comprises a selectable marker nucleic acid sequence and a nucleic acid sequence that when in RNA form comprises a cleavage site, optionally wherein the cleavage site is selected from:
i) an endoribonuclease cleavage site, for example a site-specific RNA endonuclease site, for example an artificial site-specific RNA endonucleases or a Csy4 cleavage sequence
ii) a tRNA sequence
iii) a ribozyme sequence
iv) an intron
v) a target sequence for an RNA directed cleavage complex
wherein the forward and reverse ROG primers comprise nucleic acid sequences that are complementary to sequences of the ROG vector and allow hybridisation of the primers to the ROG vector at either side of the selectable marker sequence such that upon hybridisation the primers are directed away from the selectable marker nucleic acid sequence,
wherein the reverse ROG primer hybridises to a common portion of the sequence that when in RNA form comprises a cleavage site, optionally wherein the sequence of the reverse primer is the same for each reverse primer in each primer pair, and wherein the forward ROG primer hybridises to a common forward primer hybridisation sequence of the ROG vector,
wherein the forward ROG primer further comprises a sequence that encodes an RNA polymer that is useful in DNA or RNA origami, which is not complementary to the vector nucleic acid sequence and which is located 5′ of the forward primer sequence that is complementary to the ROG vector
wherein amplification using each of the forward and reverse ROG primer pairs results in the production of a linear cassette that comprises the following components in the following order 5′ to 3′:
i) the sequence that encodes an RNA useful in DNA or RNA origami
ii) the forward primer hybridisation sequence
iii) the nucleic acid sequence that when in RNA form comprises a cleavage site
but which does not comprise the marker nucleic acid sequence,
optionally wherein the linear cassette comprising intervening nucleic acid located between (ii) the forward primer hybridisation sequence and (iii) the nucleic acid sequence that when in RNA form comprises a cleavage site; and

b) separately re-circularising each of the linear cassettes produced in step (a) to produce a circular nucleic acid polymer such that the sequence that encodes an RNA polymer useful in DNA or RNA origami, is located between the forward primer hybridisation sequence and the nucleic acid sequence that when in RNA form comprises a cleavage site; and

c) providing at least two linking primer pairs, each primer pair comprising
wherein the forward linking primer is capable of hybridising to the nucleic acid sequence that when in RNA form comprises a cleavage site and the reverse linking primer is capable of hybridising to the common forward primer hybridisation sequence of the RMG vector,
wherein each of the forward and reverse linking primers comprises a nucleic acid sequence capable of forming a single-stranded overhang, optionally wherein each primer comprises a Type II S restriction site, wherein each pair of forward and reverse linking primers are designed so that following amplification the single-stranded overhang generated at one end of the amplification product generated by a first linking primer pair is able to hybridise with a compatible single-stranded overhang generated at one end of a second amplification product generated by a second linking primer pair; and

d) amplifying each of the cassettes formed in step (b) with the appropriate pair of linking primers of (c); and

e) treating the amplification products of (d) to generate a single-stranded overhang, optionally digesting the amplification products with an appropriate Type II S restriction enzyme(s); and

f) assembling the treated amplification products of (e) to one another to generate a single nucleic acid assembly comprising the assembled amplification products; and

g) ligating the single nucleic acid of (f) to a nucleic acid comprising a promoter sequence and optionally a terminator sequence,
optionally wherein the promoter nucleic acid sequence and/or optional terminator sequence has compatible overhangs to the ends of the single nucleic acid of (f), such that the promoter is located 5′ to the ligated amplification products of (f) and is capable of driving expression of a single transcript from the ligated amplification products and the optional terminator is located 3 to the ligated amplification products of (f)

optionally where steps (f) and (g) are performed simultaneously; or

(h)(i) ligating the single nucleic acid of (f) to an intermediate nucleic acid vector producing an intermediate vector comprising the single nucleic acid assembly of step (f), optionally where steps (f) and (h) are performed simultaneously;
(ii) performing steps (a) to (f) and (h)(i) at least twice resulting in at least two different intermediate vectors each comprising a different single nucleic acid assembly of step (f);
(iii) digesting the respective at least two intermediate vectors to produce at least two cleavage fragments comprising different nucleic acid assemblies; and/or amplifying the at least two different nucleic acid assemblies from the at least two intermediate vectors;
(iv) ligating the at least two cleavage fragments or the at least two amplification products into a single destination or expression vector producing an array of nucleic acid assemblies of (f),
wherein the destination or expression vector comprises a promoter and optionally a terminator, wherein the promoter is located 5′ to the array of nucleic acid assemblies of (f) and is capable of driving expression of a single transcript from the array, and the optional terminator is located 3′ to the array of nucleic acid assemblies of (f).

A further use of the present methods and nucleic acids is in the production of polypeptides that comprise tandem arrays of repetitive sequence motifs. In this instance, the GRRG (which in this case is better referred to as a repetitive motif generating vector, or RMG vector) may in some or all embodiments not comprise a nucleic acid sequence that when in RNA form comprises a cleavage site, wherein the cleavage site, since the aim of this method would be to build up a series of motifs that are expressed as a single transcript which is then translated into a single polypeptide. In this aspect, the forward GRRG primer (again, which in this instance would be renamed as the forward repetitive motif generating primer) would comprise at least part of the repetitive sequence motif. For example, the forward primer could not have a 5′ tail region and be fully complementary to a region of the RMG vector which comprises the repeat motif. Alternatively, the forward primer can have a tail sequence which can be used to introduce variation into the repeat sequence motifs

The invention also provides:

a method for producing a nucleic acid construct that encodes a polypeptide wherein the polypeptide comprises tandem arrays of repetitive sequence motifs

wherein the method comprises:

a) amplifying a cassette from a repetitive motif generating vector (RMG vector) using at one or more optionally at least two RMG primer pairs, each RMG primer pair comprising a forward and a reverse primer,
wherein the RMG vector comprises a selectable marker nucleic acid sequence and a sequence encoding a repetitive motif and a nucleic acid sequence that when in RNA form comprises a cleavage site, wherein the cleavage site is selected from:
i) a Csy4 cleavage sequence
ii) a tRNA sequence
iii) a ribozyme sequence
iv) an intron
wherein the forward and reverse RMG primers comprise nucleic acid sequences that are complementary to sequences of the RMG vector and allow hybridisation of the primers to the RMG vector at either side of the selectable marker sequence such that upon hybridisation the primers are directed away from the selectable marker nucleic acid sequence,
wherein the reverse RMG primer hybridises to a common portion of the sequence that when in RNA form comprises a cleavage site, optionally wherein the sequence of the reverse primer is the same for each reverse primer in each primer pair, and wherein the forward RMG primer hybridises to a common forward primer hybridisation sequence of the RMG vector,
wherein the forward RMG primer optionally further comprises a sequence which is not complementary to the vector nucleic acid sequence and which is located 5′ of the forward primer sequence that is complementary to the RMG vector
wherein amplification using each of the forward and reverse RMG primer pairs results in the production of a linear cassette that comprises the following components in the following order 5′ to 3′:
i) the optional 5′ tail sequence
ii) the forward primer hybridisation sequence
iii) the sequence encoding a repetitive motif
iii) the reverse primer hybridisation sequence
but which does not comprise the marker nucleic acid sequence; and

b) separately circularising each of the linear cassettes produced in step (a) to produce a circular nucleic acid polymer such that the sequence that encodes a repetitive motif is located between the forward primer hybridisation sequence and the reverse primer hybridisation sequence; and

c) providing at least two linking primer pairs
wherein the forward linking primer is capable of hybridising to the reverse primer hybridisation sequence of the RMG and the reverse linking primer is capable of hybridising to the forward primer hybridisation sequence of the RMG vector,
wherein each of the forward and reverse linking primers comprises a nucleic acid sequence capable of forming a single-stranded overhang, optionally wherein each primer comprises a Type II S restriction site or a homing endonuclease site, wherein each pair of forward and reverse linking primers are designed so that following amplification the single-stranded overhang generated at one end of the amplification product generated by a first linking primer pair is able to hybridise with a compatible single-stranded overhang generated at one end of a second amplification product generated by a second linking primer pair; and

d) amplifying each of the cassettes formed in step (b) with the appropriate pair of linking primers of (c); and

e) treating the amplification products of (d) to generate a single-stranded overhang, optionally digesting the amplification products with an appropriate Type II S restriction enzyme(s) or homing endonuclease; and

f) assembling the treated amplification products of (e) to one another to generate a single nucleic acid assembly comprising the assembled amplification products; and

g) ligating the single nucleic acid of (f) to a nucleic acid comprising a promoter sequence and optionally a terminator sequence,
optionally wherein the promoter nucleic acid sequence and/or optional terminator sequence has compatible overhangs to the ends of the single nucleic acid of (f), such that the promoter is located 5′ to the ligated amplification products of (f) and is capable of driving expression of a single transcript from the ligated amplification products and the

optional terminator is located 3′ to the ligated amplification products of (f) optionally where steps (f) and (g) are performed simultaneously; or

(h)(i) ligating the single nucleic acid of (f) to an intermediate nucleic acid vector producing an intermediate vector comprising the single nucleic acid assembly of step (f), optionally where steps (f) and (h) are performed simultaneously;
(ii) performing steps (a) to (f) and (h)(i) at least twice resulting in at least two different intermediate vectors each comprising a different single nucleic acid assembly of step (f);
(iii) digesting the respective at least two intermediate vectors to produce at least two cleavage fragments comprising different nucleic acid assemblies; and/or amplifying the at least two different nucleic acid assemblies from the at least two intermediate vectors;
(iv) ligating the at least two cleavage fragments or the at least two amplification products into a single destination or expression vector producing an array of nucleic acid assemblies of (f),
wherein the destination or expression vector comprises a promoter and optionally a terminator, wherein the promoter is located 5′ to the array of nucleic acid assemblies of (f) and is capable of driving expression of a single transcript from the array, and the optional terminator is located 3′ to the array of nucleic acid assemblies of (f).

All methods, primers, nucleic acid constructs and other components discussed above in relation to RNA mediated gene regulation or editing are also all specifically and explicitly considered part of the invention in the context of DNA or RNA origami or in the context of the production of polypeptides that comprise tandem arrays of repetitive sequence motifs. Preferences for the features described in relation to the earlier aspects and embodiments that relate to gene regulation or editing apply equally to the use in DNA/RNA origami or production of polypeptides that comprise tandem arrays of repetitive sequence motifs. For example including, but not limited to, the type of nucleic acid (DNA or RNA; linear or circular), type of gene regulation, size and number/frequency of nucleic acid fragments, position of primer hybridisation sites, cleavage sites, lining primers, cell type, promoters and destination vectors, and other features, apply equally to all aspects and embodiments described below.

The listing or discussion of an apparently prior-published document in this specification should not necessarily be taken as an acknowledgement that the document is part of the state of the art or is common general knowledge.

It should be apparent that preferences and options for a given aspect, feature or parameter of the invention should, unless the context indicates otherwise, be regarded as having been disclosed in combination with any and all preferences and options for all other aspects, features and parameters of the invention. For example, the invention provides a method for producing a RNA mediated gene regulating nucleic acid construct that is a linear DNA construct that comprises 24 sequences that are transcribed into gRNA sequences, wherein the construct comprises a Csy4 cleavage site and a Cas9 scaffold sequence and a LacZ marker.

TABLE 2
Sequences disclosed herein:

| Seq | | |
|---|---|---|
| ID | Sequence | Details |
| 1 | GTTCACTGCCGTATAGGCAG | 20 nucleotide DNA |
| 2 | GTTCACTGCCGTATAGGCAGCTAAGAAA | sequence encoding |
| | | the Csy4 site |
| | | 28 nucleotide DNA |
| | | sequence encoding |
| | | the Csy4 site |
| 3 | GUUCACUGCCGUAUAGGCAG | 20 Csy4 RNA |
| 4 | GUUCACUGCCGUAUAGGCAGCUAAGAAA | sequence |
| | | 28 Csy4 RNA |
| | | sequence |
| 5 | AACAAAGCACCAGTGGTCTAGTGGTAGAATAGTACCCTGCCACG | pre-tRNA^{Gly} |
| | GTACAGACCCGGGTTCGATTCCCGGCTGGTGCA | |
| 6 | gtgaGCATTGGTGGTTCAGTGGTAGAATTCTCGCCTGCCACGCGG | Dr-tRNAGly(GCC) |
| | GAGGCCCGGGTTCGATTCCCGGCCAATGCA | |
| 7 | gttCtcatcaGCCCGGCTAGCTCAGTCGGTAGAGCATGAGACTCTTA | Dr-tRNALys(CTT) |
| | ATCTCAGGGTCGTGGGTTCGAGCCCCACGTCGGGCG | |
| 8 | gctatctGTCTCTGTGGCGCAATCGGTTAGCGCGTTCGGCTGTTAA | Dr-tRNAAsn(GTT) |
| | CCGAAAGGTTGGTGGTTCGAGCCCACCCAGGGACG | |
| 9 | gcctgaagGTTTCCGTAGTGTAGTGGTTATCACGTTCGCCTCATAC | Dr-tRNAMet(CAT) |
| | GCGAAAGGTCCCCAGTTCGAAACTGGGCGGAAACA | |
| 10 | gacttgaGGTTCCATGGTGTAATGGTTAGCACTCTGGACTCTGAAT | Dr-tRNAGln(CTG) |
| | CCAGCGATCCGAGTTCAAATCTCGGTGGGACCA | |
| 11 | ggaaaatGACGAGGTGGCCGAGTGGTTAAGGCGATGGACTGCTAA | Dr-tRNASer(GCT) |
| | TCCATTGTGCTTTGCACGCATGGGTTCGAATCCCATCCTCGTCG | |
| 12 | gcagcGGCGCCGTGGCTTAGTTGGTTAAAGCGCCTGTCTAGTAAA | Dr-tRNAThr(AGT) |
| | CAGGAGATCCTGGGTTCGAATCCCAGCGGTGCCT | |
| 13 | gctcGCCGTGATCGTACAGTGGTTAGTACTCTGCGTTGTGGCCGC | Dr-tRNAHis(GTG) |
| | AGCAACCCCGGTTCGAATCCGGGTCACGGCA | |
| 14 | gcatGTCAGGATGGCCGAGTGGTCTAAGGCGCTGCGTTCAGGTC | Dr-tRNALeu(CAG) |
| | GCAGTCTCCCCTGGAGGCGTGGGTTCGAATCCCACTTCTGACA | |
| 15 | gaacaaaGCACCAGTGGTCTAGTGGTAGAATAGTACCCTGCCACG | Os-tRNAGly(GCC) |
| | GTACAGACCCGGGTTCGATTCCCGGCTGGTGCA | Shiraki and |
| | | Kawakami 2 |
| 16 | GAACCTCTTACACGCGCAGATCAACTAAATGTACACTGCGACGG | Os-tRNAGly(GCC)- |
| | TCCGTGGCTCCGAGAGGGGTTACAGGGTACGCTG | scrambled |
| 17 | GCGCTGTGGCGTACCGGGTACGTACTCGCTTGACTGGGTTGGT | Dr-tRNAGly(GCC)- |
| | ACTAGGCGAAACCAGCTCCGTGGGATTGCACC | scrambled |
| 18 | gttccccCTGATGAGTCCGTGAGGACGAAACGAGTAAGCTCGTC | Hammerhead |
| | | ribozyme (HH) |
| 19 | GGCCGGCATGGTCCCAGCCTCCTCGCTGGCGCGGCTGGGCA | Hepatitis delta virus |
| | ACATGCTTCGGCATGGCGAATGGGAC | ribozyme (HDV) |
| 20 | GTACGCTGCTTCTCCTCTCCTCGCTTCGTTT | intron sequence |
| | CGATTCGATTTCGGACGGGTGAGGTTGTTTTGTTGCTAGATCCG | (underline = |
| | ATTGGTGGTTAGGGTTGTCGATGTGATTATCGTGAGATGTTTAG | splicing donor; bold = |
| | GGGTTGTAGATCTGATGGTTGTGATTTGGGCACGGTTGGTTCGA | branch site; italic = |
| | TAGGTGGAATCGTGGTTAGGTTTTGGGATTGGATGTTGGTTCTG | acceptor site) |
| | ATGATTGGGGGGAATTTTTACGGTTAGATGAATTGTTGGATGATT | |
| | CGATTGGGGAAATCGGTGTAGATCTGTTGGGGAATTGTGGAACT | |
| | AGTCATGCCTGAGTGATTGGTGCGATTTGTAGCGTGTTCCATCT | |
| | TGTAGGCCTTGTTGCGAGCATGTTCAGATCTACTGTTCCGCTCT | |
| | TGATTGAGTTATTGGTGCCATGGGTTGGTGCAAACACAGGCTTT | |
| | AATATGTTATATCTGTTTTGTGTTTGATGTAGATCTGTAGGGTAG | |
| | TTCTTCTTAGACATGGTTCAATTATGTAGCTTGTGCGTTTCGATT | |
| | TGATTTCATATGTTCACAGATTAGATAATGATGAACTCTTTTAATT | |
| | AATTGTCAATGGTAAATAGGAAGTCTTGTCGCTATATCTGTCATA | |
| | ATGATCTCATGTTACTATCTGCCAGTAATTTATGCTAAGAACTAT | |
| | ATTAGAATATCATGTTACAATCTGTAGTAATATCATGTTACAATCT | |
| | GTAGTTCATCTATATAATCTATTGTGGTAATTTCTTTTTACTATCT | |
| | GTGTGAAGATTATTGCCACTAGTTCATTCTACTTATTTCTGAAGT | |
| | TCAGGATACGTGTGCTGTTACTACCTATCTGAATACATGTGTGAT | |
| | GTGCCTGTTACTATCTTTTTGAATACATGTATGTTCTGTTGGAAT | |
| | ATGTTTGCTGTTTGATCCGTTGTTGTGTCCTTAATCTTGTGCTAG | |
| | TTCTTACCCTATCTGTTTGGTGATTATTTCTTGCAG | |
| 21 | CCGGCCUGUUCCCUGAGACCUCAAGUGUGAGUGUACUAUUGA | Example miRNA |
| | UGCUUCACACCUGGGCUCUCCGGGUACCAGGACGG | sequence |
| 22 | CTTCAGTGATGACACGATGACGAGTCAGAAAGGTCACGTCCTGC | Example snoRNA |
| | TCTTGGTCCTTGTCAGTGCCATGTTCTGTGGTGCTGTGCACGAG | sequence |
| | TTCCTTTGGCAGAAGTGTCCTATTTATTGATCGATTTAGAGGCAT | |
| | TTGTCTGAGAAGG | |
| 23 | NNNNNNNNNNNNNNNNNNNNgttttagagctagaaatagcaagttaaaataag | Forward Primer with |
| | | Overhang, Where N |
| | | denotes a gRNA |
| | | Target sequence |
| 24 | Phos-ctgcctatacggcagtgaac | Reverse Primer |
| | | with Overhang, |
| | | where Phos |
| | | denotes a |
| | | phosphate group |
| 25 | CTCACATGTTCTTTCCTGCG | Forward Primer for |
| | | sequencing of |
| | | fragments after |
| | | Round 1 PCR and |
| | | isolation |
| 26 | GCATCGTCTCATGCCgttcactgccgtataggcag | Forward primer |
| 27 | ATGCCGTCTCATAGTaaaagcaccgactcggtg | Reverse primer |
| 28 | GCATCGTCTCAACTAgttcactgccgtataggcag | Forward primer |
| 29 | ATGCCGTCTCATCTGaaaagcaccgactcggtg | Reverse primer |
| 30 | GCATCGTCTCACAGAgttcactgccgtataggcag | Forward primer |
| 31 | ATGCCGTCTCAGTAAaaaagcaccgactcggtg | Reverse primer |
| 32 | GCATCGTCTCATTACgttcactgccgtataggcag | Forward primer |
| 33 | ATGCCGTCTCACACAaaaagcaccgactcggtg | Reverse primer |
| 34 | GCATCGTCTCATGTGgttcactgccgtataggcag | Forward primer |
| 35 | ATGCCGTCTCAGCTCaaaagcaccgactcggtg | Reverse primer |
| 36 | GCATCGTCTCAGAGCgttcactgccgtataggcag | Forward primer |
| 37 | ATGCCGTCTCAGAATaaaagcaccgactcggtg | Reverse primer |
| 38 | GCATCGTCTCAATTCgttcactgccgtataggcag | Forward primer |
| 39 | ATGCCGTCTCATTCGaaaagcaccgactcggtg | Reverse primer |
| 40 | GCATCGTCTCACGAAgttcactgccgtataggcag | Forward primer |
| 41 | ATGCCGTCTCACGGTaaaagcaccgactcggtg | Reverse primer |
| 42 | GCATCGTCTCAACCGgttcactccgtataggcag | Forward primer |
| 43 | ATGCCGTCTCAAGTTaaaagcaccgactcggtg | Reverse primer |
| 44 | GCATCGTCTCAAACTgttcactgccgtataggcag | Forward primer |
| 45 | ATGCCGTCTCATCCTaaaagcaccgactcggtg | Reverse primer |
| 46 | GCATCGTCTCAAGGAgttcactgccgtataggcag | Forward primer |
| 47 | ATGCCGTCTCATTTTaaaagcaccgactcggtg | Reverse primer |
| 48 | GCATCGTCTCAAAAAgttcactgccgtataggcag | Forward primer |
| 49 | ATGCCGTCTCATTGCaaaagcaccgactcggtg | Reverse primer |
| 50 | gacggtaggtattgattgtaattc | Forward Prime |
| | | (binds pTDH3) |
| 51 | tgcttaatcttgtcttggctta | Reverse Primer |
| | | (binds tTDH1) |
| 52 | GCATCGTCTCATGCC | Forward |
| 53 | ATGCCGTCTCATAGT | Reverse |
| 54 | GCATCGTCTCAACTA | Forward |
| 55 | ATGCCGTCTCATCTG | Reverse |
| 56 | GCATCGTCTCACAGA | Forward |
| 57 | ATGCCGTCTCAGTAA | Reverse |
| 58 | GCATCGTCTCATTAC | Forward |
| 59 | ATGCCGTCTCACACA | Reverse |
| 60 | GCATCGTCTCATGTG | Forward |
| 61 | ATGCCGTCTCAGCTC | Reverse |
| 62 | GCATCGTCTCAGAGC | Forward |
| 63 | ATGCCGTCTCAGAAT | Reverse |
| 64 | GCATCGTCTCAATTC | Forward |
| 65 | ATGCCGTCTCATTCG | Reverse |
| 66 | GCATCGTCTCACGAA | Forward |
| 67 | ATGCCGTCTCACGGT | Reverse |
| 68 | GCATCGTCTCAACCG | Forward |
| 69 | ATGCCGTCTCAAGTT | Reverse |
| 70 | GCATCGTCTCAAACT | Forward |
| 71 | ATGCCGTCTCATCCT | Reverse |
| 72 | GCATCGTCTCAAGGA | Forward |
| 73 | ATGCCGTCTCATTTT | Reverse |
| 74 | GCATCGTCTCAAAAA | Forward |
| 75 | ATGCCGTCTCATTGC | Reverse |
| 76 | AAAGTTGGAACCTCTTACGTGCCCGATCAATCATGACCAAAATCCCTTAACGTGA | Intermediate vector |
| | GTTTTCGTTCCACTGAGCGTCAGACCCCGTAGAAAAGATCAAAGGATCTTCTTGA | 1 (psl040-1^{st}- |
| | GATCCTTTTTTTCTGCGCGTAATCTGCTGCTTGCAAACAAAAAAACCACCGCTACC | acceptor vector for |
| | AGCGGTGGTTTGTTTGCCGGATCAAGAGCTACCAACTCTTTTTCCGAAGGTAACT | up to 6 grnas |
| | GGCTTCAGCAGAGCGCAGATACCAAATACTGTTCTTCTAGTGTAGCCGTAGTTAG | |
| | GCCACCACTTCAAGAACTCTGTAGCACCGCCTACATACCTCGCTCTGCTAATCCTG | |
| | TTACCAGTGGCTGCTGCCAGTGGCGATAAGTCGTGTCTTACCGGGTTGGACTCAA | |
| | GACGATAGTTACCGGATAAGGCGCAGCGGTCGGGCTGAACGGGGGGTTCGTGC | |
| | ACACAGCCCAGCTTGGAGCGAACGACCTACACCGAACTGAGATACCTACAGCGT | |
| | GAGCTATGAGAAAGCGCCACGCTTCCCGAAGGGAGAAAGGCGGACAGGTATCC | |
| | GGTAAGCGGCAGGGTCGGAACAGGAGAGCGCACGAGGGAGCTTCCAGGGGGA | |
| | AACGCCTGGTATCTTTATAGTCCTGTCGGGTTTCGCCACCTCTGACTTGAGCGTCG | |
| | ATTTTTGTGATGCTCGTCAGGGGGGGCCAGCAACGCGGCCTTTTTACGGTTCCTG | |
| | GCCTTTTGCTGGCCTTTTGCTCACATGTTCTTTCCTGCGTTATCCCCTGATTCTGTG | |
| | GATAACCGTAGGGTCTCATTCTCTGCCGAGACGGAAAGTGAAACGTGATTTCAT | |
| | GCGTCATTTTGAACATTTTGTAAATCTTATTTAATAATGTGTGCGGCAATTCACAT | |
| | TTAATTTATGAATGTTTTCTTAACATCGCGGCAACTCAAGAAACGGCAGGTTCGG | |
| | ATCTTAGCTACTAGAGAAAGAGGAGAAATACTAGATGCGTAAAGGCGAAGAGCT | |
| | GTTCACTGGTGTCGTCCCTATTCTGGTGGAACTGGATGGTGATGTCAACGGTCAT | |
| | AAGTTTTCCGTGCGTGGCGAGGGTGAAGGTGACGCAACTAATGGTAAACTGACG | |
| | CTGAAGTTCATCTGTACTACTGGTAAACTGCCGGTTCCTTGGCCGACTCTGGTAA | |
| | CGACGCTGACTTATGGTGTTCAGTGCTTTGCTCGTTATCCGGACCATATGAAGCA | |
| | GCATGACTTCTTCAAGTCCGCCATGCCGGAAGGCTATGTGCAGGAACGCACGAT | |
| | TTCCTTTAAGGATGACGGCACGTACAAAACGCGTGCGGAAGTGAAATTTGAAGG | |
| | CGATACCCTGGTAAACCGCATTGAGCTGAAAGGCATTGACTTTAAAGAGGACGG | |
| | CAATATCCTGGGCCATAAGCTGGAATACAATTTTAACAGCCACAATGTTTACATC | |
| | ACCGCCGATAAACAAAAAAATGGCATTAAAGCGAATTTTAAAATTCGCCACAACG | |
| | TGGAGGATGGCAGCGTGCAGCTGGCTGATCACTACCAGCAAAACACTCCAATCG | |
| | GTGATGGTCCTGTTCTGCTGCCAGACAATCACTATCTGAGCACGCAAAGCGTTCT | |
| | GTCTAAAGATCCGAACGAGAAACGCGATCATATGGTTCTGCTGGAGTTCGTAAC | |
| | CGCAGCGGGCATCACGCATGGTATGGATGAACTGTACAAATGACCAGGCATCAA | |
| | ATAAAACGAAAGGCTCAGTCGAAAGACTGGGCCTTTCGTTTTATCTGTTGTTTGT | |
| | CGGTGAACGCTCTCTACTAGAGTCACACTGGCTCACCTTCGGGTGGGCCTTTCTG | |
| | CGTTTATACGTCTCTATCCTGCCTGAGACCAGACCAATAAAAAACGCCCGGCGGC | |
| | AACCGAGCGTTCTGAACAAATCCAGATGGAGTTCTGAGGTCATTACTGGATCTAT | |
| | CAACAGGAGTCCAAGCGAGCTCGATATCAAATTACGCCCCGCCCTGCCACTCATC | |
| | GCAGTACTGTTGTAATTCATTAAGCATTCTGCCGACATGGAAGCCATCACAAACG | |
| | GCATGATGAACCTGAATCGCCAGCGGCATCAGCACCTTGTCGCCTTGCGTATAAT | |
| | ATTTGCCCATGGTGAAAACGGGGGCGAAGAAGTTGTCCATATTGGCCACGTTTA | |
| | AATCAAAACTGGTGAAACTCACCCAGGGATTGGCTGAAACGAAAAACATATTCTC | |
| | AATAAACCCTTTAGGGAAATAGGCCAGGTTTTCACCGTAACACGCCACATCTTGC | |
| | GAATATATGTGTAGAAACTGCCGGAAATCGTCGTGGTATTCACTCCAGAGCGAT | |
| | GAAAACGTTTCAGTTTGCTCATGGAAAACGGTGTAACAAGGGTGAACACTATCC | |
| | CATATCACCAGCTCACCGTCTTTCATTGCCATACGAAATTCCGGATGAGCATTCAT | |
| | CAGGCGGGCAAGAATGTGAATAAAGGCCGGATAAAACTTGTGCTTATTTTTCTTT | |
| | ACGGTCTTTAAAAAGGCCGTAATATCCAGCTGAACGGTCTGGTTATAGGTACATT | |
| | GAGCAACTGACTGAAATGCCTCAAAATGTTCTTTACGATGCCATTGGGATATATC | |
| | AACGGTGGTATATCCAGTGATTTTTTTCTCCATTTTAGCTTCCTTAGCTCCTGAAAA | |
| | TCTCGATAACTCAAAAAATACGCCCGGTAGTGATCTTATTTCATTATGGTG | |
| 77 | CTGAGCGTCAGACCCCGTAGAAAAGATCAAAGGATCTTCTTGAGATCCTTTTTTT | intermediate vector |
| | CTGCGCGTAATCTGCTGCTTGCAAACAAAAAAACCACCGCTACCAGCGGTGGTTT | 2 (psl040-2nd^{t}- |
| | GTTTGCCGGATCAAGAGCTACCAACTCTTTTTCCGAAGGTAACTGGCTTCAGCAG | acceptor vector for |
| | AGCGCAGATACCAAATACTGTTCTTCTAGTGTAGCCGTAGTTAGGCCACCACTTC | up to 6 grnas |
| | AAGAACTCTGTAGCACCGCCTACATACCTCGCTCTGCTAATCCTGTTACCAGTGGC | |
| | TGCTGCCAGTGGCGATAAGTCGTGTCTTACCGGGTTGGACTCAAGACGATAGTT | |
| | ACCGGATAAGGCGCAGCGGTCGGGCTGAACGGGGGETTCGTGCACACAGCCCA | |
| | GCTTGGAGCGAACGACCTACACCGAACTGAGATACCTACAGCGTGAGCTATGAG | |
| | AAAGCGCCACGCTTCCCGAAGGGAGAAAGGCGGACAGGTATCCGGTAAGCGGC | |
| | AGGGTCGGAACAGGAGAGCGCACGAGGGAGCTTCCAGGGGGAAACGCCTGGT | |
| | ATCTTTATAGTCCTGTCGGGTTTCGCCACCTCTGACTTGAGCGTCGATTTTTGTGA | |
| | TGCTCGTCAGGGGGGGCCAGCAACGCGGCCTTTTTACGGTTCCTGGCCTTTTGCT | |
| | GGCCTTTTGCTCACATGTTCTTTCCTGCGTTATCCCCTGATTCTGTGGATAACCGT | |
| | AGGGTCTCATGCCCTGCCGAGACGGAAAGTGAAACGTGATTTCATGCGTCATTTT | |
| | GAACATTTTGTAAATCTTATTTAATAATGTGTGCGGCAATTCACATTTAATTTATG | |
| | AATGTTTTCTTAACATCGCGGCAACTCAAGAAACGGCAGGTTCGGATCTTAGCTA | |
| | CTAGAGAAAGAGGAGAAATACTAGATGCGTAAAGGCGAAGAGCTGTTCACTGG | |
| | TGTCGTCCCTATTCTGGTGGAACTGGATGGTGATGTCAACGGTCATAAGTTTTCC | |
| | GTGCGTGGCGAGGGTGAAGGTGACGCAACTAATGGTAAACTGACGCTGAAGTT | |
| | CATCTGTACTACTGGTAAACTGCCGGTTCCTTGGCCGACTCTGGTAACGACGCTG | |
| | ACTTATGGTGTTCAGTGCTTTGCTCGTTATCCGGACCATATGAAGCAGCATGACTT | |
| | CTTCAAGTCCGCCATGCCGGAAGGCTATGTGCAGGAACGCACGATTTCCTTTAAG | |
| | GATGACGGCACGTACAAAACGCGTGCGGAAGTGAAATTTGAAGGCGATACCCTG | |
| | GTAAACCGCATTGAGCTGAAAGGCATTGACTTTAAAGAGGACGGCAATATCCTG | |
| | GGCCATAAGCTGGAATACAATTTTAACAGCCACAATGTTTACATCACCGCCGATA | |
| | AACAAAAAAATGGCATTAAAGCGAATTTTAAAATTCGCCACAACGTGGAGGATG | |
| | GCAGCGTGCAGCTGGCTGATCACTACCAGCAAAACACTCCAATCGGTGATGGTC | |
| | CTGTTCTGCTGCCAGACAATCACTATCTGAGCACGCAAAGCGTTCTGTCTAAAGA | |
| | TCCGAACGAGAAACGCGATCATATGGTTCTGCTGGAGTTCGTAACCGCAGCGGG | |
| | CATCACGCATGGTATGGATGAACTGTACAAATGACCAGGCATCAAATAAAACGA | |
| | AAGGCTCAGTCGAAAGACTGGGCCTTTCGTTTTATCTGTTGTTTGTCGGTGAACG | |
| | CTCTCTACTAGAGTCACACTGGCTCACCTTCGGGTGGGCCTTTCTGCGTTTATACG | |
| | TCTCTATCCCTAATGAGACCAGACCAATAAAAAACGCCCGGCGGCAACCGAGCG | |
| | TTCTGAACAAATCCAGATGGAGTTCTGAGGTCATTACTGGATCTATCAACAGGAG | |
| | TCCAAGCGAGCTCGATATCAAATTACGCCCCGCCCTGCCACTCATCGCAGTACTG | |
| | TTGTAATTCATTAAGCATTCTGCCGACATGGAAGCCATCACAAACGGCATGATGA | |
| | ACCTGAATCGCCAGCGGCATCAGCACCTTGTCGCCTTGCGTATAATATTTGCCCAT | |
| | GGTGAAAACGGGGGCGAAGAAGTTGTCCATATTGGCCACGTTTAAATCAAAACT | |
| | GGTGAAACTCACCCAGGGATTGGCTGAAACGAAAAACATATTCTCAATAAACCCT | |
| | TTAGGGAAATAGGCCAGGTTTTCACCGTAACACGCCACATCTTGCGAATATATGT | |
| | GTAGAAACTGCCGGAAATCGTCGTGGTATTCACTCCAGAGCGATGAAAACGTTT | |
| | CAGTTTGCTCATGGAAAACGGTGTAACAAGGGTGAACACTATCCCATATCACCAG | |
| | CTCACCGTCTTTCATTGCCATACGAAATTCCGGATGAGCATTCATCAGGCGGGCA | |
| | AGAATGTGAATAAAGGCCGGATAAAACTTGTGCTTATTTTTCTTTACGGTCTTTAA | |
| | AAAGGCCGTAATATCCAGCTGAACGGTCTGGTTATAGGTACATTGAGCAACTGA | |
| | CTGAAATGCCTCAAAATGTTCTTTACGATGCCATTGGGATATATCAACGGTGGTA | |
| | TATCCAGTGATTTTTTTCTCCATTTTAGCTTCCTTAGCTCCTGAAAATCTCGATAAC | |
| | TCAAAAAATACGCCCGGTAGTGATCTTATTTCATTATGGTGAAAGTTGGAACCTC | |
| | TTACGTGCCCGATCAATCATGACCAAAATCCCTTAACGTGAGTTTTCGTTCCA | |
| 78 | CTCCAGAGCGATGAAAACGTTTCAGTTTGCTCATGGAAAACGGTGTAACAAGGG | Intermediate vector |
| | TGAACACTATCCCATATCACCAGCTCACCGTCTTTCATTGCCATACGAAATTCCGG | 3 (psl040-3rd- |
| | ATGAGCATTCATCAGGCGGGCAAGAATGTGAATAAAGGCCGGATAAAACTTGTG | acceptor vector for |
| | CTTATTTTTCTTTACGGTCTTTAAAAAGGCCGTAATATCCAGCTGAACGGTCTGGT | up to 6 grnas |
| | TATAGGTACATTGAGCAACTGACTGAAATGCCTCAAAATGTTCTTTACGATGCCA | |
| | TTGGGATATATCAACGGTGGTATATCCAGTGATTTTTTTCTCCATTTTAGCTTCCTT | |
| | AGCTCCTGAAAATCTCGATAACTCAAAAAATACGCCCGGTAGTGATCTTATTTCAT | |
| | TATGGTGAAAGTTGGAACCTCTTACGTGCCCGATCAATCATGACCAAAATCCCTT | |
| | AACGTGAGTTTTCGTTCCACTGAGCGTCAGACCCCGTAGAAAAGATCAAAGGAT | |
| | CTTCTTGAGATCCTTTTTTTCTGCGCGTAATCTGCTGCTTGCAAACAAAAAAACCA | |
| | CCGCTACCAGCGGTGGTTTGTTTGCCGGATCAAGAGCTACCAACTCTTTTTCCGA | |
| | AGGTAACTGGCTTCAGCAGAGCGCAGATACCAAATACTGTTCTTCTAGTGTAGCC | |
| | GTAGTTAGGCCACCACTTCAAGAACTCTGTAGCACCGCCTACATACCTCGCTCTG | |
| | CTAATCCTGTTACCAGTGGCTGCTGCCAGTGGCGATAAGTCGTGTCTTACCGGGT | |
| | TGGACTCAAGACGATAGTTACCGGATAAGGCGCAGCGGTCGGGCTGAACGGGG | |
| | GGTTCGTGCACACAGCCCAGCTTGGAGCGAACGACCTACACCGAACTGAGATAC | |
| | CTACAGCGTGAGCTATGAGAAAGCGCCACGCTTCCCGAAGGGAGAAAGGCGGA | |
| | CAGGTATCCGGTAAGCGGCAGGGTCGGAACAGGAGAGCGCACGAGGGAGCTTC | |
| | CAGGGGGAAACGCCTGGTATCTTTATAGTCCTGTCGGGTTTCGCCACCTCTGACT | |
| | TGAGCGTCGATTTTTGTGATGCTCGTCAGGGGGGGCCAGCAACGCGGCCTTTTTA | |
| | CGGTTCCTGGCCTTTTGCTGGCCTTTTGCTCACATGTTCTTTCCTGCGTTATCCCCT | |
| | GATTCTGTGGATAACCGTAGGGTCTCACTAACTGCCGAGACGGAAAGTGAAACG | |
| | TGATTTCATGCGTCATTTTGAACATTTTGTAAATCTTATTTAATAATGTGTGCGGC | |
| | AATTCACATTTAATTTATGAATGTTTTCTTAACATCGCGGCAACTCAAGAAACGGC | |
| | AGGTTCGGATCTTAGCTACTAGAGAAAGAGGAGAAATACTAGATGCGTAAAGGC | |
| | GAAGAGCTGTTCACTGGTGTCGTCCCTATTCTGETGGAACTGGATGGTGATGTCA | |
| | ACGGTCATAAGTTTTCCGTGCGTGGCGAGGGTGAAGGTGACGCAACTAATGGTA | |
| | AACTGACGCTGAAGTTCATCTGTACTACTGGTAAACTGCCGGTTCCTTGGCCGAC | |
| | TCTGGTAACGACGCTGACTTATGGTGTTCAGTGCTTTGCTCGTTATCCGGACCATA | |
| | TGAAGCAGCATGACTTCTTCAAGTCCGCCATGCCGGAAGGCTATGTGCAGGAAC | |
| | GCACGATTTCCTTTAAGGATGACGGCACGTACAAAACGCGTGCGGAAGTGAAAT | |
| | TTGAAGGCGATACCCTGGTAAACCGCATTGAGCTGAAAGGCATTGACTTTAAAG | |
| | AGGACGGCAATATCCTGGGCCATAAGCTGGAATACAATTTTAACAGCCACAATGT | |
| | TTACATCACCGCCGATAAACAAAAAAATGGCATTAAAGCGAATTTTAAAATTCGC | |
| | CACAACGTGGAGGATGGCAGCGTGCAGCTGGCTGATCACTACCAGCAAAACACT | |
| | CCAATCGGTGATGGTCCTGTTCTGCTGCCAGACAATCACTATCTGAGCACGCAAA | |
| | GCGTTCTGTCTAAAGATCCGAACGAGAAACGCGATCATATGGTTCTGCTGGAGTT | |
| | CGTAACCGCAGCGGGCATCACGCATGGTATGGATGAACTGTACAAATGACCAGG | |
| | CATCAAATAAAACGAAAGGCTCAGTCGAAAGACTGGGCCTTTCGTTTTATCTGTT | |
| | GTTTGTCGGTGAACGCTCTCTACTAGAGTCACACTGGCTCACCTTCGGGTGGGCC | |
| | TTTCTGCGTTTATACGTCTCTATCCACCATGAGACCAGACCAATAAAAAACGCCCG | |
| | GCGGCAACCGAGCGTTCTGAACAAATCCAGATGGAGTTCTGAGGTCATTACTGG | |
| | ATCTATCAACAGGAGTCCAAGCGAGCTCGATATCAAATTACGCCCCGCCCTGCCA | |
| | CTCATCGCAGTACTGTTGTAATTCATTAAGCATTCTGCCGACATGGAAGCCATCAC | |
| | AAACGGCATGATGAACCTGAATCGCCAGCGGCATCAGCACCTTGTCGCCTTGCGT | |
| | ATAATATTTGCCCATGGTGAAAACGGGGGCGAAGAAGTTGTCCATATTGGCCAC | |
| | GTTTAAATCAAAACTGGTGAAACTCACCCAGGGATTGGCTGAAACGAAAAACAT | |
| | ATTCTCAATAAACCCTTTAGGGAAATAGGCCAGGTTTTCACCGTAACACGCCACA | |
| | TCTTGCGAATATATGTGTAGAAACTGCCGGAAATCGTCGTGGTATTCA | |
| 79 | GCACGAGGGAGCTTCCAGGGGGAAACGCCTGGTATCTTTATAGTCCTGTCGGGT | Intermediate vector |
| | TTCGCCACCTCTGACTTGAGCGTCGATTTTTGTGATGCTCGTCAGGGGGGGCCAG | 4 (psl040-4th- |
| | CAACGCGGCCTTTTTACGGTTCCTGGCCTTTTGCTGGCCTTTTGCTCACATGTTCTT | acceptor vector for |
| | TCCTGCGTTATCCCCTGATTCTGTGGATAACCGTAGGGTCTCAACCACTGCCGAG | up to 6 grnas |
| | ACGGAAAGTGAAACGTGATTTCATGCGTCATTTTGAACATTTTGTAAATCTTATTT | |
| | AATAATGTGTGCGGCAATTCACATTTAATTTATGAATGTTTTCTTAACATCGCGGC | |
| | AACTCAAGAAACGGCAGGTTCGGATCTTAGCTACTAGAGAAAGAGGAGAAATAC | |
| | TAGATGCGTAAAGGCGAAGAGCTGTTCACTGGTGTCGTCCCTATTCTGGTGGAA | |
| | CTGGATGGTGATGTCAACGGTCATAAGTTTTCCGTGCGTGGCGAGGGTGAAGGT | |
| | GACGCAACTAATGGTAAACTGACGCTGAAGTTCATCTGTACTACTGGTAAACTGC | |
| | CGGTTCCTTGGCCGACTCTGGTAACGACGCTGACTTATGGTGTTCAGTGCTTTGC | |
| | TCGTTATCCGGACCATATGAAGCAGCATGACTTCTTCAAGTCCGCCATGCCGGAA | |
| | GGCTATGTGCAGGAACGCACGATTTCCTTTAAGGATGACGGCACGTACAAAACG | |
| | CGTGCGGAAGTGAAATTTGAAGGCGATACCCTGGTAAACCGCATTGAGCTGAAA | |
| | GGCATTGACTTTAAAGAGGACGGCAATATCCTGGGCCATAAGCTGGAATACAAT | |
| | TTTAACAGCCACAATGTTTACATCACCGCCGATAAACAAAAAAATGGCATTAAAG | |
| | CGAATTTTAAAATTCGCCACAACGTGGAGGATGGCAGCGTGCAGCTGGCTGATC | |
| | ACTACCAGCAAAACACTCCAATCGGTGATGGTCCTGTTCTGCTGCCAGACAATCA | |
| | CTATCTGAGCACGCAAAGCGTTCTGTCTAAAGATCCGAACGAGAAACGCGATCAT | |
| | ATGGTTCTGCTGGAGTTCGTAACCGCAGCGGGCATCACGCATGGTATGGATGAA | |
| | CTGTACAAATGACCAGGCATCAAATAAAACGAAAGGCTCAGTCGAAAGACTGGG | |
| | CCTTTCGTTTTATCTGTTGTTTGTCGGTGAACGCTCTCTACTAGAGTCACACTGGC | |
| | TCACCTTCGGGTGGGCCTTTCTGCGTTTATACGTCTCTATCCATCCTGAGACCAGA | |
| | CCAATAAAAAACGCCCGGCGGCAACCGAGCGTTCTGAACAAATCCAGATGGAGT | |
| | TCTGAGGTCATTACTGGATCTATCAACAGGAGTCCAAGCGAGCTCGATATCAAAT | |
| | TACGCCCCGCCCTGCCACTCATCGCAGTACTGTTGTAATTCATTAAGCATTCTGCC | |
| | GACATGGAAGCCATCACAAACGGCATGATGAACCTGAATCGCCAGCGGCATCAG | |
| | CACCTTGTCGCCTTGCGTATAATATTTGCCCATGGTGAAAACGGGGGCGAAGAA | |
| | GTTGTCCATATTGGCCACGTTTAAATCAAAACTGGTGAAACTCACCCAGGGATTG | |
| | GCTGAAACGAAAAACATATTCTCAATAAACCCTTTAGGGAAATAGGCCAGGTTTT | |
| | CACCGTAACACGCCACATCTTGCGAATATATGTGTAGAAACTGCCGGAAATCGTC | |
| | GTGGTATTCACTCCAGAGCGATGAAAACGTTTCAGTTTGCTCATGGAAAACGGTG | |
| | TAACAAGGGTGAACACTATCCCATATCACCAGCTCACCGTCTTTCATTGCCATACG | |
| | AAATTCCGGATGAGCATTCATCAGGCGGGCAAGAATGTGAATAAAGGCCGGATA | |
| | AAACTTGTGCTTATTTTTCTTTACGGTCTTTAAAAAGGCCGTAATATCCAGCTGAA | |
| | CGETCTGGTTATAGGTACATTGAGCAACTGACTGAAATGCCTCAAAATGTTCTTT | |
| | ACGATGCCATTGGGATATATCAACGGTGGTATATCCAGTGATTTTTTTCTCCATTT | |
| | TAGCTTCCTTAGCTCCTGAAAATCTCGATAACTCAAAAAATACGCCCGGTAGTGA | |
| | TCTTATTTCATTATGGTGAAAGTTGGAACCTCTTACGTGCCCGATCAATCATGACC | |
| | AAAATCCCTTAACGTGAGTTTTCGTTCCACTGAGCGTCAGACCCCGTAGAAAAGA | |
| | TCAAAGGATCTTCTTGAGATCCTTTTTTTCTGCGCGTAATCTGCTGCTTGCAAACA | |
| | AAAAAACCACCGCTACCAGCGGTGGTTTGTTTGCCGGATCAAGAGCTACCAACTC | |
| | TTTTTCCGAAGGTAACTGGCTTCAGCAGAGCGCAGATACCAAATACTGTTCTTCT | |
| | AGTGTAGCCGTAGTTAGGCCACCACTTCAAGAACTCTGTAGCACCGCCTACATAC | |
| | CTCGCTCTGCTAATCCTGTTACCAGTGGCTGCTGCCAGTGGCGATAAGTCGTGTC | |
| | TrACCGGETTGGACTCAAGACGATAGTTACCGGATAAGGCGCAGCGGTCGGGCT | |
| | GAACGGGGGGTTCGTGCACACAGCCCAGCTTGGAGCGAACGACCTACACCGAAC | |
| | TGAGATACCTACAGCGTGAGCTATGAGAAAGCGCCACGCTTCCCGAAGGGAGAA | |
| | AGGCGGACAGGTATCCGGTAAGCGGCAGGGTCGGAACAGGAGAGC | |
| 81 | | |

### FIGURE LEGENDS

FIG. 1: Schematic showing exemplary method for producing an RNA mediated gene regulating nucleic acid construct that comprises at least two sequences that are transcribed into nucleic acid polymers that each separately direct RNA mediated gene regulation, wherein the at least two nucleic acid sequences are transcribed into a single transcript from a single promoter

FIG. 2: CHORDS assembly and efficiency.

(A) Schematic overview of one particular embodiment of the method for the construction of gRNA arrays. A Guide-Generating Vector is first used to add the gRNA targeting sequence of interest, via a designed forward primer overhang and a fixed, phosphorylated reverse primer. The generated, linear PCR fragment with the added gRNA is then annealed. The resulting, circularized vector is then amplified in a second round of PCR, in which both a forward and reverse primer are used to add designed BsmBI overhangs. The resulting PCR fragments can then be inserted into a Destination Vector containing a promoter, 3′ Csy4 site and terminator via Golden Gate assembly. Primers are indicated by arrows, with slanted lines indicating primer overhangs. (B) BsmBI recognition site and 4 bp overhangs used in this study. Twelve different 4 bp overhangs were validated for use with CHORDS. Shaded brown rectangle indicates the Type IIs BsmBI restriction enzyme, which recognizes the sequence 5′-CGTCTC-3′ and generates an adjacent 4 bp overhang. (C) (Left) Assembly efficiency for the construction of gRNA arrays with CHORDS. White colonies were counted and compared to the total E. coli colonies (white indicating GFP-negative) after CHORDS assembly (n=8 transformed and streaked plates, 50 μl cells, for each condition). Error bars represent the standard deviation in white/total counts between the replicates. (Right) Restriction digests with BsaI were used to validate insert size within the Destination Vectors (n=16 colonies each condition).

FIG. 3: Multiplexing of gRNAs for combinatorial transcriptional repression in S. cerevisiae.

(A) Spatial positions of the gRNAs tested and containing 20 nt sequences complementary to the ScALD6, ScHHF1 or ScTEF1 and adjacent to a PAM sequence 5′-NGG-3. gRNAs were targeted between −300 bp upstream and +1 bp downstream of the start codon.

Numbers in the gray boxes correspond to the results plotted in panel (B) for each of the three fluorescent reporters. (B) Relative repression of fluorescence for each gRNA tested with n=4 biological replicates each condition. (C) Relative repression of fluorescence by combinatorial, multiplexed expression of gRNA arrays. Each gRNA array (from 3 through 12) has an additional three gRNAs, one targeting each of the fluorescent reporters in our system and validated from (B). WT, wildtype BY4741 yeast; -gRNAs, no gRNA expressed. RFU, relative fluorescence units. All values plotted are mean averages from n=8 samples (3, 6, 9, 12 gRNA arrays) or n=4 (WT, -gRNA, Blank 3-part) and error bars represent one standard deviation from the mean. Asterisks denote two-tail p-value as determined by two-sample t-test, with *p≤0.05, **p≤0.01, and ***p≤0.0001.

FIG. 4: Experimental protocol schematic for CHORDS Assembly. Arrows indicate the steps through the protocol over a two-day period.

FIG. 5: Schematic

FIG. 6: Up to 12 gRNAs are Expressed in S. cerevisiae and Enable Highly Multiplexed Regulation of Gene Expression.

Combinatorial repression of three targets simultaneously via highly multiplexed gRNA expression. mVenus (left), mTagBFP (center) and mRuby2 fluorescence (right) in BY4741 expressing green, blue and red fluorescent proteins, dCas9 and Csy4. This strain was transformed with either a blank integration vector, one blank gRNA, three blank gRNAs, or 3, 6, 9 or 12-guide assemblies constructed by CHORDS and fluorescence measured via three-channel flow cytometry. *, p<0.05; **, p<0.005; ‡, p<0.001; n.s., not significant. Statistics assessed by student's t-test for each condition compared to the strain indicated by the connecting black line. BY4741 (WT), URA3 blank integration, one blank guide, 3 blank guides are the mean of n=4 samples ±SD, while the 3, 6, 9 and 12-guide assemblies are the mean of n=8 samples ±SD. RFU, relative fluorescence units.

FIG. 7: Frequency of cleavage of restriction sites in some common nucleic acid molecules

FIG. 8: Exemplary method according to the invention, wherein at least two different nucleic acid arrays are cloned into intermediate vectors and are then subsequently cloned (either directly by digestion of the intermediate vector, or indirectly by amplification of the nucleic acid array) into a single destination or expression vector.

We illustrate exemplary embodiments of the present invention in the following non-limiting examples.

### EXAMPLES

### Example 1

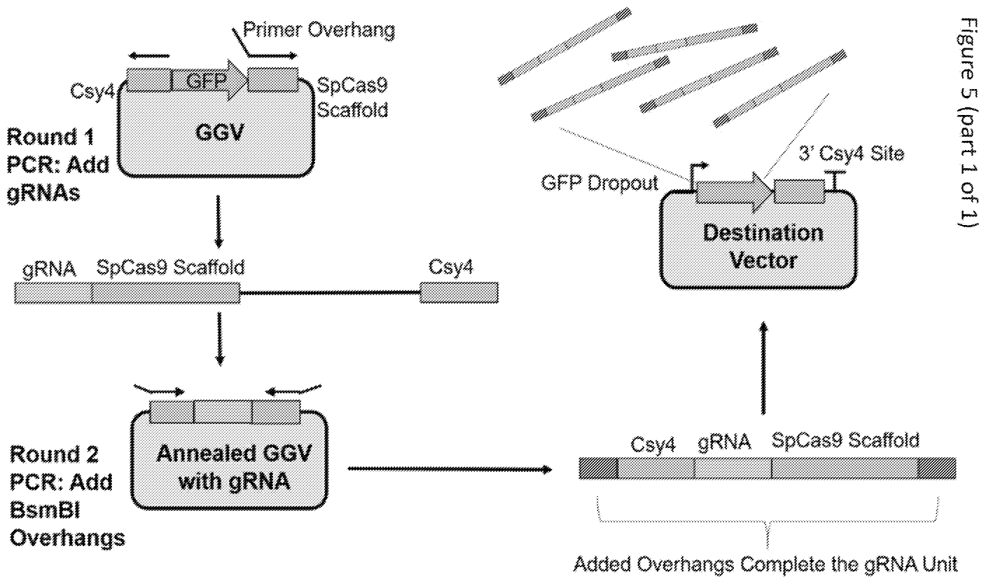

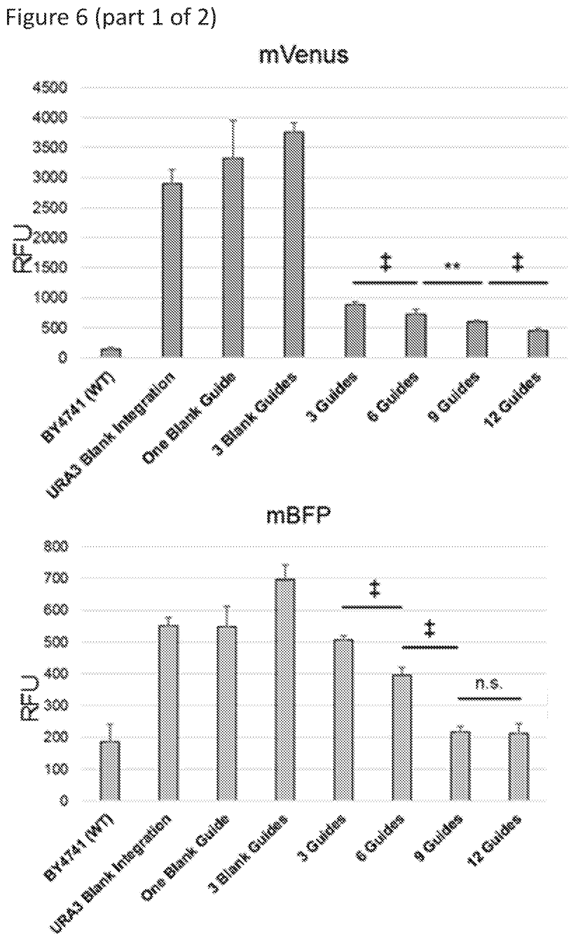

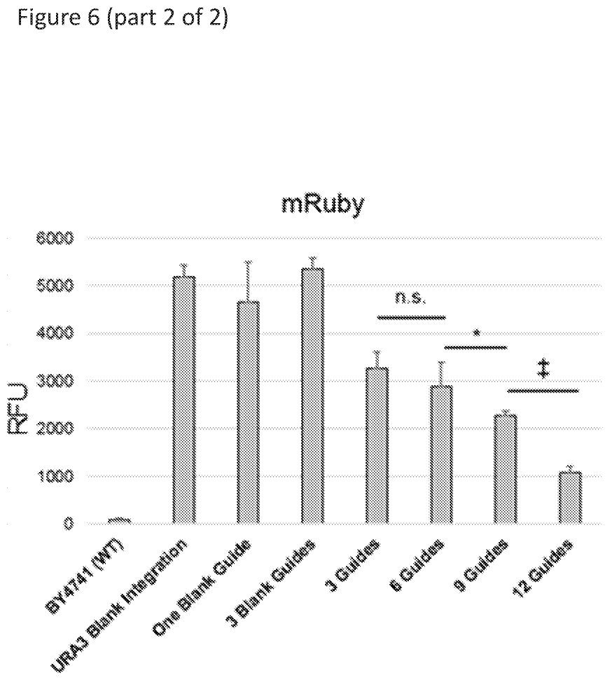

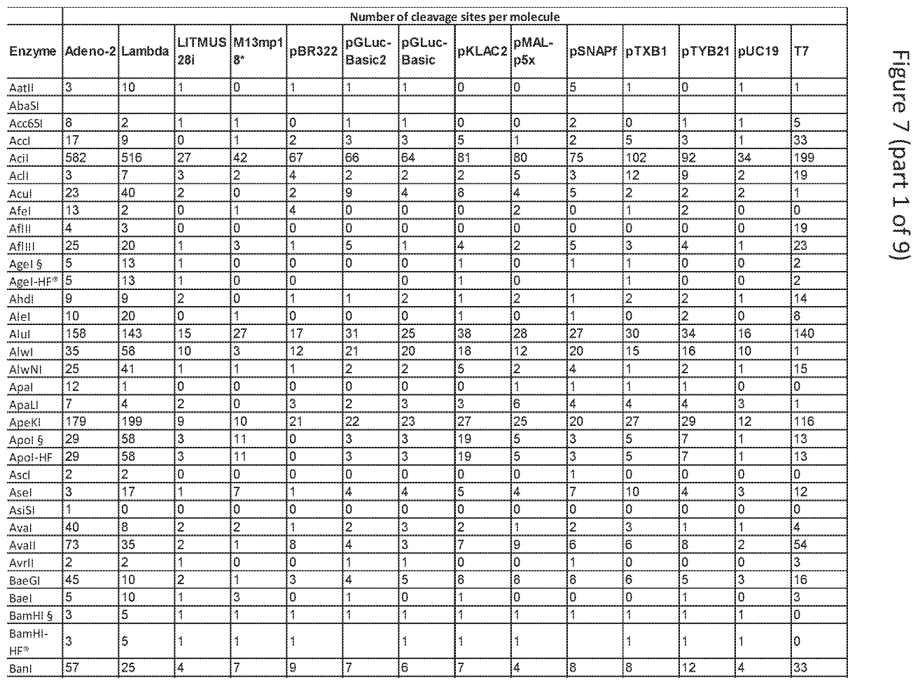

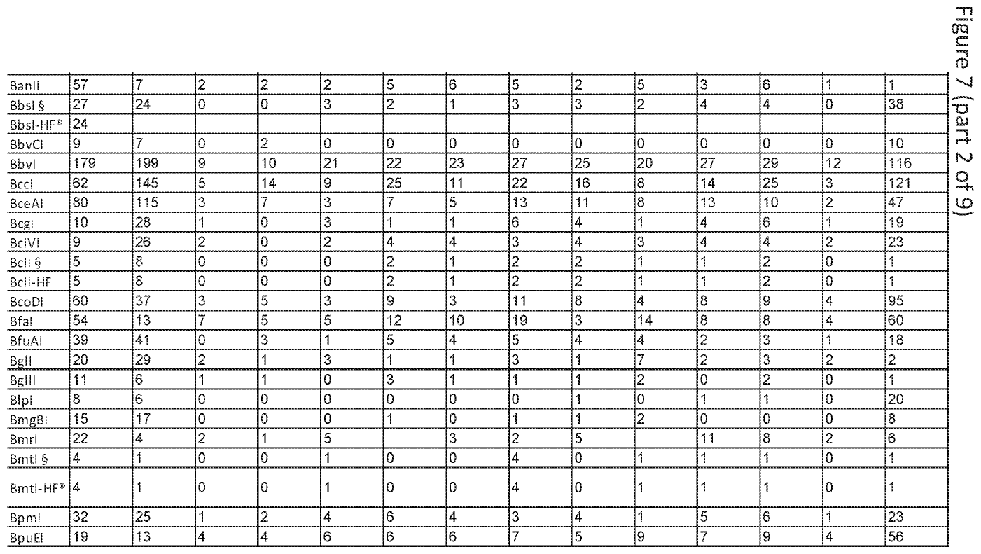

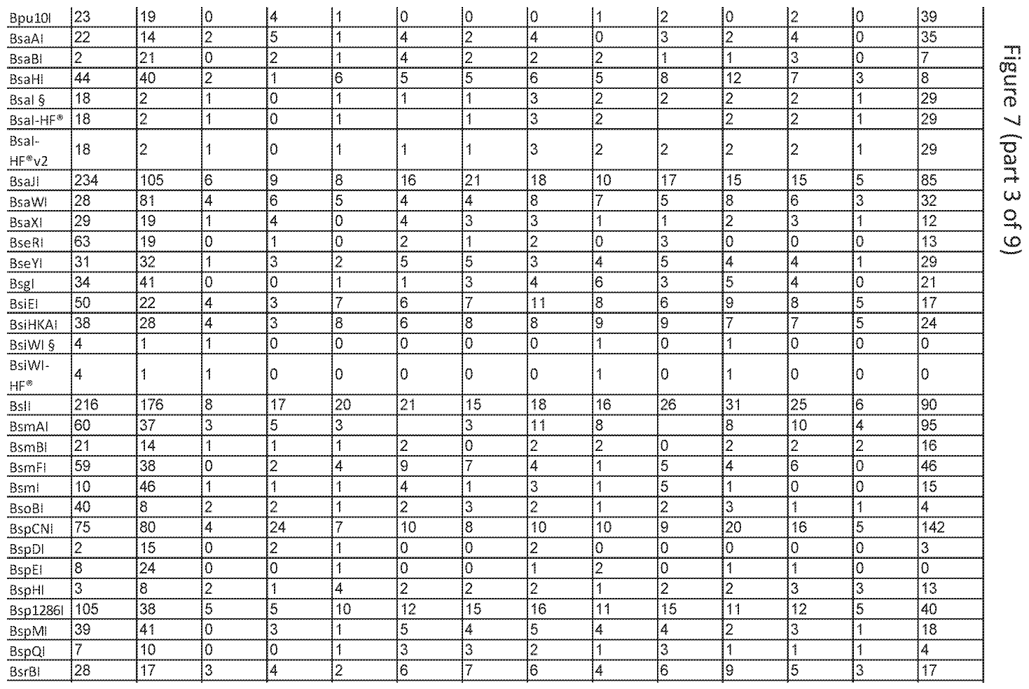

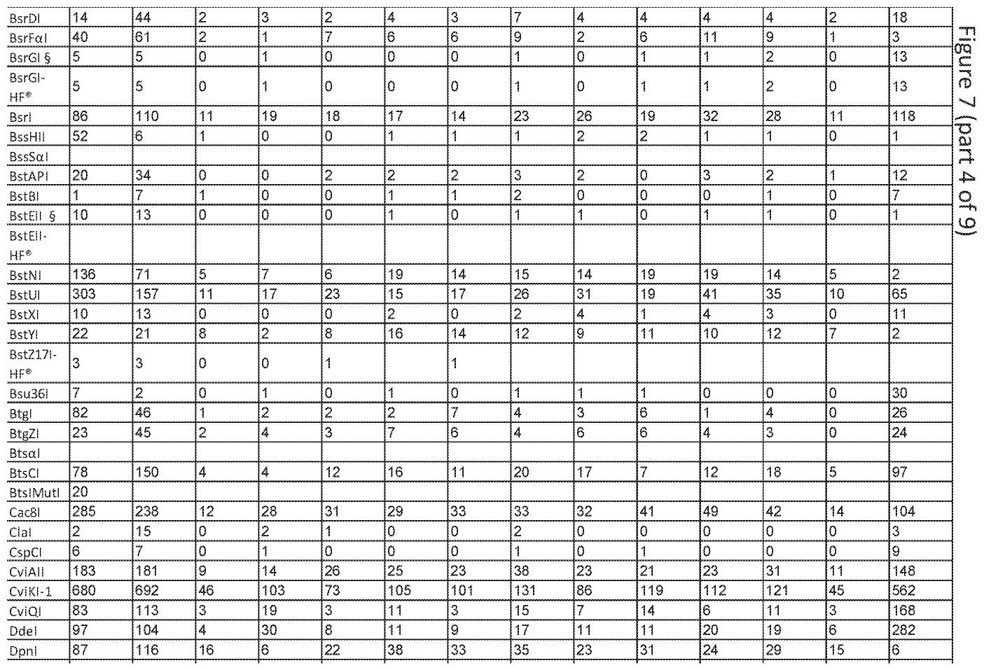

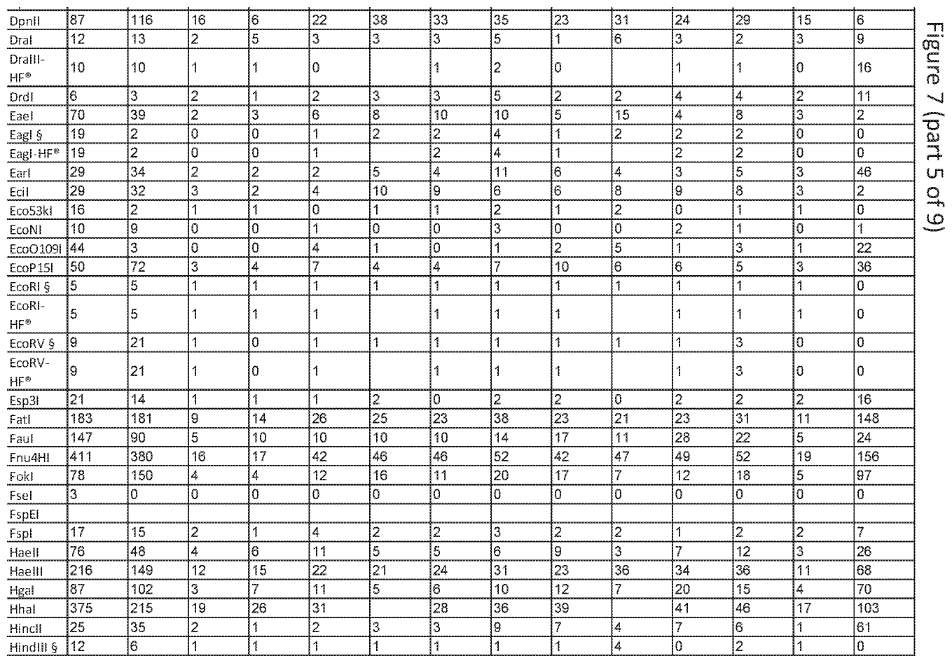

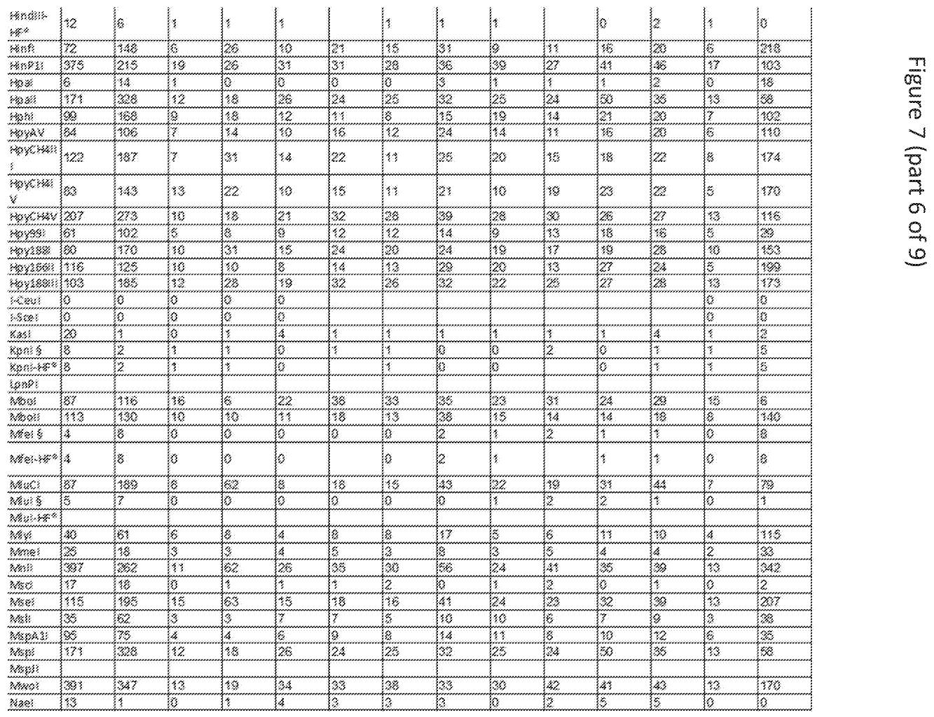

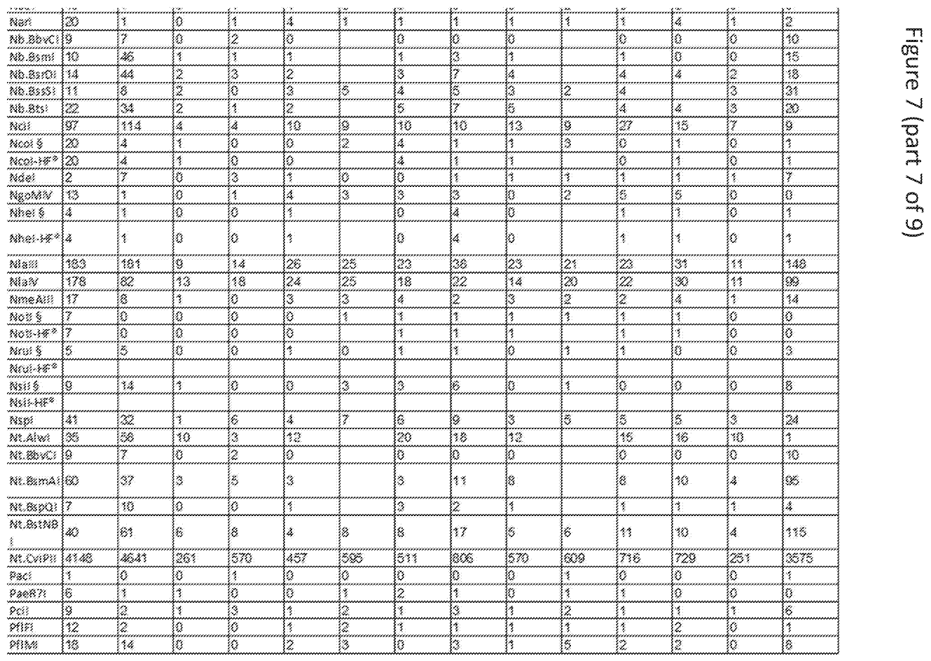

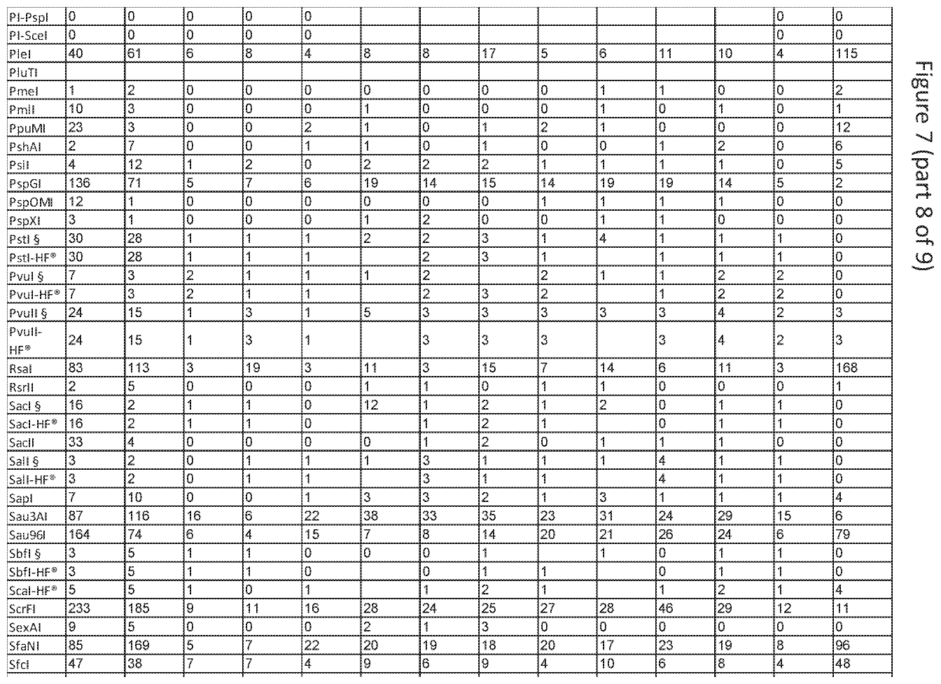

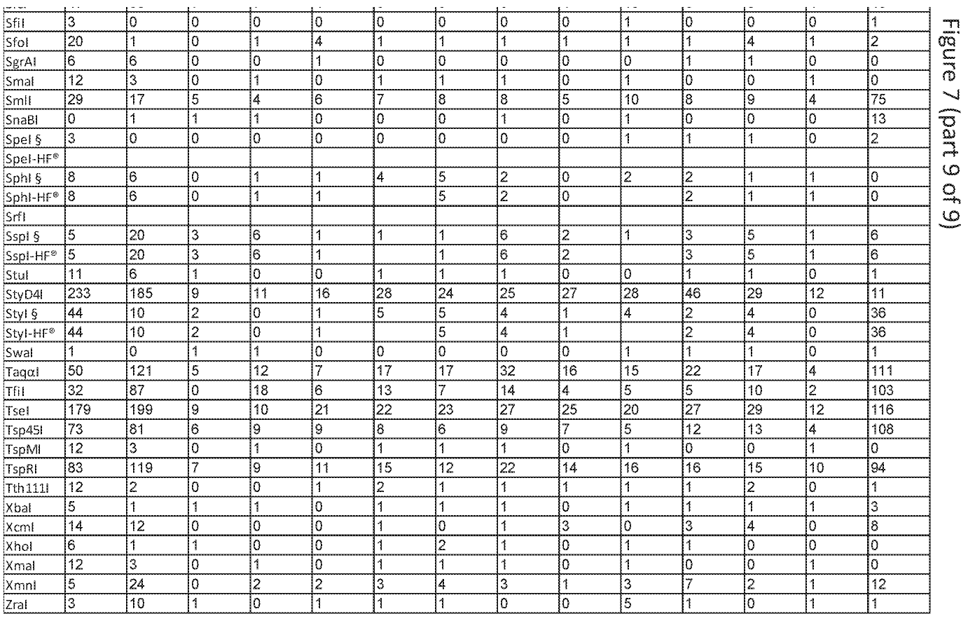

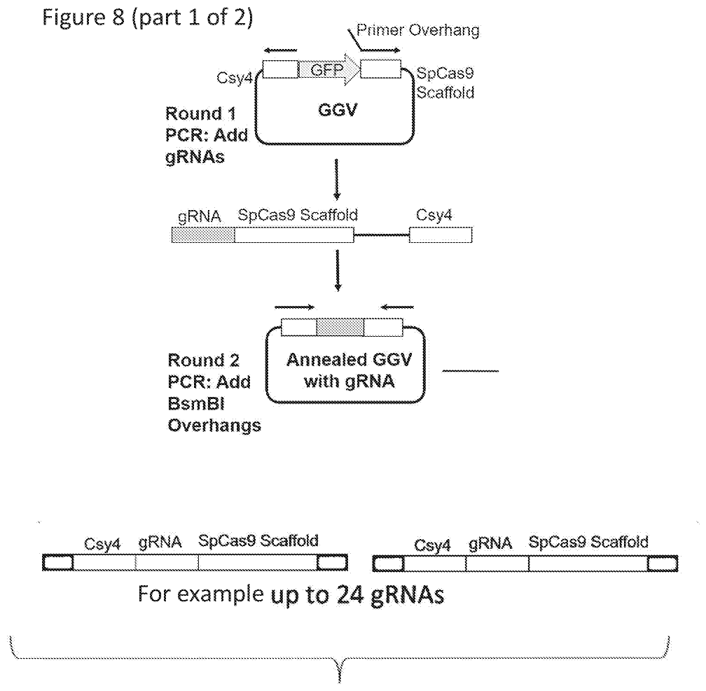

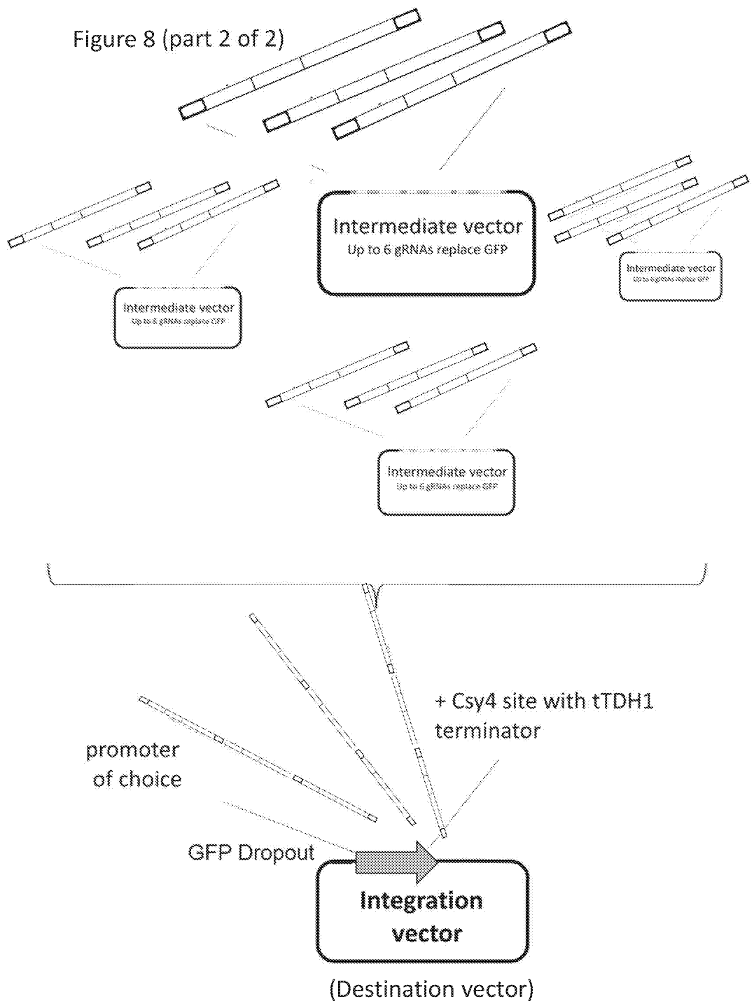

The efficiency of CHORDS assembly was tested for the construction of highly repetitive DNA sequences. As a proof-of-concept, a series of gRNA arrays were built containing an increasing number of gRNAs (3, 6, 9 or 12) within a single transcriptional unit (FIG. 2a). Components compatible with the YTK were created due to the expansive use of this toolkit in synthetic biology research and the total absence of existing multiplexing gRNA systems for yeasts, the most industrially-relevant organism.

Briefly, PCR with a high-fidelity Phusion polymerase was used to add the gRNA sequence of interest to a Guide-Generating Vector, which consists of a 20 nt Csy4 recognition site followed by a superfolder GFP gene and a 3′ Cas9 scaffold. The forward primer adds the gRNA targeting sequence via primer overhangs, while a phosphorylated reverse primer completes replication of the PCR fragment and results in dropout of the sfGFP, which facilitates E. coli colony screening. The resulting, linear PCR fragment is annealed, and a second round of PCR performed to add BsmBI restriction sites with pre-defined 4 bp overhangs (FIG. 2b). The resulting PCR fragments can then be inserted into a Destination Vector, which consists of a promoter, sfGFP gene, 3′ Csy4 recognition site and terminator, via Golden Gate assembly. New destination vectors can be made in one day via Gibson Assembly with current promoters and terminators in the standard YTK. The destination vectors also contain designed BsaI cut sites for straightforward diagnostic restriction digestion and designed XhoI/BglII sites on the 3′ end of the promoter and 5′ end of the terminator, respectively, to enable the swapping of constructed gRNA arrays between different destination vectors.

After Golden Gate assembly, TurboComp E. coli were chemically transformed and plated on LB containing chloramphenicol. Screening of these colonies for expression of GFP under UV light was used to assess the ratio of colonies containing some form of our genetic construct (FIG. 2c, left). For construction of gRNA arrays with 3, 6 and 9 gRNAs, >98% of E. coli colonies were GFP negative. For E. coli transformed with the 12 gRNA array, >96% of E. coli colonies were GFP negative.

To validate the true assembly efficiency of CHORDS, however, insert length was screened for within the destination vector via diagnostic restriction digest with BsaI and then sequence-verified putative colonies by Sanger sequencing (see Supplemental Information). As expected, restriction digests of the arrays indicated a decrease in assembly efficiency with higher orders of gRNAs. A construction efficiency >40% was observed on gRNA arrays up to 9 gRNAs, with a subsequent drop-off in efficiency for higher orders of gRNAs (FIG. 2c). All colonies with expected restriction digest band patterns sent for sequencing were sequence-verified without any observed mutations.

To demonstrate the utility of CHORDS in an industrially-relevant model organism, the multiplexing capabilities of gRNAs expressed from a single promoter in S. cerevisiae was tested. It was hypothesized that, due to elevated rates of homologous recombination at genomic regions containing highly repetitive DNA sequences, only a few gRNAs could be expressed from a single promoter in S. cerevisiae. An experiment was designed to test the multiplexing limits of gRNAs in yeast which did not rely on quantitative PCR, as the high similarity between the gRNAs could confound quantitation of our transcript counts. Instead, a flow cytometry experiment was designed in which a series of fluorescent reporters (green, blue and red) are transcriptionally repressed by increasing numbers of gRNAs.

Golden Gate and the YTK was first used to engineer S. cerevisiae strain BY4741 to express three fluorescent reporters, ScTEF1-mTagBFP2, ScHHF1-mRuby2 and ScALD6-Venus, which were genome-integrated at the HO-site. This yeast strain was also transformed with a LEU2-integrated vector that expresses dCas9 with nuclear localization signals on the 5′ and 3′ ends, driven by the ScPGK1 promoter, and a Csy4 enzyme with a 5′ nuclear localization signal under control of the ScHHF2 promoter (BY4741^{-gRNAs}). Before constructing large arrays of gRNAs, the repression efficiency of different gRNAs was validated for each of the fluorescent reporters individually. BY4741^{-gRNAs} were transformed with single gRNAs (integrated at the URA3 locus) driven by the Pol III tRNA Phe promoter with a 5′ HDV ribozyme. Each gRNA targeted one of the three different promoters—TEF1, HHF1 and ALD6—and changes in fluorescence of each reporter following integration of the gRNA were assessed by flow cytometry (FIG. 3a). Each gRNA resulted in varied repression efficiencies and functioned orthogonally to one another (i.e. they did not repress other fluorescent reporters) (FIG. 3b). Using these results, we selected four gRNAs targeting each promoter based on two criteria: 1) Weak repression of fluorescent output (which was hypothesized to enable visualization of combinatorial effects when multiplexing) and 2) Distributed spatial positionings within the promoter region, which was hypothesized to enhance the likelihood of observing gRNA combinatorial effects for transcriptional repression. For mVenus repression, gRNAs #1, 4, 6, 8 targeting the ScALD6 promoter were used (in that order). For mRuby2 repression, gRNAs #2, 8, 6, 4 targeting the ScHHF1 promoter were used. For mTagBFP2 repression, gRNAs #1-4 targeting the ScTEF1 promoter were used.

Arrays of 3, 6, 9 or 12 gRNAs were built within a single transcriptional unit with CHORDS; as arrays increased in size, an additional gRNA was targeted to each fluorescent reporter. In the 12 gRNA array, for example, there are 4 gRNAs targeting the promoter upstream of each fluorescent reporter. Each gRNA is flanked by Csy4 recognition sites. Arrays were sequence-verified and then genome-integrated at the URA3 locus into BY474^{-gRNAs}. In the transformed yeast strains, a combinatorial, non-synergistic repression of fluorescence was observed in all three channels with increasing numbers of gRNAs targeted to each promoter (FIG. 3c). In all conditions except two, the expression of an additional gRNA resulted in a significant decrease in fluorescence of the respective reporter.

Since homologous recombination in bacteria and yeast is more active in regions containing repetitive DNA sequences,^{11,12} the stability of these repetitive gRNA arrays overtime was also assessed. Flow cytometry was performed every day for three days, with each yeast strain back-diluted 1:100 twice a day and grown for 12 hours between passages (FIG. 3d). Both flow cytometry data and colony PCR on yeast from day 1 and day 3 (5×1:100 dilutions) indicated sustained function and preservation of gRNA arrays overtime in vivo (FIG. 3e).

CHORDS offers a rapid and stable method by which large arrays of gRNAs can be constructed and utilized in vivo. This will facilitate applications in metabolic engineering prototyping and testing of genetic targets from computational predictions. This technology will enable the use of CRISPR for diverse applications in the multiplexed, transcriptional regulation of gene expression in this industrially-useful organism.

### Example 2

CHORDS Assembly

CHORDS assembly is a dual PCR, Type IIs Golden Gate method for constructing transcriptional units that contain repetitive DNA sequences flanked by short, variable DNA sequences. Dual PCR, in this case, refers to the two separate rounds of PCR which are performed in CHORDS assembly. After the two rounds of PCR, a Golden Gate reaction is performed to join all of the PCR fragments generated together in a one-pot reaction. FIG. 4 is a schematic/experimental guideline for performing CHORDS assembly. In the text that follows, the use of CHORDS for the assembly of highly repetitive gRNA arrays that are compatible with the Yeast Toolkit is described. However, it is strongly suspected that these primers and vectors could be modified for the assembly of other repetitive sequences, such as gRNAs flanked by introns or tRNAs, or to assemble repetitive Spinach aptamers.

The first step in CHORDS assembly to build gRNA arrays is to perform PCR on a ‘Guide-Generating Vector’ (template) with different combinations of primers. In round 1 PCR, the forward primer may have a 20 bp overhang on its 5′ end, which adds the gRNA target sequence of interest upon PCR amplification. A different forward primer must be ordered from an oligo manufacturer for every gRNA sequence to be constructed. In round 1 PCR, the reverse primer is fixed, meaning that it is the same primer for every reaction, and should be ordered from an oligo manufacturer with a phosphorylated 5′ end, which will facilitate ligation and re-circularization of these vectors in later steps.

Round 1 PCR Primers.

Primers for round 1 PCR, where N is the sequence of the gRNA from 5′ to 3′. 5′ Phos indicates that the 5′ end of the reverse primer should be ordered as a phosphorylated primer.

Forward Primer with Overhang -
[SEQ ID NO: 23]
NNNNNNNNNNNNNNNNNNNNgttttagagctagaaatagcaagttaaaata
ag
Reverse Primer -
[SEQ ID NO: 24]
5′ Phos-ctgcctatacggcagtgaac

Where N can be any length and any sequence, and denotes the gRNA targeting sequence.

During Round 1 PCR, the same template plasmid is used for all reactions. When constructing gRNA arrays flanked by Csy4 sites, a Guide-Generating Vector as described herein can be used.

Performing Round 1 PCR:

Components, concentrations and volumes to add to each PCR reaction mixture:

TABLE 2
PCR components for Round 1, which adds the desired
gRNA sequences.

| Component | Volume (μL) |
|---|---|
| Nuclease-free water | 31.5 |
| 5 × Phusion HF Buffer | 10 |
| dNTPs (10 mM) | 1 |
| Forward Primer (10 μM) | 2.5 |
| Reverse Primer (10 μM) | 2.5 |
| Guide-Generating Vector Template (10 ng/μL) | 0.5 |
| DMSO | 1.5 |
| Phusion Polymerase | 0.5 |
| Reaction volume | 50 |

Phusion Polymerase was used for CHORDS assembly due to its high-fidelity (see New England Biolabs product information: https://www.neb.com/faqs/2012/09/06/what-is-the-error-rate-of-phusion-reg-high-fldelity-dna-polymerase). In Phusion HF buffer, its reported fidelity is 4.4×10⁻⁷.

For each gRNA sequence to be constructed, a separate PCR reaction can be set up, with the only variation between reactions being the forward primer used.

PCR thermocycler conditions for Round 1 PCR:

TABLE 3
Thermocycler settings for Round 1 PCR.

| Step | Temp (° C.) | Time (s) |
|---|---|---|
| Initial Denaturation | 98 | 30 |
| 25-35 Cycles | 98 | 10 |
| | 61 | 30 |
| | 72 | 30 |
| Final Extension | 72 | 600 |
| Hold | 4 | |
| | PCR product | 1758 bp |
| | length | |

DpnI Digests:

After completing the Round 1 PCR, 0.3 μL of DpnI enzyme (purchased from New England Biolabs) is added to each PCR microtube. These samples are then incubated at 37° C. for 1 hour. DpnI cleaves methylated DNA—the Guide-Generating Vector in this case—and enhances isolation of the DNA fragments of interest in the next step by minimizing the likelihood that the template DNA is not isolated and used in the next round of PCR.

Gel Purify (1^{st} Time):

After DpnI digests, PCR tubes are removed from the thermocycler. The next step is to purify the DNA via gel electrophoresis and agarose gel extraction. This process is incredibly important to enhance the purity of the PCR fragments. Any contamination of the different PCR fragments in this step will mean that, in round 2 PCR (in which BsmBI restriction sites are added), multiple different gRNAs could be amplified with the same overhang primers. This would mean that there could be final constructs in which gRNAs are misplaced within the final array.

To minimize contamination, it is recommended that PCR fragments post-Dpn/digest be loaded in spatially separated wells (i.e. leave a well between samples) and to not overfill wells, as this could contaminate the other wells if DNA floats freely in the TAE buffer. For gel electrophoresis, it is sufficient to add, for example, ˜20 μL of the digested DNA mixture from the previous step to ˜3 μL of 6×DNA loading dye. This mixture is loaded into wells of a 0.8% agarose gel and gel electrophoresis is performed until total separation of DNA bands or for approximately 45 minutes at 100 volts. After gel electrophoresis, gel bands are excised. Zymoclean Gel DNA Recovery kit (Zymo Research) can be used, precisely followed manufacturer instructions.

T4 Ligation:

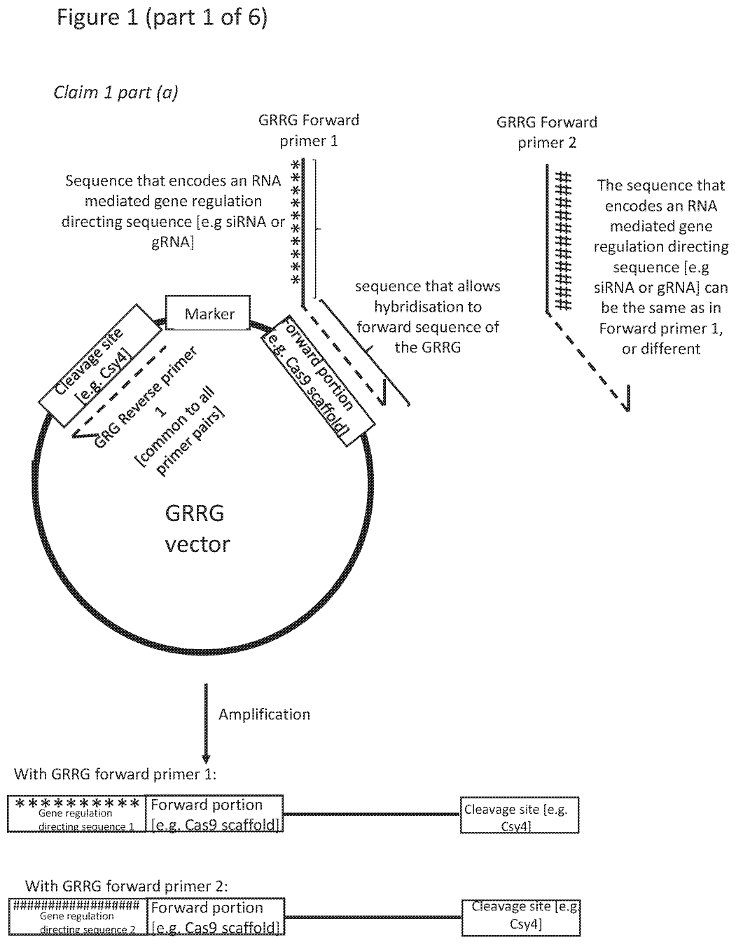

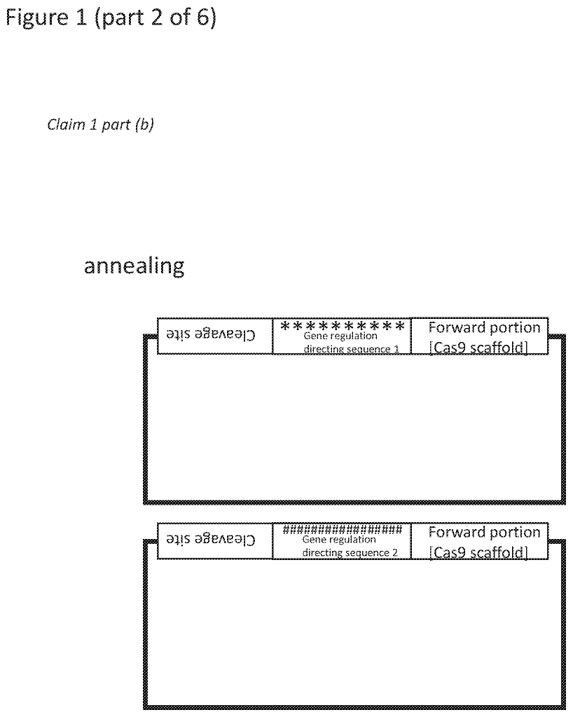

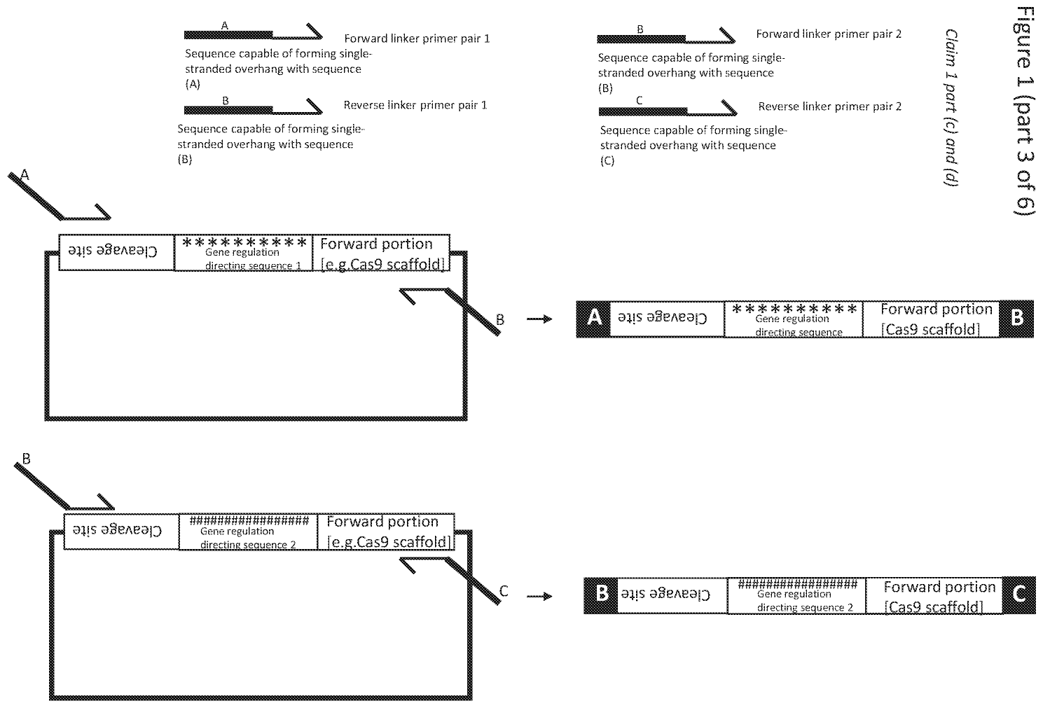

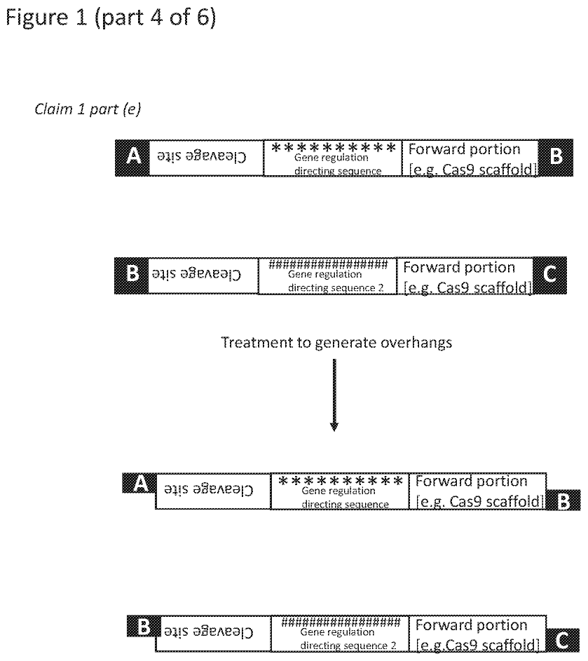

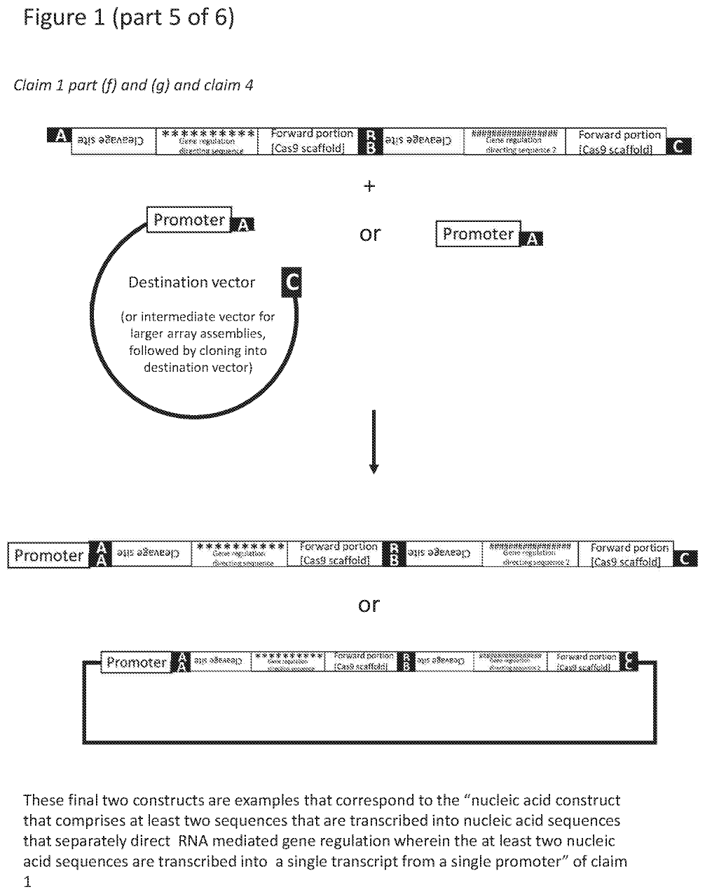

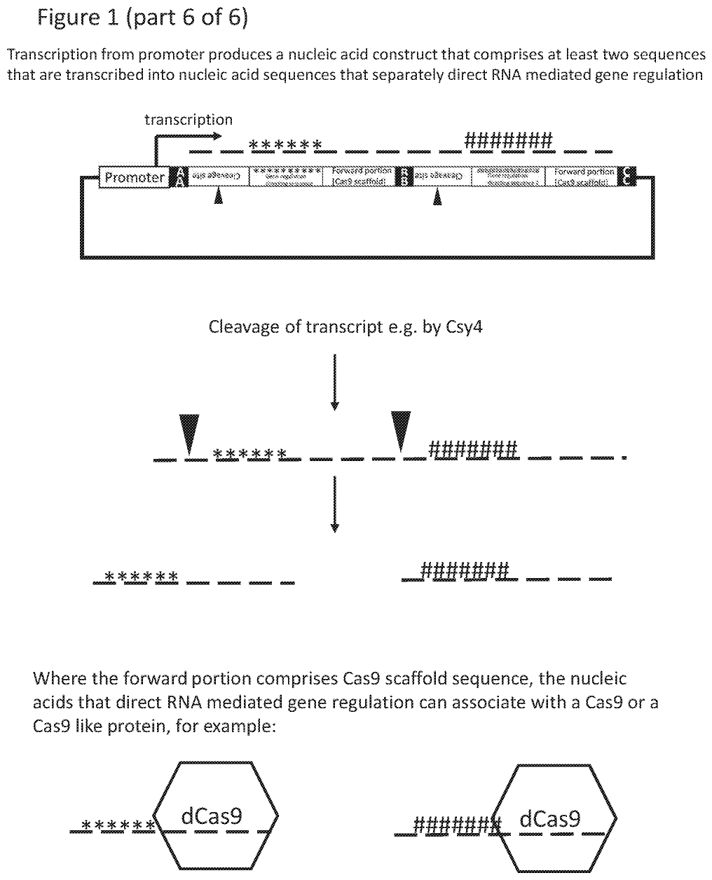

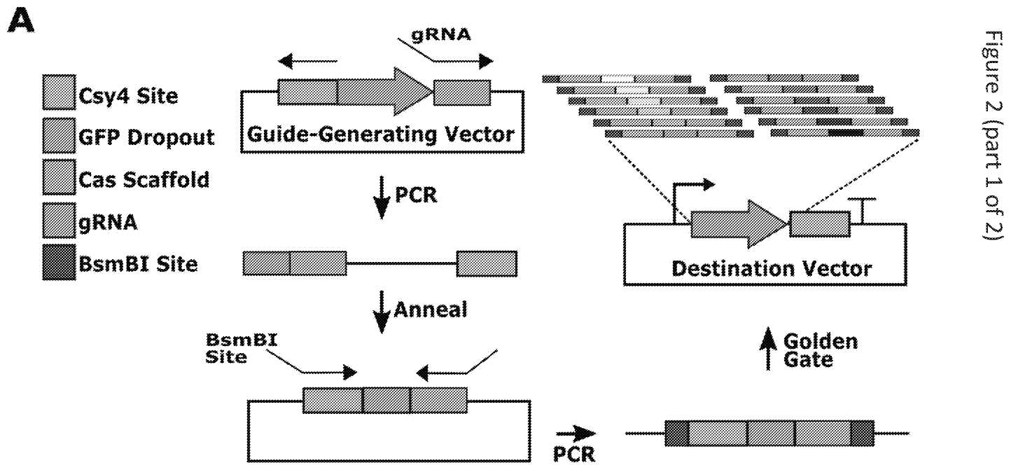

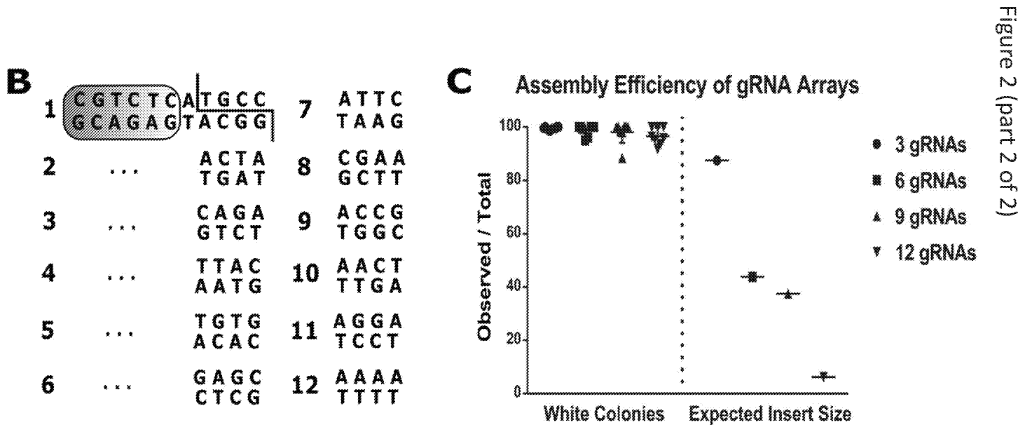

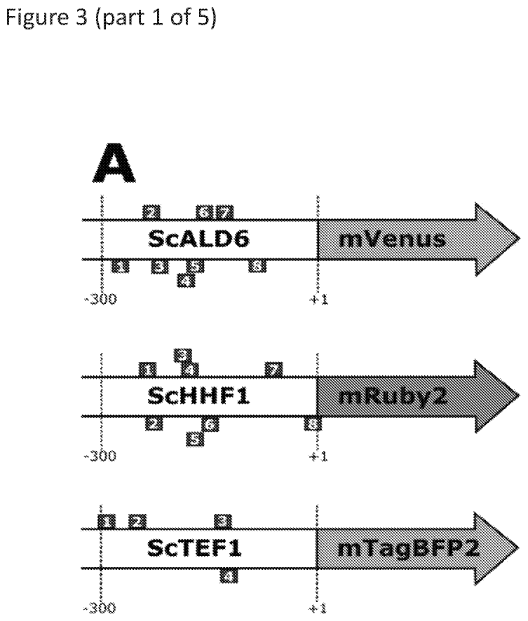

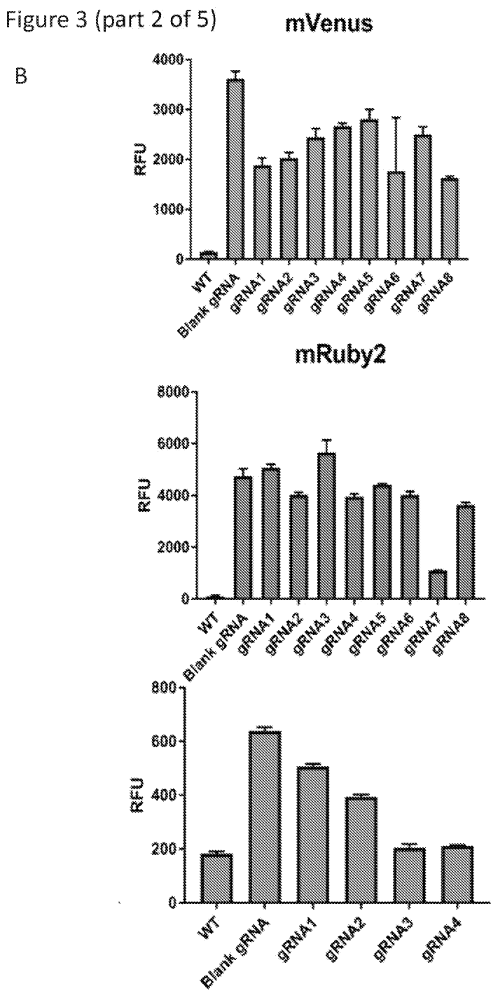

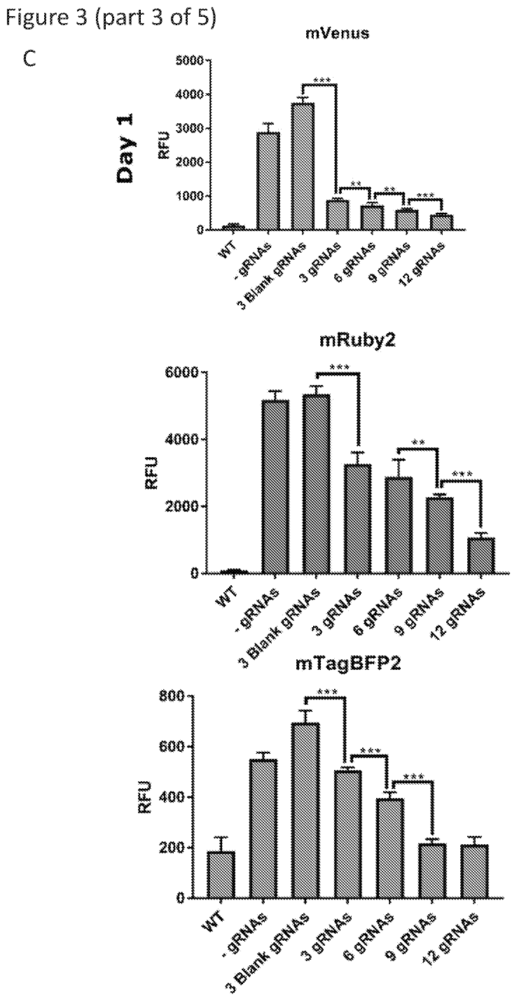

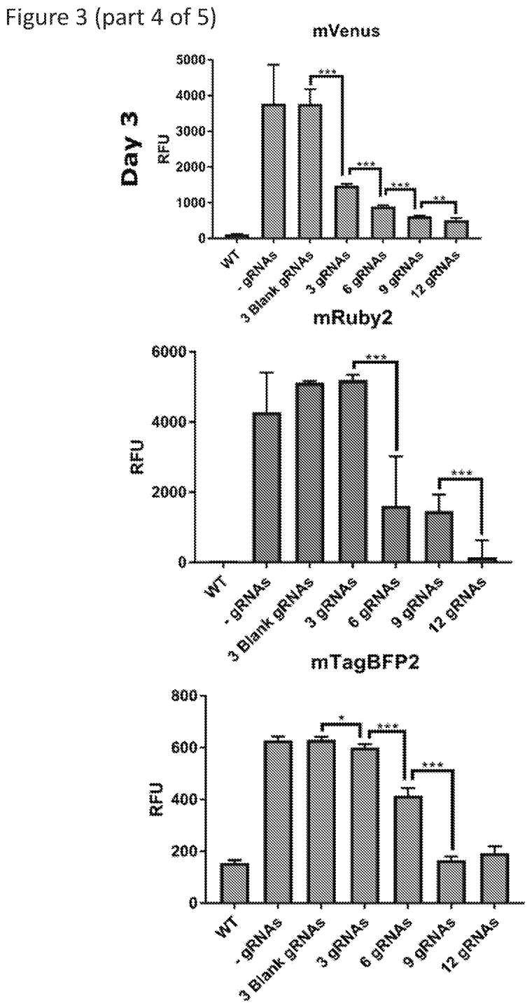

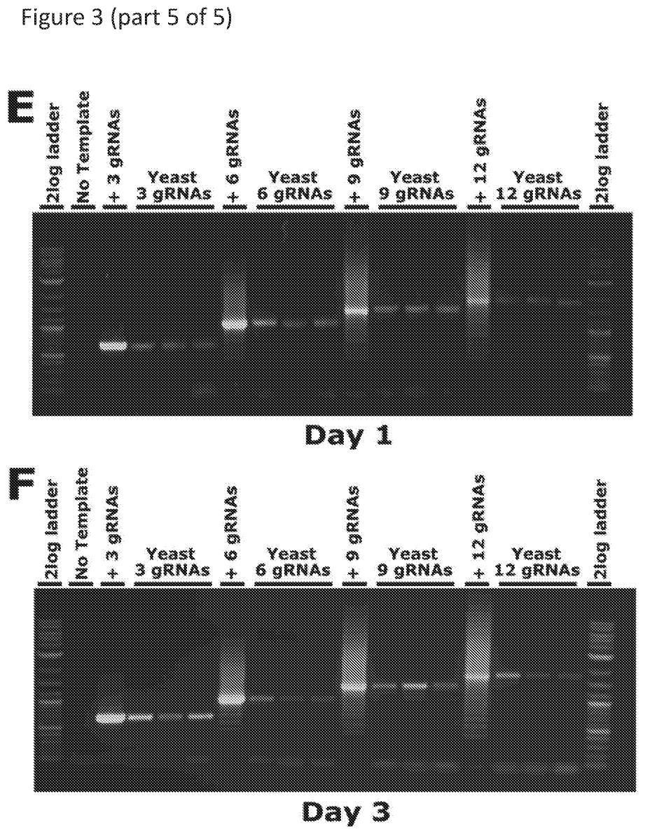

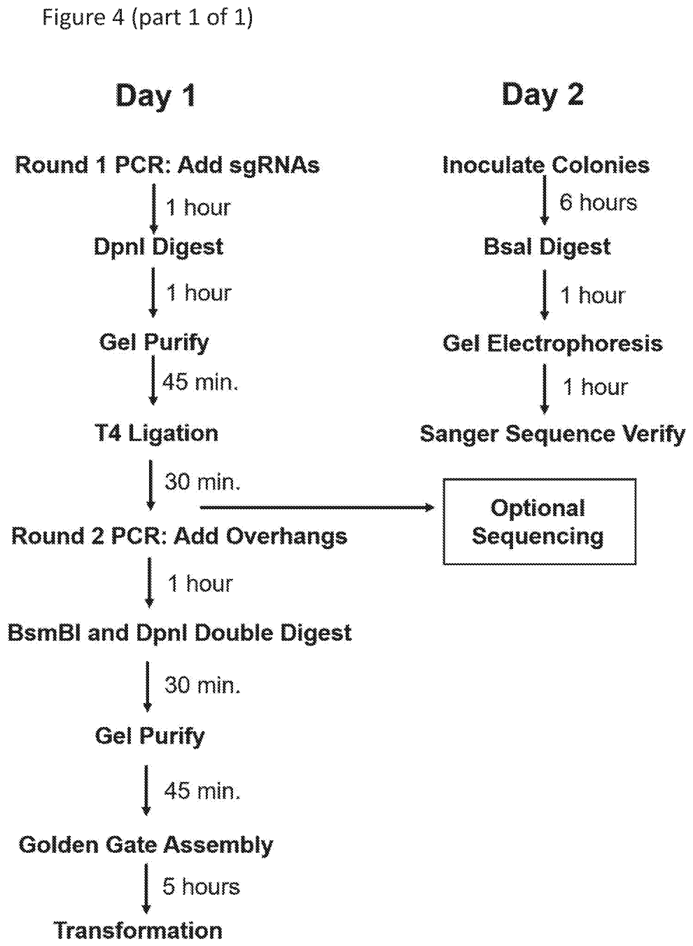

Once the DNA has been gel-purified, PCR fragments can be obtained that consist of our gRNA (5′ end of fragment), followed immediately by a Cas scaffold sequence, ColE1 and chloramphenicol resistance genes, and finally a Csy4 site on the 3′ end. By annealing these blunt-end, linear PCR fragments, a circularized vector is obtained that places the Csy4 site next to the gRNA targeting sequence and gRNA scaffold (see FIG. 1A in main text).

To Anneal the Isolated DNA Fragments:

TABLE 4
Ligation components to anneal PCR fragments
generated in Round 1.

| Component | Volume (μL) |
|---|---|
| T4 ligase buffer (NEB) | 1 |
| T4 DNA ligase (NEB) | 0.5 |
| 100 ng isolated DNA | Varies |
| Water (up to 10 μL total volume) | Varies |
| Reaction volume | 10 |

The annealing reaction mixtures were incubated at 37° C. for a minimum of 30 minutes.

Recommended, Optional Sequencing Step:

After obtaining circularized DNA vectors containing the gRNAs added via PCR, it is recommended that the DNA fragments be sequence-verified while simultaneously continuing with the next steps of the protocol. Sequencing is optional, and highly repetitive gRNA arrays can be constructed before sequence verification, but it is useful to have individual gRNA vectors be sequence-validated in case they are needed again later, in different constructs.

To sequence verify the DNA vectors with gRNAs, E. coli was transformed with each gRNA-containing vector and the cells were plated on LB agar with 1:1000 concentration of chloramphenicol.

After incubation at 37° C., colonies were picked and sent for Sanger sequencing, using the following primer, which binds in the ColE1 sequence of the annealed vector preceding the Csy4 site:

Primer for sequence verification of gRNA sequences in annealed vectors after Round 1 PCR—Forward Primer for sequencing of fragments after Round 1 PCR and isolation:

[SEQ ID NO. 25]
CTCACATGTTCTTTCCTGCG

After sending the annealed vectors containing the gRNA sequence for sequence validation, either wait for the sequencing results to be confirmed before proceeding (to ensure no contamination in round 1, which would be indicated by overlaps in peaks within the gRNA sequence regions in the chromatograms generated from Sanger sequencing) or continue immediately with the next stages of the CHORDS assembly protocol.

Round 2 PCR: Add BsmBI Overhangs

The next step is to add overhangs to each of the annealed vectors from the previous stages, which will enable their incorporation into a destination vector via BsmBI Golden Gate assembly. For this step, each PCR tube will contain a different template (the DNA vector with the gRNA sequences of interest) and a unique pair of forward and reverse primers, which are different than those used previously.

Round 2 PCR uses a small ‘library’ of primers that are fixed, meaning the primers can be ordered from an oligo manufacturer, for example, one time and then used repeatedly for CHORDS assembly. Each pair of primers adds a specific BsmBI recognition site and designed 4 bp overhang, which is compatible with the next gRNA in the final assembly. This enables the gRNAs generated in the previous steps to be placed in any position within the final transcript, simply by changing the primer pair used in this round for PCR.

The first gRNA in the array must always use the Position 1—Forward primer and the last gRNA in the array (whether an array is built with 5 gRNAs, 9 gRNAs, or 12 gRNAs, for example) must use the Position 12—Reverse primer.

List of primer pairs used in Round 2 PCR:

TABLE 5
Primer pairs for Round 2 PCR, which together add unigue BsmBI overhangs for
Golden Gate assembly.

| | | | 4bp | | SEQ |
|---|---|---|---|---|---|
| | Forward/ | | BsmBI | | ID |
| Position | Reverse | Sequence | Overhang | Note | NO: |
| 1 | Forwald | GCATCGTCTCATGCCgttcactgccgtataggcag | TGCC | Must always be used for | 26 |
| | | | | gRNA in first position. | |
| 1 | Reverse | ATGCCGTCTCATAGTaaaagcaccgactcggtg | | | 27 |
| 2 | Forward | GCATCGTCICAACTAgttcactgccataggcag | ACTA | | 28 |
| 2 | Reverse | ATGCCGTCTCATCTGaaaagcaccgactcGgtg | | | 29 |
| 3 | Forward | GCATCGTCTCACAGAgttcactgccgtataggcag | CAGA | | 30 |
| 3 | Reverse | ATGCCGTCTCAGTAAaaaagcaccgactcggtg | | | 31 |
| 4 | Forward | GCATCGTCTCATTACgttcactgccgtataggcag | TTAC | | 32 |
| 4 | Reverse | ATGCCGTCTCACACAaaaagcaccgactcggtg | | | 33 |
| 5 | Forward | GCATCGTCTCATGTGgttcactgccgtaggcag | TGTG | | 34 |
| 5 | Reverse | ATGCCGTCTCAGCTCaaaagcaccgactcggtg | | | 35 |
| 6 | Forward | GCATCGTCTCAGAGCgttcactgccgtataggcag | GAGC | | 35 |
| 6 | Reverse | ATGCCGTCTCAGAATaaaagcaccgactcggtg | | | 37 |
| 7 | Forward | GCATCGTCTCAATTCgttcactgccgtaggcag | ATTC | | 38 |
| 7 | Reverse | ATGCCGTCTCATTCGaaaagcaccgactcggtg | | | 39 |
| 8 | Forward | GCATCGTCTCACGAAgttcatgccgtataggcag | CGAA | | 40 |
| 8 | Reverse | ATGCCGTCTCACGGTaaaagcaccgactcggtg | | | 41 |
| 9 | Forward | GCATCGTCTCACCGgttcactgccgtataggcag | ACCG | | 42 |
| 9 | Reverse | ATGCCGTCTCAAGTTaaaagcaccgactcggtg | | | 43 |
| 10 | Forward | GCATCGTCTCAAACTgttcactgccgtataggcag | AACT | | 44 |
| 10 | Reverse | ATGCCGTCTCATCCTaaaagcaccgactcggtg | | | 45 |
| 11 | Forward | GCATCGTCTCAAAAAgttcactgccgtataggcag | AGGA | | 46 |
| 11 | Reverse | ATGCCGTCTCATTTTaaaagcaccgactcggtg | | | 47 |
| 12 | Forward | GCATCGTCTCAAAAAgttcactgccgtataggcag | AAAA | | 48 |
| 12 | Reverse | ATGCCGTCTCATTGCaaaagcaccgactcggtg | | Must always be used for | 49 |
| | | | | gRNA in termnal position | |

We report here are 12 different sets of primers, which enables up to 12 gRNAs to be assembled in a single array. However, these primer pairs are not limiting, and additional pairs could be designed to enable even longer gRNA arrays to be constructed. One of the only limitations regarding the number of gRNAs that can be assembled into a single array is considered to be the method used to join the gRNA sequences together, e.g. the Gold Gate reaction.

Once primer pairs were chosen (an example array assembly is provided in the next few paragraphs), the PCR reactions were setup with the different forward/reverse primer pairs and the unique, annealed guide-generating vector with the gRNA of interest, which was created in the previous steps.

To Set Up the PCR Reactions:

TABLE 6
PCR components for Round 2, which adds the BsmBI overhangs
for Golden Gate.

| Component | Volume (μL) |
|---|---|
| Nuclease-free water | 31 |
| 5 × Phusion HF Buffer | 10 |
| dNTPs (10 mM) | 1 |
| Forward Primer (10 μM) | 2.5 |
| Reverse Primer (10 μM) | 2.5 |
| Annealed Guide-Generating Vector w/ gR NA (10 ng/μL) | 1 |
| DMSO | 1.5 |
| Phusion Polymerase | 0.5 |
| Reaction volume | 50 |

Once the PCR tubes have been mixed, place samples in a thermocycler with the following settings (note the 61.3° C. annealing temperature):

TABLE 7
Thermocycler settings for Round 2 PCR.

| Step | Temp (° C.) | Time (s) |
|---|---|---|
| Initial Denaturation | 98 | 30 |
| 25-35 Cycles | 98 | 10 |
| | 61.3 | 30 |
| | 72 | 30 |
| Final Extension | 72 | 600 |
| Hold | 4 | |
| | PCR product | 150 bp |
| | length | |

Example of Primer Selection for Round 2 PCR:

In order to build a gRNA array with six unique gRNAs within a single transcriptional unit primer pairs for Round 2 PCR would be selected accordingly. It is essential that careful attention is paid to the selection of primer pairs, as these will ultimately add the 4 bp BsmBI overhangs that are crucial for Golden Gate assembly to create the final array in subsequent steps.

For the six-gRNA array, the following primers and templates indicated mar be used:

TABLE 8
Example primers to use to construct an array with six gRNAs with
CHORDS.

| PCR | | |
|---|---|---|
| Tube | Template DNA | Primers |
| #1 | Annealed Vector w/ gRNA for | Position 1 Forward, Position |
| | Position 1 in Array | 1 Reverse |
| #2 | Annealed Vector w/ gRNA for | Position 2 Forward, Position |
| | Position 2 in Array | 2 Reverse |
| #3 | Annealed Vector w/ gRNA for | Position 3 Forward, Position |
| | Position 3 in Array | 3 Reverse |
| #4 | Annealed Vector w/ gRNA for | Position 4 Forward, Position |
| | Position 4 in Array | 4 Reverse |
| #5 | Annealed Vector w/ gRNA for | Position 5 Forward, Position |
| | Position 5 in Array | 5 Reverse |
| #6 | Annealed Vector w/ gRNA for | Position 6 Forward, Position |
| | Position 6 in Array | 12 Reverse |

Note
the primer that is underlined—the gRNA in the final position must always use the Position 12 Reverse primer.

BsmBI and DpnI Double Digest:

After PCR, PCR tubes were removed, and a digestion was performed with restriction enzymes. If, for round 2 PCR, a template vector was used that had previously been transformed into E. coli, it will be necessary to digest the PCR mixture with DpnI and BsmBI.

If, for round 2 PCR, a template vector was used which had not been transformed into E. coli, it is necessary to digest the PCR mixture with BsmBI only.

To each PCR tube, 0.3 μL of each restriction enzyme was added. For a BsmBI/DpnI digest, samples were incubated at 37° C. for 30 minutes, followed by 55° C. for 30 minutes.

For a BsmBI digest, samples were incubated at 55° C. for 30 minutes.

A BsmBI digest was performed prior to gel purification to pre-digest the gRNA fragments. This step is thought to increase the efficiency of the Golden Gate reaction in subsequent steps.

Both BsmBI and DpnI retain activity in PCR buffers. See: https://www.neb.com/tools-and-resources/usage-guidelines/activity-of-restriction-enzymes-in-pcr-buffers

Gel Purify (2^{rd} Time):

The digest PCR samples were gel purified by performing agarose gel electrophoresis and gel extraction as described previously. In this second gel purification stage, it is not essential to spatially separate the DNA samples, as all extracted fragments will be added into the same Golden Gate reaction mixture in the steps that follow.

Golden Gate Reaction to Obtain the Final gRNA Array:

Once samples have been gel purified, their DNA concentration was determined via a NanoDrop machine. Each sample was diluted to 50 fmol for the Golden Gate reaction.

The Golden Gate reaction uses a plasmid backbone (which we term the Destination Vector) containing BsmBI sites, which the gRNA fragments with added BsmBI sites can be assembled into.

The Destination Vector used in this study consists of a promoter (the native yeast TDH3 promoter, for example), followed by a GFP gene (which is flanked by BsmBI sites and thus excised upon Golden Gate and a terminator (see FIG. 1a). Importantly, the Destination Vector also contains designed XhoI and BglII sites after the promoter and before the terminator, which enables any gRNA array, once assembled, to be swapped between different destination vectors.

The TDH3 destination vector used in this study will be made available on Addgene and its plasmid map can be viewed on Benchling. Simple instructions to create new destination vectors in a single day with Gibson Assembly is outlined later in this section.

While performing the Golden Gate reaction, all components were kept on ice and care was taken when pipetting. It is important to ensure that each part is diluted correctly, as this will increase the efficiency of the assembly.

To Set Up the Golden Gate Reaction:

TABLE 9
Components for the Golden Gate reaction, which is used to
assemble the final gRNA array.

| Component | Volume (μL) |
|---|---|
| 50 fmol Destination Vector | 0.15 |
| 50 fmol gRNAs + BsmBI overhangs (parts) | 0.5 (each) |
| T4 DNA ligase | 1 |
| 10 × T4 ligase buffer | 1 |
| BsmBI restriction enzyme | 1.5 |
| Water | Varies |
| Reaction volume | 10 |

Once the reaction mixture has been set up, the microtube was placed into a thermocycler using the following settings:

TABLE 10
Thermocycler settings for the Golden Gate reaction.

| Step | Temp (° C.) | Time (min) |
|---|---|---|
| 30 Cycles | 42 | 5 |
| | 16 | 5 |
| Incubation | 55 | 10 |
| Incubation #2 | 80 | 20 |
| Hold | 4 | ∞ |
| Size of Vector w/ gRNA | — | Destination Vector (bp) + |
| Array | | #gRNAs*150 bp |

Following the Golden Gate reaction, E. coli was transformed using a preferred method for cloning and streaked on LB agar plates with 1:1000 chloramphenicol.

The next day, white colonies were picked and prepared to screen for a colony containing the gRNA array of interest.

Screening for Correctly Assembled gRNA Arrays:

After picking white, single colonies of E. coli, cultures were inoculated in liquid LB with 1:1000 concentration of chloramphenicol at 37° C. for 6 hours. DNA purification (miniprep) was performed for stable extraction of plasmid DNA.

The destination vector utilized in the Golden Gate reaction contains BsaI restriction sites on the 5′ end of the promoter and 3′ end of the terminator, which enables straightforward screening of array size by BsaI digest.

Once a colony yielded an ‘expected’ band pattern following digestion with BsaI, it was essential that the putative plasmid be sequence-verified.

For gRNA arrays with 5 or less gRNAs, only one primer needs to be used (as the gRNA array is only about 750 bp in length). For gRNA arrays with 6 or more gRNAs, it is recommended that sequencing is performed with both a forward and reverse primer.

For gRNA arrays inserted into the destination vector with the TDH3 promoter and TDH1 terminator, the following primers may be used for sequencing:

Forward Primer (binds pTDH3)-
[SEQ ID NO: 50]
GACGGTAGGTATTGATTGTAATTC
Reverse Primer (binds tTDH1-
[SEQ ID NO: 51]
TGCTTAATCTTGTCTTGGCTTA

Assembly of Reporter and dCas9/Csy4 Constructs

Golden Gate was used to assemble vectors for genomic integration at the LEU2, HO or URA3 locus as described previously.¹⁰

Quantification of CHORDS Efficiency

50 μL TurboComp E. coli cells after CHORDS assembly and heat shock were streaked onto LB+chloramphenicol agar plates. GFP-negative and -positive colonies were counted manually with a blue light. 16 white colonies were randomly selected for each assembly condition and a BsaI restriction digest on 100 ng isolated DNA by adding 5 U of BsaI, 1 μL CutSmart buffer in a 10 μL reaction volume with water. Samples were incubated at 37° C. for 1 hour. The 10 μL reaction mixture was added to 2 μL of New England Biolabs 6× purple loading dye and loaded onto a 0.8% agarose gel in 1× TAE buffer at 100V for 40 minutes. Gels were imaged with blue light and an overhead camera in FluorChem software.

Flow Cytometry

Yeast transformant colonies were inoculated into liquid Synthetic Dropout media lacking the corresponding, auxotrophic amino acids and incubated in a 96-well, 2.2 mL deepwell plate at 30° C. and 700 rpm over a 5 day period. Every 12 hours, yeast were diluted in fresh media 1:100, with flow cytometry performed 6 hours after the second dilution each day. Cell fluorescence was measured by a BD LSRFortessa X-20 flow cytometer, with an attached BD HTS autosample. Fluorescence data was collected from 10,000 cells for each experiment and analyzed using FlowJo software. Flow cytometry settings: FSC sensor E01, SSC voltage 350, SSC threshold 52. mVenus excitation was with a green laser (532 nm) and detection via 530 nm filter. mRuby2 excitation was with a yellow/green laser (561 nm) and detection via a 590 nm filter. mTagBFP excitation was with a violet laser (405 nm) and detection via a 450 nm filter.

Colony PCR

Genomic DNA was isolated from yeast using the GC Preps protocol previously described.¹³ Before genomic DNA isolation, liquid yeast cultures were re-streaked onto Synthetic Dropout media and n=4 colonies picked for each condition at specified time points (either Day 1 or Day 5 of dilutions). Colony PCR was performed by adding 10 ng of the isolated genomic DNA to reaction mix containing 5 μL each of a forward (5′-gacggtaggtattgattgtaattc-3′ [SEQ ID NO: 50]) and reverse primer (5′-tgcttaatcttgtcttggctta-3′ [SEQ ID NO: 51]) (both 10 μM), 63 μL water, 20 μL 5× Phusion HF buffer, 2 μL dNTP mix (10 mM), 3 μL 100% DMSO and 1 μL high-fidelity Phusion polymerase. Thermocycler: 30 s denaturation at 98° C., 30 cycles of 98° C. for 10 s/59° C. for 30 s/72° C. for 30 s with final incubation at 72° C. for 10 min and hold at 4° C. Gel electrophoresis was performed as described above. References
(1) Cermak, T., Doyle, E. L., Christian, M., Wang, L., Zhang, Y., Schmidt, C., Bailer, J. A., Somia, N. V., Bogdanove, A. J., and Voytas, D. F. (2011) Erratum: Efficient design and assembly of custom TALEN and other TAL effector-based constructs for DNA targeting (Nucleic Acids Research (2011) 39 (e82) DOI: 10.1093/nar/gkr218). Nucleic Acids Res. 39, 7879.
(2) Jinek, M., Chylinski, K., Fonfara, I., Hauer, M., Doudna, J. A., and Charpentier, E. (2012) A Programmable Dual-RNA—Guided. Science 337, 816-822.
(3) Qi, L. S., Larson, M. H., Gilbert, L. A., Doudna, J. A., Weissman, J. S., Arkin, A. P., and Lim, W. A. (2013) Repurposing CRISPR as an RNA-guided platform for sequence-specific control of gene expression. Cell 152, 1173-1183.
(4) Didovyk, A., Borek, B., Tsimring, L., and Hasty, J. (2016) Transcriptional regulation with CRISPR-Cas9: Principles, advances, and applications. Curr. Opin. Biotechnol. 40, 177-184.
(5) Nowak, C. M., Lawson, S., Zerez, M., and Bleris, L. (2016) Guide RNA engineering for versatile Cas9 functionality. Nucleic Acids Res. 44, 9555-9564.
(6) Ferreira, R., Skrekas, C., Nielsen, J., and David, F. (2018) Multiplexed CRISPR/Cas9 Genome Editing and Gene Regulation Using Csy4 in Saccharomyces cerevisiae. ACS Synth. Biol. 7, 10-15.
(7) Kurata, M., Wolf, N. K., Lahr, W. S., Weg, M. T., Kluesner, M. G., Lee, S., Hui, K., Shiraiwa, M., Webber, B. R., and Moriarity, B. S. (2018) Highly multiplexed genome engineering using CRISPR/Cas9 gRNA arrays. PLoS One 13, e0198714.
(8) Jakočiunas, T., Jensen, M. K., and Keasling, J. D. (2016) CRISPR/Cas9 advances engineering of microbial cell factories. Metab. Eng. 34, 44-59.
(9) Hughes, R. A., and Ellington, A. D. (2017) Synthetic DNA Synthesis and Assembly: Putting the Synthetic in Synthetic Biology. Cold Spring Hart. Perspect. Biol. 9, a023812.
(10) Lee, M. E., DeLoache, W. C., Cervantes, B., and Dueber, J. E. (2015) A Highly Characterized Yeast Toolkit for Modular, Multipart Assembly. ACS Synth. Biol. 4, 975-986.
(11) Bzymek, M., and Lovett, S. T. (2001) Instability of repetitive DNA sequences: The role of replication in multiple mechanisms. Proc. Natl. Acad. Sci. 98, 8319-8325.
(12) Argueso, J. L., Westmoreland, J., Mieczkowski, P. A., Gawel, M., Petes, T. D., and Resnick, M. A. (2008) Double-strand breaks associated with repetitive DNA can reshape the genome. Proc. Natl. Acad. Sci. 105, 11845-11850.
(13) Blount, B. A., Driessen, M. R. M., and Ellis, T. (2016) GC preps: Fast and easy extraction of stable yeast genomic DNA. Sci. Rep. 6, 1-4.

### Example 3

In order to expand the number of DNA repetitive domains that can be assembled we have developed an additional step using Type IIS restriction enzymes (step (h)). The correct assembly becomes stochastically less probable with the increasing number of fragments assembled. Because of this, we have introduced additional hierarchy by assembling the domains in sets of up to 6. At least up to 4 of these sets may be joined in an additional step to reach 24 repetitive domains in total. It is considered preferable if no more than 7 fragments (for example, 1 backbone vector and 2-6 gRNA inserts) are assembled at each step, which keeps a high efficiency.

This additional step does not elongate the laboratory protocol. This is achieved by assembling the final array of repetitive domains directly into the vector that will be used for transformation, using a promoter and a marker of choice. The system is compatible most widely used toolkits of promoters and vectors to be used for regulation of the expression of the repetitive fragments.

Four intermediate vectors have been constructed to facilitate such longer arrays. See SEQ ID NO: 76-79. The partial arrays are assembled into these vectors. The choice of a vector depends on the position of the sub-array in the final assembly. As an example, four versions of a commonly used terminator tTDH1 have been constructed to allow for any length of the final array without spacers.

The workflow of the proposed methodology is as follows: the domains are designed as overhangs of a forward primer and assembled using PCR (using a stable reverse primer) and subsequent ligation into a guide generating vector. The original vector is digested by DpnI enzyme and also distinguished by expression of GFP in the host bacteria. This construct is optionally confirmed by sequencing. In the second round, PCR from this vector is conducted using a combination of primers that define the overhangs and hence the position in the array. The domain of interest is flanked by type IIS cut sites (as an example BsmBI) which will allow for specific overhangs used for the assembly. A reaction with a Type IIS restriction enzyme (as example BsmBI) and DNA ligase (as example T4) is set up to assemble up to 6 repetitive domains into one of the 4 intermediate vectors. The length of the inserts is confirmed by digestion or colony PCR. 1-4 of the filled intermediate vectors are used in a Type IIS restriction enzyme (as example BsaI) reaction with a final vector, promoter and terminator to create the final array. The length is confirmed by digestion of colony PCR.

As an example of application, this assembly has been demonstrated on arrays of gRNAs navigating Cas9 enzyme to its target. They have a repetitive structure where Csy4 cites are used to separate the gRNAs after transcription and a scaffold part repeats in every gRNA. The schematic of using the above described methodology for assembly of gRNAs is shown in FIG. 8.

### Example 4—Exemplary Vector Sequences, Highlighting the Different Components of Each Vector

[SEQ ID NO: 76] LOCUS pLS040_-_1st_acceptor_v 2680 bp ds-
DNA circular 22 MAY 2019

| DEFINITION . | |
|---|---|
| FEATURES | Location/Qualifiers |
| protein_bind | 1813..1818 |
| | /label=BsmBI″ |
| | /ApEinfo_revcolor=#b1ff67 |
| | /ApEinfo_fwdcolor=#b1ff67 |
| terminator | 1684..1812 |
| | /label=″BBa_B0015 Terminator″ |
| | /ApEinfo_revcolor=#b1ff67 |
| | /ApEinfo_fwdcolor=#b1ff67 |
| CDS | 967..1683 |
| | /label=″sfGFP″ |
| | /ApEinfo_revcolor=#b1ff67 |
| | /ApEinfo_fwdcolor=#b1ff67 |
| CDS | complement(1946..2605) |
| | /label=″CamR″ |
| | /ApEinfo_revcolor=#0000ff |
| | /ApEinfo_fwdcolor=#0000ff |
| promoter | 801..930 |
| | /label=″BBa_J72163 GlpT Promoter″ |
| | /ApEinfo_revcolor=#b1ff67 |
| | /ApEinfo_fwdcolor=#b1ff67 |
| RBS | 931..966 |
| | /label=″sfGFP Ribosome Binding Site″ |
| | /ApEinfo_revcolor=#b1ff67 |
| | /ApEinfo_fwdcolor=#b1ff67 |
| misc_feature | complement(1839..1945) |
| | /label=″CamR Terminator″ |
| | /ApEinfo_revcolor=#84b0dc |
| | /ApEinfo_fwdcolor=#84b0dc |
| rep_origin | complement(31..773) |
| | /label=″ColE1″ |
| | /ApEinfo_revcolor=#7f7f7f |
| | /ApEinfo_fwdcolor=#7f7f7f |
| promoter | complement(join(2606..2680,1..30)) |
| | /label=″CamR Promoter″ |
| | /ApEinfo_revcolor=#84b0dc |
| | /ApEinfo_fwdcolor=#84b0dc |
| protein_bind | complement(795..800) |
| | /label=″BsmBI″ |
| | /ApEinfo_revcolor=#b1ff67 |
| | /ApEinfo_fwdcolor=#b1ff67 |

ORIGIN

| | |
|---|---|
| 1 | aaagttggaa cctcttacgt gcccgatcaa tcatgaccaa aatcccttaa |
| | cgtgagtttt |
| 61 | cgttccactg agcgtcagac cccgtagaaa agatcaaagg atcttcttga |
| | gatccttttt |
| 121 | ttctgcgcgt aatctgctgc ttgcaaacaa aaaaaccacc gctaccagcg |
| | gtggtttgtt |
| 181 | tgccggatca agagctacca actctttttc cgaaggtaac tggcttcagc |
| | agagcgcaga |
| 241 | taccaaatac tgttcttcta gtgtagccgt agttaggcca ccacttcaag |
| | aactctgtag |
| 301 | caccgcctac atacctcgct ctgctaatcc tgttaccagt ggctgctgcc |
| | agtggcgata |
| 361 | agtcgtgtct taccgggttg gactcaagac gatagttacc ggataaggcg |
| | cagcggtcgg |
| 421 | gctgaacggg gggttcgtgc acacagccca gcttggagcg aacgacctac |
| | accgaactga |
| 481 | gatacctaca gcgtgagcta tgagaaagcg ccacgcttcc cgaagggaga |
| | aaggcggaca |
| 541 | ggtatccggt aagcggcagg gtcggaacag gagagcgcac gagggagctt |
| | ccagggggaa |
| 601 | acgcctggta tctttatagt cctgtcgggt ttcgccacct ctgacttgag |
| | cgtcgatttt |
| 661 | tgtgatgctc gtcagggggg gccagcaacg cggccttttt acggttcctg |
| | gccttttgct |
| 721 | ggccttttgc tcacatgttc tttcctgcgt tatcccctga ttctgtggat |
| | aaccgtaggg |
| 781 | tctcaTTCTC TGCcgagacg gaaagtgaaa cgtgatttca tgcgtcattt |
| | tgaacatttt |
| 841 | gtaaatctta tttaataatg tgtgcggcaa ttcacattta atttatgaat |
| | gttttcttaa |
| 901 | catcgcggca actcaagaaa cggcaggttc ggatcttagc tactagagaa |
| | agaggagaaa |
| 961 | tactagatgc gtaaaggcga agagctgttc actggtgtcg tccctattct |
| | ggtggaactg |
| 1021 | gatggtgatg tcaacggtca taagttttcc gtgcgtggcg agggtgaagg |
| | tgacgcaact |
| 1081 | aatggtaaac tgacgctgaa gttcatctgt actactggta aactgccggt |
| | tccttggccg |
| 1141 | actctggtaa cgacgctgac ttatggtgtt cagtgctttg ctcgttatcc |
| | ggaccatatg |
| 1201 | aagcagcatg acttcttcaa gtccgccatg ccggaaggct atgtgcagga |
| | acgcacgatt |
| 1261 | tcctttaagg atgacggcac gtacaaaacg cgtgcggaag tgaaatttga |
| | aggcgatacc |
| 1321 | ctggtaaacc gcattgagct gaaaggcatt gactttaaag aggacggcaa |
| | tatcctgggc |
| 1381 | cataagctgg aatacaattt taacagccac aatgtttaca tcaccgccga |
| | taaacaaaaa |
| 1441 | aatggcatta aagcgaattt taaaattcgc cacaacgtgg aggatggcag |
| | cgtgcagctg |
| 1501 | gctgatcact accagcaaaa cactccaatc ggtgatggtc ctgttctgct |
| | gccagacaat |
| 1561 | cactatctga gcacgcaaag cgttctgtct aaacctccga acgagaaacg |
| | cgatcatatg |
| 1621 | gttctgctgg agttcgtaac cgcagcgggc atcacgcatg gtatggatga |
| | actgtacaaa |
| 1681 | tgaccaggca tcaaataaaa cgaaaggctc agtcgaaaga ctgggccttt |
| | cgttttatct |
| 1741 | gttgtttgtc ggtgaacgct ctctactaga gtcacactgg ctcaccttcg |
| | ggtgggcctt |
| 1801 | tctgcgttta tacgtctctA TCCTGCCtga gaccagacca ataaaaaacg |
| | cccggcggca |
| 1861 | accgagcgtt ctgaacaaat ccagatggag ttctgaggtc attactggat |
| | ctatcaacag |
| 1921 | gagtccaagc gagctcgata tcaaattacg ccccgccctg ccactcatcg |
| | cagtactgtt |
| 1981 | gtaattcatt aagcattctg ccgacatgga agccatcaca aacggcatga |
| | tgaacctgaa |
| 2041 | tcgccagcgg catcagcacc ttgtcgcctt gcgtataata tttgcccatg |
| | gtgaaaacgg |
| 2101 | gggcgaagaa gttgtccata ttggccacgt ttaaatcaaa actggtgaaa |
| | ctcacccagg |
| 2161 | gattggctga aacgaaaaac atattctcaa taaacccttt agggaaatag |
| | gccaggtttt |
| 2221 | caccgtaaca cgccacatct tgcgaatata tgtgtagaaa ctgccggaaa |
| | tcgtcgtggt |
| 2281 | attcactcca gagcgatgaa aacgtttcag tttgctcatg gaaaacggtg |
| | taacaagggt |
| 2341 | gaacactatc ccatatcacc agctcaccgt ctttcattgc catacgaaat |
| | tccggatgag |
| 2401 | cattcatcag gcgggcaaga atgtgaataa aggccggata aaacttgtgc |
| | ttatttttct |
| 2461 | ttacggtctt taaaaaggcc gtaatatcca gctgaacggt ctggttatag |
| | gtacattgag |
| 2521 | caactgactg aaatgcctca aaatgttctt tacgatgcca ttgggatata |
| | tcaacggtgg |
| 2581 | tatatccagt gatttttttc tccattttag cttccttagc tcctgaaaat |
| | ctcgataact |
| 2641 | caaaaaatac gccoggtagt gatcttattt cattatggtg |
| | // |

[SEQ ID NO: 77] LOCUS pLS041_-_2nd acceptor_v 2680 bp ds-
DNA circular 6 JUN. 2019

| DEFINITION . | |
|---|---|
| FEATURES | Location/Qualifiers |
| promoter | 734..863 |
| | /label=″BBa_J72163 GlpT Promoter″ |
| | /ApEinfo_revcolor=#b1ff67 |
| | /ApEinfo_fwdcolor=#b1ff67 |
| CDS | 900..1616 |
| | /label=″sfGFP″ |
| | /ApEinfo_revcolor=#b1ff67 |
| | /ApEinfo_fwdcolor=#b1ff67 |
| terminator | 1617..1745 |
| | /label=″BBa_B0015 Terminator″ |
| | /ApEinfo_revcolor=#b1ff67 |
| | /ApEinfo_fwdcolor=#b1ff67 |
| protein_bind | 1746..1751 |
| | /label=″BsmBI″ |
| | /ApEinfo_revcolor=#b1ff67 |
| | /ApEinfo_fwdcolor=#b1ff67 |
| RBS | 864..899 |
| | /label=″sfGFP Ribosome Binding Site″ |
| | /ApEinfo_revcolor=#b1ff67 |
| | /ApEinfo_fwdcolor=#b1ff67 |
| rep_origin | complement(join(2644..2680,1..706)) |
| | /label=″ColE1″ |
| | /ApEinfo_revcolor=#7f7f7f |
| | /ApEinfo_fwdcolor=#7f7f7f |
| misc_feature | complement(1772-1878) |
| | /label=″CamR Terminator″ |
| | /ApEinfo_revcolor=#84b0dc |
| | /ApEinfo_fwdcolor=#84b0dc |
| CDS | complement(1879-2538) |
| | /label=″CamR″ |
| | /ApEinfo_revcolor=#0000ff |
| | /ApEinfo_fwdcolor=#0000ff |
| protein_bind | complement (728-733) |
| | /label=″BsmBi″ |
| | /ApEinfo_revcolor=#b1ff67 |
| | /ApEinfo_fwdcolor=#b1ff67 |
| promoter | complement(2539-2643) |
| | /label=″CamR Promoter″ |
| | /ApEinfo_revcolor=#84b0dc |
| | /ApEinfo_fwdcolor=#84b0dc |

ORIGIN

| | |
|---|---|
| 1 | ctgagcgtca gaccccgtag aaaagatcaa aggatcttct tgagatcctt |
| | tttttctgcg |
| 61 | cgtaatctgc tgcttgcaaa caaaaaaacc accgctacca gcggtggttt |
| | gtttgccgga |
| 121 | tcaagagcta ccaactcttt ttccgaaggt aactggcttc agcagagcgc |
| | agataccaaa |
| 181 | tactgttctt ctagtgtagc cgtagttagg ccaccacttc aagaactctg |
| | tagcaccgcc |
| 241 | tacatacctc gctctgctaa tccLgttacc agtggctgct gccagtggcg |
| | ataagtcgtg |
| 301 | tcttaccggg ttggactcaa gacgatagtt accggataag gcgcagcggt |
| | cgggctgaac |
| 361 | ggggggttcg tgcacacagc ccagcttgga gcgaacgacc tacaccgaac |
| | tgagatacct |
| 421 | acagcgtgag ctatgagaaa gcgccacgct tcccgaaggg agaaaggcgg |
| | acaggtatcc |
| 481 | ggtaagcggc agggtcggaa caggagagcg cacgagggag cttccagggg |
| | gaaacgcctg |
| 541 | gtatctttat agtcctgtcg ggtttcgcca cctctgactt gagcgtcgat |
| | ttttgtgatg |
| 601 | ctcgtcaggg ggggccagca acgcggcctt tttacggttc ctggcctttt |
| | gctggccttt |
| 661 | tgctcacatg ttctttcctg cgttatcccc tgattctgtg gataaccgta |
| | gggtctcaTG |
| 721 | CCCTGCcgag acggaaagtg aaacgtgatt tcatgcgtca ttttgaacat |
| | tttgtaaatc |
| 781 | ttatttaata atgtgtgcgg caattcacat ttaatttatg aatgttttct |
| | taacatcgcg |
| 841 | gcaactcaag aaacggcagg ttcggatctt agctactaga gaaagaggag |
| | aaatactaga |
| 901 | tgcgtaaagg cgaagagctg ttcactggtg tcgtccctat tctggtggaa |
| | ctggatggtg |
| 961 | atgtcaacgg tcataagttt tccgtgcgtg gcgagggtga aggtgacgca |
| | actaatggta |
| 1021 | aactgacgct gaagttcatc tgtactactg gtaaactgcc ggttccttgg |
| | ccgactctgg |
| 1081 | taacgacgct gacttatggt gttcagtgct ttgctcgtta tccggaccat |
| | atgaagcagc |
| 1141 | atgacttatt caagtccgcc atgccggaag gctatgtgca ggaacgcacg |
| | atttccttta |
| 1201 | aggatgacgg cacgtacaaa acgcgtgcgg aagtgaaatt tgaaggcgat |
| | accctggtaa |
| 1261 | accgcattga gctgaaaggc attgacttta aagaggacgg caatatcctg |
| | ggccataagc |
| 1321 | tggaatacaa ttttaacagc cacaatgttt acatcaccgc cgataaacaa |
| | aaaaatggca |
| 1381 | ttaaagcgaa ttttaaaatt cgccacaacg tggaggatgg cagcgtgcag |
| | ctggctcctc |
| 1441 | actaccagca aaacactcca atcggtgatg gtcctgttct gctgccagac |
| | aatcactatc |
| 1501 | tgagcacgca aagcgttctg tctaaagatc cgaacgagaa acgcgatcat |
| | atggttctgc |
| 1561 | tggagttcgt aaccgcagcg ggcatcacgc atggtatgga tgaactgtac |
| | aaatgaccag |
| 1621 | gcatcaaata aaacgaaagg ctcagtcgaa agactgggcc tttcgtttta |
| | tctgttgttt |
| 1681 | gtcggtgaac gctctctact agagtcacac tggctcacct tcgggtgggc |
| | ctttctgcgt |
| 1741 | ttatacgtct ctATCCCTAA tgagaccaga ccaataaaaa acgcccggcg |
| | gcaaccgagc |
| 1801 | gttctgaaca aatccagatg gagttctgag gtcattactg gatctatcaa |
| | caggagtcca |
| 1861 | agcgagctcg atatcaaatt acgccccgcc ctgccactca tcgcagtact |
| | gttgtaattc |
| 1921 | attaagcatt ctgccgacat ggaagccatc acaaacggca tgatgaacct |
| | gaatcgccag |
| 1981 | cggcatcagc accttgtcgc cttgcgtata atatttgccc atggtgaaaa |
| | cgggggcgaa |
| 2041 | gaagttgtcc atattggcca cgtttaaatc aaaactggtg aaactcaccc |
| | agggattggc |
| 2101 | tgaaacgaaa aacatattct caataaaccc tttagggaaa taggccaggt |
| | tttcaccgta |
| 2161 | acacgccaca tcttgcgaat atatgtgtag aaactgccgg aaatcgtcgt |
| | ggtattcact |
| 2221 | ccagagcgat gaaaacgttt cagtttgctc atggaaaacg gtgtaacaag |
| | ggtgaacact |
| 2281 | atcccatatc accagctcac cgtctttcat tgccatacga aattccggat |
| | gagcattcat |
| 2341 | caggcgggca agaatgtgaa taaaggccgg ataaaacttg tgcttatttt |
| | tctttacggt |
| 2401 | ctttaaaaag gccgtaatat ccagctgaac ggtctggtta taggtacatt |
| | gagcaactga |
| 2461 | ctgaaatgcc tcaaaatgtt ctttacgatg ccattgggat atatcaacgg |
| | tggtatatcc |
| 2521 | agtgattttt ttctccattt tagcttcctt agctcctgaa aatctcgata |
| | actcaaaaaa |
| 2581 | tacgcccggt agtgatctta tttcattatg gtgaaagttg gaacctctta |
| | cgtgcccgat |
| 2641 | caatcatgac caaaatccct taacgtgagt tttcgttcca |
| | // |

[SEQ ID NO: 78] LOCUS pLS042_-_3rd_acceptor_v 2680 bp ds-
DNA circular 11 APR. 2019

| DEFINITION . | |
|---|---|
| FEATURES | Location/Qualifiers |
| terminator | 2079..2207 |
| | /label=″BBa_B0015 Terminator″ |
| | /ApEinfo_revcolor=#b1ff67 |
| | /ApEinfo_fwdcolor=#b1ff67 |
| promoter | complement(321..425) |
| | /label=″CamR Promoter″ |
| | /ApEinfo_revcolor=#84b0dc |
| | /ApEinfo_fwdcolor=#84b0dc |
| CDS | 1362..2078 |
| | /label=″sfGFP″ |
| | /ApEinfo_revcolor=#b1ff67 |
| | /ApEinfo_fwdcolor=#b1ff67 |
| misc_feature | complement(2234..2340) |
| | /label=″CamR Terminator″ |
| | /ApEinfo_revcolor=#84b0dc |
| | /ApEinfo_fwdcolor=#84b0dc |
| protein_bind | 2208..2213 |
| | /label=″BsmBI″ |
| | /ApEinfo_revcolor=#b1ff67 |
| | /ApEinfo_fwdcolor=#b1ff67 |
| rep_origin | complement(426..1168) |
| | /label=″ColE1″ |
| | /ApEinfo_devcolor=#7f7f7f |
| | /ApEinfo_fwdcolor=#7f7f7f |
| RBS | 1326..1361 |
| | /label=″sfGFP Ribosome Binding Site″ |
| | /ApEinfo_revcolor=#b1ff67 |
| | /ApEinfo_fwdcolor=#b1ff67 |
| promoter | 1196..1325 |
| | /label=″BBa_J72163 GlpT Promoter″ |
| | /ApEinfo_revcolor=#b1ff67 |
| | /ApEinfo_fwdcolor=#b1ff67 |
| CDS | complement(join(2341..2680,1..320)) |
| | /label=″CamR″ |
| | /ApEinfo_revcolor=#0000ff |
| | /ApEinfo_fwdcolor=#0000ff |
| protein_bind | complement(1190..1195) |
| | /label=″BsmBI″ |
| | /ApEinfo_devcolor=#b1ff67 |
| | /ApEinfo_fwdcolor=#b1ff67 |

ORIGIN

| | |
|---|---|
| 1 | ctccagagcg atgaaaacgt ttcagtttgc tcatggaaaa cggtgtaaca |
| | agggtgaaca |
| 61 | ctatcccata tcaccagctc accgtctttc attgccatac gaaattccgg |
| | atgagcattc |
| 121 | atcaggcggg caagaatgtg aataaaggcc ggataaaact tgtgcttatt |
| | tttctttacg |
| 181 | gtctttaaaa aggccgtaat atccagctga acggtctggt tataggtaca |
| | ttgagcaact |
| 241 | gactgaaatg cctcaaaatg ttctttacga tgccattggg atatatcaac |
| | ggtggtatat |
| 301 | ccagtgattt ttttctccat tttagcttcc ttagctcctg aaaatctcga |
| | taactcaaaa |
| 361 | aatacgcccg gtagtgatct tatttcatta tggtgaaagt tggaacctct |
| | tacgtgcccg |
| 421 | atcaatcatg accaaaatcc cttaacgtga gttttcgttc cactgagcgt |
| | cagaccccgt |
| 481 | agaaaagatc aaaggatctt cttgagatcc tttttttctg cgcgtaatct |
| | gctgcttgca |
| 541 | aacaaaaaaa ccaccgctac cagcggtggt ttgtttgccg gatcaagagc |
| | taccaactct |
| 601 | ttttccgaag gtaactggct tcagcagagc gcagatacca aatactgttc |
| | ttctagtgta |
| 661 | gccgtagtta ggccaccact tcaagaactc tgtagcaccg cctacatacc |
| | tcgctctgct |
| 721 | aatcctgtta ccagtggctg ctgccagtgg cgataagtcg tgtcctaccg |
| | ggttggactc |
| 781 | aagacgatag ttaccggata aggcgcagcg gtcgggctga acggggggtt |
| | cgtgcacaca |
| 841 | gcccagcttg gagcgaacga cctacaccga actgagatac ctacagcgtg |
| | agctatgaga |
| 901 | aagcgccacg cttcccgaag ggagaaaggc ggacaggtat ccggtaagcg |
| | gcagggtcgg |
| 961 | aacaggagag cgcacgaggg agcttccagg gggaaacgcc tggtatcttt |
| | atagtcctgt |
| 1021 | cgggtttcgc cacctctgac ttgagcgtcg atttttgcga tgctcgtcag |
| | ggggggccag |
| 1081 | caacgcggcc tttttacggt tcctggcctt ttgctggcct tttgctcaca |
| | tgttctttcc |
| 1141 | tgcgttatcc cctgattctg tggataaccg tagggtctca CTAACTGCcg |
| | agacggaaag |
| 1201 | tgaaacgtga tttcatgcgt cattttgaac attttgtaaa tcttatttaa |
| | taatgtgtgc |
| 1261 | ggcaattcac atttaattta tgaatgtttt cttaacatcg cggcaactca |
| | agaaacggca |
| 1321 | ggttcggatc ttagctacta gagaaagagg agaaatacta gatgcgtaaa |
| | ggcgaagagc |
| 1381 | tgttcactgg tgtcgtccct attctggtgg aactggaagg tgatgtcaac |
| | ggtcataagt |
| 1441 | tttccgtgcg tggcgagggt gaaggtgacg caactaatgg taaactgacg |
| | ctgaagttca |
| 1501 | tctgtactac tggtaaactg ccggttcctt ggccgactct ggtaacgacg |
| | ctgacttatg |
| 1561 | gtgttcagtg ctttgctcgt tatccggacc atatgaagca gcatgacttc |
| | ttcaagtccg |
| 1621 | ccatgccgga aggctatgtg caggaacgca cgatttcctt taaggatgac |
| | ggcacgtaca |
| 1681 | aaacgcgtgc ggaagtgaaa tttgaaggcg ataccctggt aaaccgcatt |
| | gagctgaaag |
| 1741 | gcattgactt taaagaggac ggcaatatcc tgggccataa gctggaatac |
| | aattttaaca |
| 1801 | gccacaatgt ttacatcacc gccgataaac aaaaaaatgg cattaaagcg |
| | aattttaaaa |
| 1861 | ttcgccacaa cgtggaggat ggcagcgtgc agctggctga tcactaccaa |
| | caaaacactc |
| 1921 | caatcggtga tggtcctgtt ctgctgccag acaatcacta tctgagcacg |
| | caaagcgttc |
| 1981 | tgtctaaaga tccgaacgag aaacgcgatc atatggttct gctggagttc |
| | gtaaccgcag |
| 2041 | cgggcatcac gcatggtatg gatgaactgt acaaatgacc aggcatcaaa |
| | taaaacgaaa |
| 2101 | ggctcagtcg aaagactggg cctttcgttt tatctgttgt ttgtcggtga |
| | acgctctcta |
| 2161 | ctagagtcac actggctcac cttcgggtgg gcctttctgc gtttatacgt |
| | ctctATCCAC |
| 2221 | CAtgagacca gaccaataaa aaacgcccgg cggcaaccga gcgttctgaa |
| | caaatccaga |
| 2281 | tggagttctg aggtcattac tggatctatc aacaggagtc caagcgagct |
| | cgatatcaaa |
| 2341 | ttacgccccg ccctgccact catcgcagta ctgttgtaat tcattaagca |
| | ttctgccgac |
| 2401 | atggaagcca tcacaaacgg catgatgaac ctgaatcgcc agcggcatca |
| | gcaccttgtc |
| 2461 | gccttgcgta taatatttgc ccatggtgaa aacgggggcg aagaagttgt |
| | ccatattggc |
| 2521 | cacgtttaaa tcaaaactgg tgaaactcac ccagggattg gctgaaacga |
| | aaaacatatt |
| 2581 | ctcaataaac cctttaggga aataggccag gttttcaccg taacacgcca |
| | catcttgcga |
| 2641 | at-tatgtgt agaaactgcc ggaaatcgtc gtggtaLtca |
| | // |

[SEQ ID NO: 79] LOCUS pLS043_-_4th_acceptor_v 2680 bp ds-
DNA circular 11 APR. 2019

| DEFINITION . | |
|---|---|
| FEATURES | Location/Qualifiers |
| RBS | 355..390 |
| | /label=″sfGFP Ribosome Binding Site″ |
| | /ApEinfo_revcolor=#b1ff67 |
| | /ApEinfo_fwdcolor=#b1ff67 |
| promoter | 225..354 |
| | /label=″BBa_J72163 GlpT Promoter″ |
| | /ApEinfo_revcolor=#b1ff67 |
| | /ApEinfo_fwdcolor=#b1ff67 |
| promoter | complement(2030..2134) |
| | /label=″CamR Promoter″ |
| | /ApEinfo_revcolor=#84b0dc |
| | /ApEinfo_fwdcolor=#84b0dc |
| protein_bind | complement(219..224) |
| | /label=″BsmBI″ |
| | /ApEinfo_revcolor=#b1ff67 |
| | /ApEinfo_fwdcolor=#b1ff67 |
| CDS | complement(1370..2029) |
| | /label=″CamR″ |
| | /ApEinfo_revcolor=#0000ff |
| | /ApEinfo_fwdcolor=#0000ff |
| terminator | 1108..1236 |
| | /label=″BBa_B0015 Terminator″ |
| | /ApEinfo_revcolor=#b1ff67 |
| | /ApEinfo_fwdcolor=#b1ff67 |
| CDS | 391..1107 |
| | /label=″sfGFP″ |
| | /ApEinfo_revcolor=#b1ff67 |
| | /ApEinfo_fwdcolor=#b1ff67 |
| misc_feature | complement(1263..1369) |
| | /label=″CamR Terminator″ |
| | /ApEinfo_revcolor=#84b0dc |
| | /ApEinfo_fwdcolor=#84b0dc |
| rep_origin | complement(join(2135..2680,1..197)) |
| | /label=″ColE1″ |
| | /ApEinfo_revcolor=#7f7f7f |
| | /ApEinfo_fwdcolor=#7f7f7f |
| protein_bind | 1237..1242 |
| | /label=″BsmBI″ |
| | /ApEinfo_revcolor=#b1ff67 |
| | /ApEinfo_fwdcolor=#b1ff67 |

ORIGIN

| | |
|---|---|
| 1 | gcacgaggga gcttccaggg ggaaacgcct ggtatcttta tagtcctgtc |
| | gggtttcgcc |
| 61 | acctctgact tgagcgtcga tttttgtgat gctcgtcagg gggggccagc |
| | aacgcggcct |
| 121 | ttttacggtt cctggccttt tgctggcctt ttgctcacat gttctttcct |
| | gcgttatccc |
| 181 | ctgartctgt ggataaccgt agggtctcaA CCACTGCcga gacggaaagt |
| | gaaacgtgat |
| 241 | ttcatgcgtc attttgaaca ttttgtaaat cttatttaat aatgtgtgcg |
| | gcaattcaca |
| 301 | tttaatttat gaatgttttc ttaacatcgc ggcaactcaa gaaacggcag |
| | gttcggatct |
| 361 | tagctactag agaaagagga gaaatactag atgcgtaaag gcgaagagct |
| | gttcactggt |
| 421 | gtcgtcccta ttctggtgga actggatggt gatgtcaacg gtcataagtt |
| | ttccgtgcgt |
| 481 | ggcgagggtg aaggtgacgc aactaatggt aaactgacgc tgaagttcat |
| | ctgtactact |
| 541 | ggtaaactgc cggttccttg gccgactctg gtaacgacgc tgacttatgg |
| | tgttcagtgc |
| 601 | tttgctcgtt atccggacca tatgaagcag catgacttct tcaagtccgc |
| | catgccggaa |
| 661 | ggctatgtgc aggaacgcac gatttccttt aaggatgacg gcacgtacaa |
| | aacgcgtgcg |
| 721 | gaagtgaaat ttgaaggcga taccctggta aaccgcattg agctgaaagg |
| | cattgacttt |
| 781 | aaagaggacg gcaatatcct gggccataag ctggaataca attttaacag |
| | ccacaatgtt |
| 841 | tacatcaccg ccgataaaca aaaaaatggc attaaagcga attttaaaat |
| | tcgccacaac |
| 901 | gtggaggatg gcagcgtgca gctggctgat cactaccagc aaaacactcc |
| | aatcggtgat |
| 961 | ggtcctgttc tgctgccaga caatcactat ctgagcacgc aaagcgttct |
| | gtctaaagat |
| 1021 | ccgaacgaga aacgcgatca tatggttctg ctggagttcg taaccgcagc |
| | gggcatcacg |
| 1081 | catggtatgg atgaactgta caaatgacca ggcatcaaat aaaacgaaag |
| | gctcagtcga |
| 1141 | aagactgggc ctttcgtttt atctgttgtt tgtcggtgaa cgctctctac |
| | tagagtcaca |
| 1201 | ctggctcacc ttcgggtggg cctttctgcg tttatacgtc tctATCCATC |
| | Ctgagaccag |
| 1261 | accaataaaa aacgcccggc ggcaaccgag cgttctgaac aaatccagat |
| | ggagttctga |
| 1321 | ggtcattact ggatctatca acaggagtcc aagcgagctc gatatcaaat |
| | tacgccccgc |
| 1381 | cctgccactc atcgcagtac tgttgtaatt cattaagcat tctgccgaca |
| | tggaagccat |
| 1441 | cacaaacggc atgatgaacc tgaatcgcca gcggcatcag caccttgtcg |
| | ccttgcgtat |
| 1501 | aatatttgcc catggtgaaa acgggggcga agaagttgtc catattggcc |
| | acgtttaaat |
| 1561 | caaaactggt gaaactcacc cagggattgg ctgaaacgaa aaacatattc |
| | tcaacaaacc |
| 1621 | ctttagggaa ataggccagg ttttcaccgt aacacgccac atcttgcgaa |
| | tatatgtgta |
| 1681 | gaaactgccg gaaatcgtcg tggtattcac tccagagcga tgaaaacgtt |
| | tcagtttgct |
| 1741 | catggaaaac ggtgtaacaa gggtgaacac tatcccatat caccagctca |
| | ccgtctttca |
| 1801 | ttgccatacg aaattccgga tgagcattca tcaggcgggc aagaatgtga |
| | ataaaggccg |
| 1861 | gataaaactt gtgcttattt ttctttacgg tctttaaaaa ggccgtaata |
| | tccagctgaa |
| 1921 | cggtctggtt ataggtacat tgagcaactg actgaaatgc ctcaaaatgt |
| | tctttacgat |
| 1981 | gccattggga tatatcaacg gtggtatatc cagtgatttt tttctccatt |
| | ttagcttcct |
| 2041 | tagctcctga aaatctcgat aactcaaaaa atacgcccgg tagtgatctt |
| | atttcattat |
| 2101 | ggtgaaagtt ggaacctctt acgtgcccga tcaatcatga ccaaaatccc |
| | ttaacgtgag |
| 2161 | ttttcgttcc actgagcgtc agaccccgta gaaaagatca aaggatcttc |
| | ttgagatcct |
| 2221 | ttttttctgc gcgtaatctg ctgcttgcaa acaaaaaaac caccgctacc |
| | agcggtggtt |
| 2281 | tgtttgccgg atcaagagct accaactctt tttccgaagg taactggctt |
| | cagcagagcg |
| 2341 | cagataccaa atactgttct tctagtgtag ccgtagttag gccaccactt |
| | caagaactct |
| 2401 | gtagcaccgc ctacatacct cgctctgcta atcctgttac cagtggctgc |
| | tgccagtggc |
| 2461 | gataagtcgt gtcttaccgg gttggactca agacgatagt taccggataa |
| | ggcgcagcgg |
| 2521 | tcgggctgaa cggggggttc gtgcacacag cccagcttgg agcgaacgac |
| | ctacaccgaa |
| 2581 | ctgagatacc tacagcgtga gctatgagaa agcgccacgc ttcccgaagg |
| | gagaaaggcg |
| 2641 | gacaggtatc cggtaagcgg cagggtcgga acaggagagc |
| | // |

[SEQ ID NO: 80] LOCUS pLS039_-_pTDH3_with_TTC 2351 bp ds-
DNA circular 5 JUN. 2019

| DEFINITION . | |
|---|---|
| FEATURES | Location/Qualifiers |
| CDS | complement(join(2095..2351,1..403)) |
| | /label=″CamR″ |
| | /ApEinfo_revcolor=#0000ff |
| | /ApEinfo_fwdcolor=#0000ff |
| protein_bind | complement(1978..1983) |
| | label=″BsaI″ |
| | /ApEinfo_revcolor=#b1ff67 |
| | /ApEinfo_fwdcolor=#b1ff67 |
| protein_bind | 1277..1282 |
| | /label=″BsaI″ |
| | /ApEinfo_revcolor=#b1ff67 |
| | /ApEinfo_fwdcolor=#b1ff67 |
| promoter | 1288..1967 |
| | /label=″ScTDH3 Promoter″ |
| | /ApEinfo_revcolor=#84b0dc |
| | /ApEinfo_fwdcolor=#84b0dc |
| protein_bind | 1284..1287 |
| | /label=″BsaI″ |
| | /ApEinfo_revcolor=#b1ff67 |
| | /ApEinfo_fwdcolor=#b1ff67 |
| terminator | complement(1986..2094) |
| | /label=″CamR Terminator″ |
| | /ApEinfo_revcolor=#84b0dc |
| | /ApEinfo_fwdcolor=#84b0dc |
| rep_origin | complement(509..1272) |
| | /label=″ColE1″ |
| | /ApEinfo_revcolor=#7f7f7f |
| | /ApEinfo_fwdcolor=#7f7f7f |
| promoter | complement(404..508) |
| | /label=″CamR Promoter″ |
| | /ApEinfo_revcolor=#84b0dc |
| | /ApEinfo_fwdcolor=#84b0dc |

ORIGIN

| | |
|---|---|
| 1 | ggaaataggc caggttttca ccgtaacacg ccacatcttg cgaatatatg |
| | tgtagaaact |
| 61 | gccggaaatc gtcgtggtat tcactccaga gcgatgaaaa cgtttcagtt |
| | tgctcatgga |
| 121 | aaacggtgta acaagggtga acactatccc atatcaccag ctcaccgtct |
| | ttcattgcca |
| 181 | tacgaaattc cggatgagca ttcatcaggc gggcaagaat gtgaataaag |
| | gccggataaa |
| 241 | acttgtgctt atttttcttt acggtcttta aaaaggccgt aatatccagc |
| | tgaacggtct |
| 301 | ggttataggt acattgagca actgactgaa atgcctcaaa atgttcttta |
| | cgatgccatt |
| 361 | gggatatatc aacggtggta tatccagtga tttttttctc cattttagct |
| | tccttagctc |
| 421 | ctgaaaatct cgataactca aaaaatacgc ccggtagtga tcttatttca |
| | ttatggtgaa |
| 481 | agttggaacc tcttacgtgc ccgatcaatc atgaccaaaa tcccttaacg |
| | tgagttttcg |
| 541 | ttccactgag cgtcagaccc cgtagaaaag atcaaaggat cttcttgaga |
| | tccttttttt |
| 601 | ctgcgcgtaa tctgctgctt gcaaacaaaa aaaccaccgc taccagcggt |
| | ggtttgtttg |
| 661 | ccggatcaag agctaccaac tctttttccg aaggtaactg gcttcagcag |
| | agcgcagata |
| 721 | ccaaatactg ttcttctagt gtagccgtag ttaggccacc acttcaagaa |
| | ctctgtagca |
| 781 | ccgcctacat acctcgctct gctaatcctg ttaccagtgg ctgctgccag |
| | tggcgataag |
| 841 | tcgtgtctta ccgggttgga ctcaagacga cagttaccgg ataaggcgca |
| | gcggtcgggc |
| 901 | tgaacggggg gttcgtgcac acagcccagc ttggagcgaa cgacctacac |
| | cgaactgaga |
| 961 | tacctacagc gtgagctatg agaaagcgcc acgattcccg aagggagaaa |
| | ggcggacagg |
| 1021 | tatccggtaa gcggcagggt cggaacagga gagcgcacga gggagcttcc |
| | agggggaaac |
| 1081 | gcctggtatc tttatagtcc tgtcgggttt cgccacctct gacttgagcg |
| | tcgatttttg |
| 1141 | tgatgctcgt caggggggcg gagcctatgg aaaaacgcca gcaacgcggc |
| | ctttttacgg |
| 1201 | ttcctggcct tttgctggcc ttttgctcac atgttctttc ctgcgttatc |
| | ccctgattct |
| 1261 | gtggataacc gtagtcggtc tcaaacgcag ttcgagttta tcattatcaa |
| | tagtgccatt |
| 1321 | tcaaagaata cgtaaataat taatagtagt gattttccta actttattta |
| | gtcaaaaaat |
| 1381 | tagcctttta attctgctgt aacccgtaca tgcccaaaat agggggcggg |
| | ttacacagaa |
| 1441 | tatataacat cgtaggtgtc tgggtgaaca gtttattcct ggcatccact |
| | aaatataatg |
| 1501 | gagcccgctt tttaagctgg catccagaaa aaaaaagaat cccagcacca |
| | aaatattgtt |
| 1561 | ttcttcacca accatcagtt cataggtcca ttctcttagc gcaactacag |
| | agaacagggg |
| 1621 | cacaaacagg caaaaaacgg gcacaacctc aatggagtga tgcaacctgc |
| | ctggagtaaa |
| 1681 | tgatgacaca aggcaattga cccacgcatg tatctatctc attttcttac |
| | accttctatt |
| 1741 | accttctgct ctctctgatt tggaaaaagc tgaaaaaaaa ggttgaaacc |
| | agttccctga |
| 1801 | aattattccc ctacttgact aataagtata taaagacggt aggtattgat |
| | tgtaattctg |
| 1861 | taaatctatt tcttaaactt cttaaattct acttttatag ttagtctttt |
| | ttttagtttt |
| 1921 | aaaacaccaa gaacttagtt tcgaataaac acacataaac aaacaaaaga |
| | tcTTCTtgag |
| 1981 | accagaccaa taaaaaacgc ccggcggcaa ccgagcgttc tgaacaaatc |
| | cagatggagt |
| 2041 | tctgaggtca ttagtggatc tatcaacagg agtccaagcg agctcgatat |
| | caaattacgc |
| 2101 | cccgccctgc cactcatcgc agtactgttg taattcatta agcattctgc |
| | cgacatggaa |
| 2161 | gccatcacaa acggcatgat gaacctgaat cgccagcggc atcagcacct |
| | tgtcgccttg |
| 2221 | cgtataatat ttgcccatgg tgaaaacggg ggcgaagaag ttgtccatat |
| | tggccacgtt |
| 2281 | taaatcaaaa ctggtgaaac tcacccaggg attggctgaa acgaaaaaca |
| | tattctcaat |
| 2341 | aaacccttta g |

[SEQ ID NO: 81] LOCUS pLS070_-_tTDH1)_[4] _modi 1915 bp ds-
DNA circular 15 JUN. 2019

| DEFINITION . | ″E. coli Marker: CamR″ |
|---|---|
| KEYWORDS | ″Seguence Verified″ ″Type: 4″ |
| FEATURES | Location/Qualifiers |
| terminator | 1570..1793 |
| | /label=″ScTDH1 Terminator″ |
| | /ApEinfo_revcolor=#ff9ccd |
| | /ApEinfo_fwdcolor=#ff9ccd |
| protein_bind | complement(1794..1797) |
| | /label=″BsaI″ |
| | /ApEinfo_revcolor=#b1tt67 |
| | /ApEinfo_fwdcolor=#b1ff67 |
| terminator | comp1e1nent(1807..1915) |
| | /label=″CamR Terminator″ |
| | /ApEinfo_revcolor=#84b0dc |
| | /ApEinfo_fwdcolor=#84b0dc |
| promoter | complement(661..765) |
| | /label=″CamR Promoter″ |
| | /ApEinfo_revcolor=#84b0dc |
| | /ApEinfo_fwdcolor=#84b0dc |
| protein_bind | complement(1799..1804) |
| | /label=″BsaI″ |
| | /ApEinfo_revcolor=#b1ff67 |
| | /ApEinfo_fwdcolor=#b1ff67 |
| misc_feature | 1544..1563 |
| | /label=″Csy4″ |
| | /ApEinfo_revcolor=#f58a5e |
| | /ApEinfo_fwdcolor=#f58a5e |
| rep_origin | complement(766..1529) |
| | /label=″ColEl″ |
| | /ApEinfo_revcolor=#7f7f7f |
| | /ApEinfo_fwdcolor=#7f7f7f |
| CDS | complement(1..660) |
| | /label=″CamR″ |
| | /ApEinfo_revcolor=#0000ff |
| | /ApEinfo_fwdcolor=#0000ff |

ORIGIN

| | |
|---|---|
| 1 | ttacgccccg ccctgccact catcgcagta ctgttgtaat tcattaagca |
| | ttctgccgac |
| 61 | atggaagcca tcacaaacgg catgatgaac ctgaatcgcc agcggcatca |
| | gcaccttgtc |
| 121 | gccttgcgta taatatttgc ccatggtgaa aacgggggcg aagaagttgt |
| | ccatattggc |
| 181 | cacgtttaaa tcaaaactgg tgaaactcac ccagggattg gctgaaacga |
| | aaaacatatt |
| 241 | ctcaataaac cctttaggga aataggccag gttttcaccg taacacgcca |
| | catcttgcga |
| 301 | atatatgtgt agaaactgcc ggaaatcgtc gtggtattca ctccagagcg |
| | atgaaaacgt |
| 361 | ttcagtttgc tcatggaaaa cggtgtaaca agggtgaaca ctatcccata |
| | tcaccagctc |
| 421 | accgtctttc attgccatac gaaattccgg atgagcattc atcaggcggg |
| | caagaatgtg |
| 481 | aataaaggcc ggataaaact tgtgcttatt tttctttacg gtctttaaaa |
| | aggccgtaat |
| 541 | atccagctga acggtctggt tataggtaca ttgagcaact gactgaaatg |
| | cctcaaaatg |
| 601 | ttctttacga tgccattggg atatatcaac ggtggtatat ccagtgattt |
| | ttttctccat |
| 661 | tttagcttcc ttagctcctg aaaatctcga taactcaaaa aatacgcccg |
| | gtagtgatct |
| 721 | tatttcatta tggtgaaagt tggaacctct tacgtgcccg atcaatcatg |
| | accaaaatcc |
| 781 | cttaacgtga gttttcgttc cactgagcgt cagaccccgt agaaaagatc |
| | aaaggatctt |
| 841 | cttgagatcc tttttttctg cgcgtaatct gctgcttgca aacaaaaaaa |
| | ccaccgctac |
| 901 | cagcggtggt ttgtttgccg gatcaagagc taccaactct ttttccgaag |
| | gtaactggct |
| 961 | tcagcagagc gcagatacca aatactgttc ttctagtgta gccgtagtta |
| | ggccaccact |
| 1021 | tcaagaactc tgtagcaccg cctacatacc tcgctctgct aatcctgtta |
| | ccagtggctg |
| 1081 | ctgccagtgg cgataagtcg tgtcttaccg ggttggactc aagacgatag |
| | ttaccggata |
| 1141 | aggcgcagcg gtcgggctga acggggggtt cgtgcacaca gcccagcttg |
| | gagcgaacga |
| 1201 | cctacaccga actgagatac ctacagcgtg agctatgaga aagcgccacg |
| | cttcccgaag |
| 1261 | ggagaaaggc ggacaggtat ccggtaagcg gcagggtcgg aacaggagag |
| | cgcacgaggg |
| 1321 | agcttccagg gggaaacgcc tggtatcttt atagtcctgt cgggtttcgc |
| | cacctctgac |
| 1381 | ttgagcgtcg atttttgtga tgctcgtcag gggggcggag cctatggaaa |
| | aacgccagca |
| 1441 | acgcggcctt tttacggttc ctggcctttt gctggccttt tgctcacatg |
| | ttctttcctg |
| 1501 | cgttatcccc tgattctgtg gataaccgta tcggtctcaT GCCgttcact |
| | gccgtatagg |
| 1561 | cagctcgaga taaagcaatc ttgatgagga taatgatttt tttttgaata |
| | tacataaata |
| 1621 | ctaccgtttt tctgctagat tttgtgatga cgtaaataag tacatattac |
| | tttttaagcc |
| 1681 | aagacaagat taagcattaa ctttaccctt ttctttctaa gtttcaatat |
| | tagttatcac |
| 1741 | tgtttaaaag ttatggcgag aacgtcggcg gttaaaatat attaccctga |
| | acggctgtga |
| 1801 | gaccagacca ataaaaaacg cccggcggca accgagcgtt ctgaacaaat |
| | ccagatggag |
| 1861 | ttctgaggtc attactggat ctatcaacag gagtccaagc gagctcgata |
| | tcaaa |
| | // |

[SEQ ID NO: 82] LOCUS pLS071_-_tTDH1_[4]_modi 7915 bp ds-
DNA circular 21 JUN. 2019

| DEFINITION . | ″E. coli Marker: CamR″ |
|---|---|
| KEYWORDS | ″Seguence Verified″ ″Type: 4″ |
| FEATURES | Location/Qualifiers |
| promoter | complement(511..615) |
| | /label=″CamR Promoter″ |
| | /ApEinfo_revcolor=#84b0dc |
| | /ApEinfo_fwdcolor=#84b0dc |
| protein_bind | complement(1644..1647) |
| | /label=″BsaI″ |
| | /ApEinfo_revcolor=#b1ff67 |
| | /ApEinfo_fwdcolor=#b1ff67 |
| rep_origin | complement(616..1379) |
| | /label=″ColE1″ |
| | /ApEinfo_revcolor=#7f7f7f |
| | /ApEinfo_fwdcolor=#7f7f7f |
| misc_feature | 1394..1413 |
| | /label=″Csy4″ |
| | /ApEinfo_revcolor=#f58a5e |
| | /ApEinfo_fwdcolor=#f58a5e |
| protein_bind | complement(1649..1654) |
| | /label=″BsaI″ |
| | /ApEinfo_revcolor=#b1ff67 |
| | /ApEinfo_fwdcolor=#b1ff67 |
| CDS | complement(join(1766..1915,1..510)) |
| | /label=″CamR″ |
| | /ApEinfo_revcolor=#0000ff |
| | /ApEinfo_fwdcolor=#0000ff |
| terminator | 1420..1643 |
| | /label=″ScTDH1 Terminator″ |
| | /ApEinfo_revcolor=#ff9ccd |
| | /ApEinfo_fwdcolor=#ff9ccd |
| terminator | complement(1657..1765) |
| | /label=″CamR Terminator″ |
| | /ApEinfo_revcolor=#84b0dc |
| | /ApEinfo_fwdcolor=#84b0dc |

ORIGIN

| | |
|---|---|
| 1 | aacgggggcg aagaagttgt ccatattggc cacgtttaaa tcaaaactgg |
| | tgaaactcac |
| 61 | ccagggattg gctgaaacga aaaacatatt ctcaataaac cctttaggga |
| | aataggccag |
| 121 | gttttcaccg taacacgcca catcttgcga atatatgtgt agaaactgcc |
| | ggaaatcgtc |
| 181 | gtggtattca ctccagagcg atgaaaacgt ttcagtttgc tcatggaaaa |
| | cggtgtaaca |
| 241 | agggtgaaca ctatcccata tcaccagctc accgtctttc attgccatac |
| | gaaattccgg |
| 301 | atgagcattc atcaggcggg caagaatgtg aataaaggcc ggataaaact |
| | tgtgcttatt |
| 361 | tttctttacg gtctttaaaa aggccgtaat atccagctga acggtctggt |
| | tataggtaca |
| 421 | ttgagcaact gactgaaatg cctcaaaatg ttctttacga tgccattggg |
| | atatatcaac |
| 481 | ggtggtatat ccagtgattt ttttctccat tttagcttcc ttagctcctg |
| | aaaatctcga |
| 541 | taactcaaaa aatacgcccg gtagtgatct tatttcatta tggtgaaagt |
| | tggaacctct |
| 601 | tacgtgcccg atcaatcatg accaaaatcc cttaacgtga gttttcgttc |
| | cactgagcgt |
| 661 | cagaccccgt agaaaagatc aaaggatctt cttgagatcc tttttttctg |
| | cgcgtaatct |
| 721 | gctgcttgca aacaaaaaaa ccaccgctac cagcggtggt ttgtttgccg |
| | gatcaagagc |
| 781 | taccaactct ttttccgaag gtaactggct tcagcagagc gcagatacca |
| | aatactgttc |
| 841 | ttctagtgta gccgtagtta ggccaccact tcaagaactc tgtagcaccg |
| | cctacatacc |
| 901 | tcgctctgct aatcctgtta ccagtggctg ctgccagtgg cgataagtcg |
| | tgtcttaccg |
| 961 | ggttggactc aagacgatag ttaccggata aggcgcagcg gtcgggctga |
| | acggggggtt |
| 1021 | cgtgcacaca gcccagctcg gagcgaacga cctacaccga actgagatac |
| | ctacagcgtg |
| 1081 | agctatgaga aagcgccacg cttcccgaag ggagaaaggc ggacaggtat |
| | ccggtaagcg |
| 1141 | gcagggtcgg aacaggagag cgcacgaggg agcttccagg gggaaacgcc |
| | tggtatcttt |
| 1201 | atagtcctgt cgggtttcgc cacctctgac ttgagcgtcg atttttgtga |
| | tgatcgtcag |
| 1261 | gggggcggag cctatggaaa aacgccagca acgcggcctt tttacggttc |
| | ctggcctttt |
| 1321 | gctggccttt tgctcacatg ttctttcctg cgttatcccc tgattctgtg |
| | gataaccgta |
| 1381 | tcggtctcaC TAAgttcact gccgtatagg cagctcgaga taaagcaatc |
| | ttgatgagga |
| 1441 | taatgatttt tttttgaata tacataaata ctaccgtttt tctgctagat |
| | tttgtgatga |
| 1501 | cgtaaataag tacatattac tttttaagcc aagacaagat taagcattaa |
| | ctttaccctt |
| 1561 | ttctttctaa gtttcaatat tagttatcac tgtttaaaag ttatggcgag |
| | aacgtcggcg |
| 1621 | gttaaaatat attaccctga acggctgtga gaccagacca ataaaaaacg |
| | ccaggcggca |
| 1681 | accgagcgtt ctgaacaaat ccagatggag ttctgaggtc attactggat |
| | ctatcaacag |
| 1741 | gagtccaagc gagctcgata tcaaattacg ccccgccctg ccactcatcg |
| | cagtactgtt |
| 1801 | gtaattcatt aagcattctg ccgacatgga agccatcaca aacggcatga |
| | tgaacctgaa |
| 1861 | tcgccagcgg catcagcacc ttgtcgcctt gcgtataata tttgcccatg |
| | gtgaa |
| | // |

[SEQ ID NO: 83] LOCUS pL,S072_-_tTDH1_[4]_modi 1915 bp ds-
DNA circular 21 JUN. 2019

| DEFINITION . | ″E. coli Marker: CamR″ |
|---|---|
| KEYWORDS | ″Seguence Verified″ ″Type: 4″ |
| FEATURES | Location/Qualifiers |
| rep_origin | complement(636..1399) |
| | /label=″ColE1″ |
| | /ApEinfo_revcolor=#7f7f7f |
| | /ApEinfo_fwdcolor=#7f7f7f |
| CDS | complement(join(1786..1915,1..530)) |
| | /label=″CamR″ |
| | /ApEinfo_revcolor=#0000ff |
| | /ApEinfo_fwdcolor=#0000ff |
| misc_feature | 1414..1433 |
| | /label=″Csy4″ |
| | /ApEinfo_revcolor=#f58a5e |
| | /ApEinfo_fwdcolor=#f58aSe |
| protein_bind | complement(1664..1667) |
| | /label=″BsaI″ |
| | /ApEinfo_revcolor=#b1ff67 |
| | /ApEinfo_fwdcolor=#b1ff67 |
| protein_bind | complement(1669..1674) |
| | label=″BsaI″ |
| | /ApEinfo_revcolor=#b1ff67 |
| | /ApEinfo_fwdcolor=#b1ff67 |
| promoter | complement(531..635) |
| | /label=″CamR Promoter″ |
| | /ApEinfo_revcolor=#84b0dc |
| | /ApEinfo_fwdcolor=#84b0dc |
| terminator | 1440..1663 |
| | /label=″ScTDH1 Terminator″ |
| | /ApEinfo_revcolor=#ff9ccd |
| | /ApEinfo_fwdcolor=#ff9ccd |
| termlnator | complement(1677..1785) |
| | /label=″CamR Terminator″ |
| | /ApEinfo_revcolor=#84b0dc |
| | /ApEinfo_fwdcolor=#84b0dc |

ORIGIN

| | |
|---|---|
| 1 | taatatttgc ccatggtgaa aacgggggcg aagaagttgt ccatattggc |
| | cacgtttaaa |
| 61 | tcaaaactgg tgaaactcac ccagggattg gctgaaacga aaaacatatt |
| | ctcaataaac |
| 121 | cctttaggga aataggccag gttttcaccg taacacgcca catcttgcga |
| | atatatgtgt |
| 181 | agaaactgcc ggaaatcgtc gtggtattca ctccagagcg atgaaaacgt |
| | ttcagtttgc |
| 241 | tcatggaaaa cggtgtaaca agggtgaaca ctatcccata tcaccagctc |
| | accgtctttc |
| 301 | attgccatac gaaattccgg atgagcattc atcaggcggg caagaatgtg |
| | aataaaggcc |
| 361 | ggataaaact tgtgcttatt tttctttacg gtctttaaaa aggccgtaat |
| | atccagctga |
| 421 | acggtctggt tataggtaca ttgagcaact gactgaaatg cctcaaactg |
| | ttatttacga |
| 481 | tgccattggg atatatcaac ggtggtatat ccagtgattt ttttctccat |
| | tatttcatta |
| 541 | ttagctcctg aaaatctcga taactcaaaa aatacgcccg gtagtgatct |
| | tatttcatta |
| 601 | tggtgaaagt tggaacctct tacgtgcccg atcaatcatg accaaaatcc |
| | cttaacgtga |
| 661 | gttttcgttc cactgagcgt cagaccccgt agaaaagatc aaaggatctt |
| | cttgagatcc |
| 721 | tttttttctg cgcgtaatct gctgcttgca aacaaaaaaa ccaccgctac |
| | cagcggtggt |
| 781 | ttgtttgccg gatcaagagc taccaactct ttttccgaag gtaactggct |
| | tcagcagagc |
| 841 | gcagatacca aatactgttc ttctagtgta gccgtagtta ggccaccact |
| | tcaagaactc |
| 901 | tgtagcaccg cctacatacc tcgctctgct aatcctgtta ccagtggctg |
| | ctgccagtgg |
| 961 | cgataagtcg tgtcttaccg ggttggactc aagacgatag ttaccggata |
| | aggcgcagcg |
| 1021 | gtcgggctga acggggggtt cgtgcacaca gcccagcttg gagcgaacga |
| | cctacaccga |
| 1081 | actgagatac ctacagcgtg agctatgaga aagcgccacg cttcccgaag |
| | ggagaaaggc |
| 1141 | ggacaggtat ccggtaagcg gcagggtcgg aacaggagag cgcacgaggg |
| | agcttccagg |
| 1201 | gggaaacgcc tggtatcttt atagtcctgt cgggtttcgc cacctctgac |
| | ttgagcgtcg |
| 1261 | atttttgtga tgctcgtcag gggggcggag cctatggaaa aacgccagca |
| | acgcggcctt |
| 1321 | tttacggttc ctggcctttt gctggccttt tgctcacatg ttctttcctg |
| | cgttatcccc |
| 1381 | tgattctgtg gataaccgta tcggtctcaA CCAgttcact gccgtatagg |
| | cagctcgaga |
| 1441 | taaagcaatc ttgatgagga taatgatttt tttttgaata tacataaata |
| | ctaccgtttt |
| 1501 | tctgctagat tttgtgatga cgtaaataag tacatattac tttttaagcc |
| | aagacaagat |
| 1561 | taagcattaa ctttaccctt ttctttctaa gtttcaatat tagttatcac |
| | tgtttaaaag |
| 1621 | ttatggcgag aacgtcggcg gttaaaatat attaccctga acggctgtga |
| | gaccagacca |
| 1681 | ataaaaaacg cccggcggca accgagcgtt ctgaacaaat ccagatggag |
| | ttctgaggtc |
| 1741 | attactggat ctatcaacag gagtccaagc gagctcgata tcaaattacg |
| | ccccgccctg |
| 1801 | ccactcatcg cagtactgtt gtaattcatt aagcattctg ccgacatgga |
| | agccatcaca |
| 1861 | aacggcatga tgaacctgaa tcgccagcgg catcagcacc ttgtcgoctt |
| | gcgta |
| | // |

[SEQ ID NO: 84] LOCUS pLS073_-_tTDH1[4]_modi 1915 bp ds-
DNA circular 26 JUN. 2019

| DEFINITION . | ″E. coli Marker: CamR″ |
|---|---|
| KEYWORDS | ″Seguence Verified″ ″Type: 4″ |
| FEATURES | Location/Oualifers |
| promoter | complement(320..424) |
| | /label=″CamR Promoter″ |
| | /ApEinfo_revcolor=#84b0dc |
| | /ApEinfo_fwdcolor=#84b0dc |
| CDS | complement(join(1575..1915,1..319)) |
| | /label=″CamR″ |
| | /ApEinfo_revcolor=#0000ff |
| | /ApEinfo_fwdcolor=#0000ff |
| rep_origin | complement(425..1188) |
| | /label=″ColEl″ |
| | /ApEinfo_revcolor=#7f7f7f |
| | /ApEinfo_fwdcolor=#7f7f7f |
| misc_feature | 1203..1222 |
| | /label=″Csy4″ |
| | /ApEinfo_revcolor=#f58a5e |
| | /ApEinfo_fwdcolor=#f58a5e |
| protein_bind | complement(1453..1456) |
| | /label=″BsaI″ |
| | /ApEinfo_revcolor=#b1ff67 |
| | /ApEinfo_fwdcolor=#b1ff67 |
| terminator | 1229..1452 |
| | /label=″ScTDH1 Terminator″ |
| | /ApEinfo_revcolor=#ff9ccd |
| | /ApEinfo_fwdcolor=#ff9ccd |
| protein_bind | complement(1458..1463) |
| | /label=″BsaI″ |
| | /ApEinfo_revcolor=#b1ff67 |
| | /ApEinfo_fwdcolor=#b1ff67 |
| terminator | complement(1466..1574) |
| | /label=″CamR Terminator″ |
| | /ApEinfo_revcolor=#84b0dc |
| | /ApEinfo_fwdcolor=#84b0dc |

ORIGIN

| | |
|---|---|
| 1 | tccagagcga tgaaaacgtt tcagtttgct catggaaaac ggtgtaacaa |
| | gggtgaacac |
| 61 | tatcccatat caccagctca ccgtctttca ttgccatacg aaattccgga |
| | tgagcattca |
| 121 | tcaggcgggc aagaatgtga ataaaggccg gataaaactt gtgcttattt |
| | ttctttacgg |
| 181 | tctttaaaaa ggccgtaata tccagctgaa cggtctggtt ataggtacat |
| | tgagcaactg |
| 241 | actgaaatgc ctcaaaatgt tctttacgat gccattggga tatatcaacg |
| | gtggtatatc |
| 301 | cagtgatttt tttctccatt ttagcttcct tagctcctga aaatctcgat |
| | aactcaaaaa |
| 361 | atacgcccgg tagtgatctt atttcattat ggtgaaagtt ggaacctctt |
| | acgtgcccga |
| 421 | tcaatcatga ccaaaatccc ttaacgtgag ttttcgttcc actgagcgtc |
| | agaccccgta |
| 481 | gaaaagatca aaggatcttc ttgagatcct ttttttctgc gcgtaatctg |
| | ctgcttgcaa |
| 541 | acaaaaaaac caccgctacc agcggtggtt tgtttgccgg atcaagagct |
| | accaactctt |
| 601 | tttccgaagg taactggctt cagcagagcg cagataccaa atactgttct |
| | tctagtgtag |
| 661 | ccgtagttag gccaccactt caagaactct gtagcaccgc ctacatacct |
| | cgctctgcta |
| 721 | atcctgttac cagtggctgc tgccagtggc gataagtcgt gtcttaccgg |
| | gttggactca |
| 781 | agacgatagt taccggataa ggcgcagcgg tcgggctgaa cggggggttc |
| | gtgcacacag |
| 841 | cccagcttgg agcgaacgac ctacaccgaa ctgagatacc tacagcgtga |
| | gctatgagaa |
| 901 | agcgccacgc ttcccgdagg gagaaaggcg gacaggtatc cggtaagcgg |
| | cagggtcgga |
| 961 | acaggagagc gcacgaggga gcttccaggg ggaaacgcct ggtatcttta |
| | tagtcctgtc |
| 1021 | gggtttcgcc acctctgact tgagcgtcga tttttgtgat gctcgtcagg |
| | ggggcggagc |
| 1081 | ctatggaaaa acgccagcaa cgcggccttt ttacggttcc tggccttttg |
| | ctggcctttt |
| 1141 | gctcacatgt tctttcctgc gttatcccct gattctgtgg ataaccgtat |
| | cggtctcaAT |
| 1201 | CCgttcactg ccgtataggc agctcgagat aaagcaatct tgatgaggat |
| | aatgattttt |
| 1261 | ttttgaatat acataaatac taccgttttt ctgctagatt ttgtgatgac |
| | gtaaataagt |
| 1321 | acatattact ttttaagcca agacaagatt aagcattaac tttacccttt |
| | tctttctaag |
| 1381 | tttcaatatt agttatcact gtttaaaagt tatggcgaga acgtcggcgg |
| | ttaaaatata |
| 1441 | ttaccctgaa cggctgtgag accagaccaa taaaaaacgc ccggcggcaa |
| | ccgagcgttc |
| 1501 | tgaacaaatc cagatggagt tctgaggtca ttactggatc tatcaacagg |
| | agtccaagcg |
| 1561 | agctcgatat caaattacgc cccgccctgc cactcatcgc agtactgttg |
| | taattcatta |
| 1621 | agcattctgc cgacatggaa gccatcacaa acggcatgat gaacctgaat |
| | cgccagcggc |
| 1681 | atcagcacct tgtcgccttg cgtataatat ttgcccatgg tgaaaacggg |
| | ggcgaaccag |
| 1741 | ttgtccatat tggccacgtt taaatcaaaa ctggtgaaac tcacccaggg |
| | attggctgaa |
| 1801 | acgaaaaaca tattctcaat aaacccttta gggaaatagg ccaggttttc |
| | accgtaacac |
| 1861 | gccacatctt gcgaatatat gtgtagaaac tgccggaaat cgtcgtggta |
| | ttcac |
| | // |

The invention also provides the following numbered embodiments:

1. A method for producing an RNA mediated gene regulating or editing nucleic acid construct that comprises at least two sequences that are transcribed into nucleic acid polymers that each separately direct RNA mediated gene regulation or editing
wherein the at least two nucleic acid sequences are transcribed into a single transcript from a single promoter, wherein the method comprises:
a) amplifying a cassette from a gene regulating RNA generating (GRRG) vector using at least two GRRG primer pairs, each GRRG primer pair comprising a forward and a reverse primer,

wherein the GRRG vector comprises a selectable marker nucleic acid sequence and a nucleic acid sequence that when in RNA form comprises a cleavage site, optionally wherein the cleavage site is selected from:

i) an endoribonuclease cleavage site, for example a site-specific RNA endonuclease site, for example an artificial site-specific RNA endonucleases or a Csy4 cleavage sequence

ii) a tRNA sequence

iii) a ribozyme sequence

iv) an intron

v) a target sequence for an RNA directed cleavage complex

wherein the forward and reverse GRRG primers comprise nucleic acid sequences that are complementary to sequences of the GRRG and allow hybridisation of the primers to the GRRG vector at either side of the selectable marker sequence such that upon hybridisation the primers are directed away from the selectable marker nucleic acid sequence,

wherein the reverse GRRG primer hybridises to a common portion of the sequence that when in RNA form comprises a cleavage site, optionally wherein the sequence of the reverse primer is the same for each reverse primer in each primer pair, and wherein the forward GRRG primer hybridises to a common forward primer hybridisation sequence of the GRRG vector,

wherein the forward GRRG primer of each primer pair further comprises a sequence that encodes an RNA polymer that directs RNA mediated gene regulation or editing,

which is not complementary to the vector nucleic acid sequence and which is located 5′ of the forward primer sequence that is complementary to the GRRG

wherein amplification using each of the forward and reverse GRRG primer pairs results in the production of a linear cassette that comprises the following components in the following order 5′ to 3′:

i) the sequence that encodes an RNA polymer that directs RNA mediated gene regulation or editing ii) the forward primer hybridisation sequence

iii) the nucleic acid sequence that when in RNA form comprises a cleavage site

but which does not comprise the marker nucleic acid sequence,

optionally wherein the linear cassette comprises intervening nucleic acid located between (ii) the forward primer hybridisation sequence and (iii) the nucleic acid sequence that when in RNA form comprises a cleavage site

b) separately circularising each of the linear cassettes produced in step (a) to produce a circular nucleic acid polymer such that the sequence that encodes an RNA polymer that directs RNA mediated gene regulation or editing, is located between the forward primer hybridisation sequence and the nucleic acid sequence that when in RNA form comprises a cleavage site, optionally wherein the circularising comprises ligation of the two ends the linear cassette
c) providing at least two linking primer pairs, each primer pair comprising

a forward linking primer and a reverse linking primer,

wherein the forward linking primer is capable of hybridising to the nucleic acid sequence that when in RNA form comprises a cleavage site and the reverse linking primer is capable of hybridising to the common forward primer hybridisation sequence of the GRRG vector,

wherein each of the forward and reverse linking primers comprises a nucleic acid sequence capable of forming a single-stranded overhang, optionally wherein each primer comprises a Type II S restriction site or homing endonuclease site, wherein each pair of forward and reverse linking primers are designed so that following amplification the single-stranded overhang generated at one end of the amplification product generated by a first linking primer pair is able to hybridise with a compatible single-stranded overhang generated at one end of a second amplification product generated by a second linking primer pair;

d) amplifying each of the cassettes formed in step (b) with the appropriate pair of linking primers of (c),
e) treating the amplification products of (d) to generate a single-stranded overhang, optionally digesting the amplification products with an appropriate Type II S restriction enzyme(s) or homing endonuclease(s)
f) assembling the treated amplification products of (e) to one another to generate a single nucleic acid assembly comprising the assembled amplification products
g) ligating the single nucleic acid of (f) to a nucleic acid comprising a promoter sequence and optionally a terminator sequence,

optionally wherein the promoter nucleic acid sequence and/or optional terminator sequence has compatible overhangs to the ends of the single nucleic acid of (f), such that the promoter is located 5′ to the ligated amplification products of (f) and is capable of driving expression of a single transcript from the ligated amplification products and the optional terminator is located 3′ to the ligated amplification products of (f)

optionally where steps (f) and (g) are performed simultaneously.
2. The method of embodiment 1 wherein the sequence of the portion of the GRRG forward primer that is complementary to a sequence of the GRRG and that allows hybridisation of the primer to the GRRG vector in step (a) is the same for each forward primer of each primer pair and/or
wherein the sequence of the GRRG reverse primer that is complementary to a sequence of the GRRG and that allows hybridisation of the primer to the GRRG vector in step (a) is the same for each reverse primer of each primer pair.
3. The method of any of embodiments 1-2 wherein the promoter in step (g) is located in a destination vector and the ligation of step (g) results in the incorporation of the single nucleic acid of (f) that comprises the amplification products of (d) into the destination vector under the control of the promoter.
4. The method of any of embodiments 1-3 wherein at least two sequences that are transcribed into nucleic acid polymers that each separately direct RNA mediated gene regulation or editing are suitable for use in any one or more of CRISPR, sense Suppression/Cosuppression, antisense suppression, double-stranded RNA interference, hairpin RNA interference, intron-containing hairpin RNA interference, siRNA, micro RNA, piRNA and snoRNA.
5. The method of any of embodiments 1-4 wherein the nucleic acid construct comprises between 3 and 100 nucleic acid sequences that are transcribed into nucleic acid polymers that each separately direct RNA mediated gene regulation or editing, wherein the between 3 and 100 nucleic acid polymers are expressed as a single transcript from a single promoter, optionally wherein the nucleic acid construct comprises between and 95, 10 and 90, 15 and 85, 20 and 80, 25 and 75, 30 and 70, 35 and 65, 40 and 60, and 55 nucleic acid polymers that are transcribed into nucleic acid polymers that each separately direct RNA mediated gene regulation or editing:
optionally at least 6, 7, 8, 9, 10, 11, 12, 13, 14, 15, 16, 17, 18, 19, or at least 20 nucleic acid sequences that are transcribed into nucleic acid polymers that each separately direct RNA mediated gene regulation or editing, optionally at least 11 or at least 12 nucleic acid sequences that are transcribed into nucleic acid polymers that each separately direct RNA mediated gene regulation or editing.
6. The method of any of embodiments 1-5 wherein the promoter of (g) is:
a) a Pol II promoter, optionally

wherein the Pol II promoter is classed as a strong promoter:

wherein the promoter is an inducible promoter; and/or

wherein the promoter is selected from the group consisting of TDH3 promoter, TEF1 promoter, PGK1 promoter, pCCW12 promoter, pTEF2 promoter, pHHF1 promoter, pHHF2 promoter, pALD6 promoter, pGal1 promoter (galactose-inducible), pPGK1 promoter, pHTB2 promoter or pCUP1 promoter (induced by copper-sulfate), or a tetracycline-inducible promoter; or

b) a Pol III promoter, optionally

wherein the Pol III promoter is classed as a strong Po 111I promoter;

wherein the Po III promoter is an inducible promoter; and/or

wherein the Pol III is selected from the group consisting of the tRNA Phe promoter with a 5′ HDV ribozyme, the U6 promoter or the H1 promoter.

7. The method of any of embodiments 1-6 wherein the sequence of the GRRG to which the forward GRRG primer hybridises does not form part of the nucleic acid that directs RNA mediated gene regulation or editing.
8. The method of any of embodiments 1-6 wherein the sequence of the GRRG to which the forward GRRG primer hybridises encodes part of the nucleic acid that directs RNA mediated gene regulation or editing.
9. The method of any of embodiments 1-8 wherein the GGRG vector comprises a scaffold sequence that when in RNA form allows association of the RNA with a polypeptide capable of regulating or editing a gene, optionally wherein the polypeptide is selected from the group consisting of:

Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

10. The method of embodiment 9 wherein the common forward primer hybridisation sequence of the GRRG vector sequence at least partly overlaps with the scaffold sequence.
11. The method of any of embodiments 1-10 wherein the sequence that encodes an RNA mediated gene regulation or editing directing sequence that is part of the forward primer comprises RNA for association with a Cas9 or Cas9-like protein, optionally Cas13a/C3c2 optionally comprises sgRNA sequence.
12. The method of any of embodiments 1-11 wherein the at least two nucleic acid sequences that encode an RNA mediated gene regulation or editing directing sequence(s) are directed towards different genes, optionally wherein each nucleic acid sequence that encodes an RNA mediated gene regulation or editing directing sequence is directed towards a different gene.
13. A method of producing at least two nucleic acid sequences that direct RNA mediated gene regulation or editing wherein the method comprises expressing an RNA transcript from the RNA mediated gene regulating or editing nucleic acid construct according to any of embodiments 1-12,

optionally wherein the method produces at least 11 or at least 12 nucleic acid polymers that direct RNA mediated gene regulation or editing.

14. The method of embodiment 13 wherein the RNA transcript is expressed in the presence of an agent that is capable of cleaving the sequence that when in RNA form is specifically cleavable, optionally in the presence of Csy4.
15. The method of any of embodiments 13 and 14 wherein the method further comprises transforming the RNA mediated gene regulating or editing nucleic acid construct produced by the method of any of embodiments 1-12 into a cell, optionally wherein the cell expresses or comprises or is exposed to an agent that is capable of cleaving the sequence that when in RNA form is specifically cleavable, optionally expresses or comprises or is exposed to Csy4.
16. The method of any of embodiments 13-15 wherein where at least one of the nucleic acid sequences that directs RNA mediated gene regulation or editing is a sgRNA, the method further comprises co-expressing a polypeptide capable of associating with the sgRNA, wherein the polypeptide is selected from the group consisting of:
Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida);

optionally wherein the polypeptide is fused to an activation and/or repression domain, optionally

wherein the activation domain is selected from the group consisting of VP, VP16, VP64, Gal4, or B42; and/or

wherein the repression domain is selected from the group consisting of KRAB-like effectors (e.g. Mxi1), RD1152, RD11, RD5 or RD2; or

optionally wherein the polypeptide is fused to an error prone DNA polymerase.
17. A single RNA molecule that comprises at least 2 nucleic acid sequences that are each separately capable of directing RNA mediated gene regulation or editing, wherein between each nucleic acid sequence that directs RNA mediated gene regulation or editing is a sequence that is a cleavage site, optionally wherein the cleavage site is selected from the group consisting of a Csy4 cleavage site, a tRNA sequence, a ribozyme sequence, an intron sequence, or a target sequence for an RNA directed cleavage complex

optionally wherein the single RNA molecule comprises between 11 and 100 nucleic acid sequences that direct RNA mediated gene regulation or editing, optionally 12 and 90, 13 and 80, 14 and 70, 15 and 60, 20 and 50, 30 and 40, nucleic acid sequences that direct RNA mediated gene regulation or editing,

optionally wherein the single RNA molecule comprises 11 or 12 nucleic acid sequences that direct RNA mediated gene regulation or editing,

optionally wherein the single RNA molecule has been produced by the method of any of embodiments 1-12.

18. A single nucleic acid molecule that comprises at least 2 nucleic acid sequences that encode an RNA mediated gene regulation or editing directing nucleic acid polymer, wherein between each sequence that encodes an RNA mediated gene regulation or editing directing nucleic acid polymer is a sequence that when in RNA form is a cleavage site, optionally wherein the cleavage site is selected from the group consisting of a Csy4 cleavage site, a tRNA sequence, a ribozyme sequence, an intron sequence or a target sequence for an RNA directed cleavage complex, wherein the single nucleic acid molecule comprises a promoter capable of driving expression from the at least 11 nucleic acid sequences to form one single RNA transcript,

optionally wherein the single nucleic acid molecule comprises between 11 and 100 nucleic acid sequences that encode an RNA mediated gene regulation or editing directing nucleic acid polymer, optionally 12 and 90 13 and 80, 14 and 70, 15 and 60, 20 and 50, and 40 nucleic acid sequences that encode an RNA mediated gene regulation or editing directing nucleic acid polymer,

optionally wherein the single nucleic acid molecule comprises 11 or 12 nucleic acid sequences that encode an RNA mediated gene regulation or editing nucleic acid polymer,

optionally wherein the single nucleic acid molecule has been produced by the method of any of embodiments 1-12, optionally wherein the nucleic acid is DNA.

19. A phage or viral vector comprising the single RNA molecule of embodiment 17 or the single nucleic acid molecule or any of embodiments 18, optionally wherein the phage or viral vector is selected from the group consisting of adeno-associated virus (AAV), Hybrid Adenoviral Vectors or Herpes simplex viruses.
20. A cell comprising the single RNA molecule of embodiment 17 or the single nucleic acid molecule or any of embodiments 18 or the phage vector of embodiment 19.
21. The cell of embodiment 20 wherein the cell expresses or comprises or is exposed to an agent that is capable of cleaving the sequence that when in RNA form comprises a cleavage site, optionally wherein

where the sequence that when in RNA form is a cleavage site comprises the Csy4 cleavage site, the cell expresses or comprises or is exposed to Csy4 polypeptide;

where the sequence that when in RNA form is a cleavage site comprises a tRNA sequence, the cell expresses or comprises or is exposed to RNase P, RNase Z and/or RNase E;

where the sequence that when in RNA form is a cleavage site comprises a ribozyme cleavage site, the cell expresses or comprises or is exposed to the appropriate ribozyme;

where the sequence that when in RNA form is a cleavage site comprises an intron, the cell expresses or comprises or is exposed to native splicing machinery;

22. A method for the regulation or editing of at least one gene in a cell wherein the method comprises

the method for producing an RNA mediated gene regulating or editing nucleic acid construct that comprises at least two sequences that are transcribed into nucleic acid polymers that each separately direct RNA mediated gene regulation or editing according to any of embodiments 1-12;

the method for producing at least two nucleic acid polymers that direct RNA mediated gene regulation or editing according to any of embodiments 13-16, optionally at least 11 or at least 12 nucleic acid polymers that direct RNA mediated gene regulation or editing according to any of embodiments 13-16;

the use of the nucleic acid molecule according to embodiment 17;

the use of the nucleic acid molecule according embodiment 18;

the use of the phage according to embodiment 19; and/or

the use of the cell according to embodiment 20 or 21.

23. A single nucleic acid according to any of embodiments 17 or 18, the phage according to embodiment 19, or the cell according to any of embodiments 20 or 21 for use in
a) medicine, optionally for use in the treatment and/or prevention of a disease, optionally for use as a vaccine,

optionally for the treatment or prevention of a disease in which entire pathways are dysregulated, optionally wherein the disease is selected from the group consisting of Glioblastoma multiforme, Diabetes (type I and type II), Multiple sclerosis, Autoimmune diseases and Huntington's disease; or

b) an industrial process, optionally for use in brewing, large-scale protein production, pharmaceutical production, metabolite production, optionally the production of chemicals or fuels, biomass vs. growth or metabolic ‘valves’.
24. A gene regulating RNA generating (GRRG) vector comprising a selectable marker and a nucleic acid sequence that when in RNA form comprises a cleavage site, optionally wherein the cleavage site is selected from a Csy4 cleavage site, a tRNA, a ribozyme cleavage site, an intron, or a target sequence for an RNA directed cleavage complex
25. The gene regulating RNA generating vector of embodiment 24 wherein the vector further comprises a scaffold sequence that when in RNA form allows association of the RNA with a polypeptide capable of regulating or editing a gene, optionally wherein the polypeptide is selected from the group consisting of:

Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida);

optionally wherein the polypeptide is fused to an activation and/or repression domain, optionally

wherein the activation domain is selected from the group consisting of VP, VP16, VP64, Gal4, or B42; and/or

wherein the repression domain is selected from the group consisting of KRAB-like effectors (e.g. Mxi1), RD1152, RD11, RD5 or RD2.

26. The gene regulating RNA generating vector of embodiment 25 wherein the vector comprises the following components in the following order 5′ to 3′:
a) nucleic acid sequence that when in RNA form comprises a Csy4 cleavage site, a tRNA, a ribozyme cleavage site, an intron or a target sequence for an RNA directed cleavage complex
b) the selectable marker; and
c) the scaffold sequence.
27. A kit comprising any two or more of
i) a GRRG vector according to any of embodiments 24-26 or as defined in any of the preceding embodiments
ii) a GRRG forward and reverse primer according to the invention
iii) one or more linking primer pairs according to the invention
iv) a destination vector according to the invention
v) a nucleic acid encoding a polypeptide selected from the group consisting of Cas9, optionally
wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida),
optionally wherein the polypeptide is fused to an activator or repressor domain, or an error-prone DNA polymerase
vi) a Type II S restriction enzyme, optionally BsmBI;
vii) a nucleic acid encoding a Csy4 polypeptide, optionally wherein the nucleic acid is a circular vector;
vii) one or more restriction enzymes
ix) DNA polymerase
x) DNA ligase
optionally wherein the kit comprises the GRRG vector of (i).

1. A method for producing an RNA mediated gene regulating or editing nucleic acid construct that comprises at least two sequences that are transcribed into nucleic acid polymers that each separately direct RNA mediated gene regulation or editing
wherein the at least two nucleic acid sequences are transcribed into a single transcript from a single promoter, wherein the method comprises:
a) amplifying a cassette from a gene regulating RNA generating (GRRG) vector using at least two GRRG primer pairs, each GRRG primer pair comprising a forward and a reverse primer,
wherein the GRRG vector comprises a selectable marker nucleic acid sequence and a nucleic acid sequence that when in RNA form comprises a cleavage site
wherein the forward and reverse GRRG primers comprise nucleic acid sequences that are complementary to sequences of the GRRG and allow hybridisation of the primers to the GRRG vector at either side of the selectable marker sequence such that upon hybridisation the primers are directed away from the selectable marker nucleic acid sequence,
wherein the reverse GRRG primer hybridises to a common portion of the sequence that when in RNA form comprises a cleavage site,
wherein the forward GRRG primer of each primer pair further comprises a sequence that encodes an RNA polymer that directs RNA mediated gene regulation or editing, which is not complementary to the vector nucleic acid sequence and which is located 5′ of the forward primer sequence that is complementary to the GRRG
wherein amplification using each of the forward and reverse GRRG primer pairs results in the production of a linear cassette that comprises the following components in the following order 5′ to 3′:
i) the sequence that encodes an RNA polymer that directs RNA mediated gene regulation or editing
ii) the forward primer hybridisation sequence
iii) the nucleic acid sequence that when in RNA form comprises a cleavage site but which does not comprise the marker nucleic acid sequence; and
b) separately circularising each of the linear cassettes produced in step (a) to produce a circular nucleic acid polymer such that the sequence that encodes an RNA polymer that directs RNA mediated gene regulation or editing, is located between the forward primer hybridisation sequence and the nucleic acid sequence that when in RNA form comprises a cleavage site; and
c) providing at least two linking primer pairs, each primer pair comprising
a forward linking primer and a reverse linking primer,
wherein the forward linking primer is capable of hybridising to the nucleic acid sequence that when in RNA form comprises a cleavage site and the reverse linking primer is capable of hybridising to the common forward primer hybridisation sequence of the GRRG vector,
wherein each of the forward and reverse linking primers comprises a nucleic acid sequence capable of forming a single-stranded overhang; and
d) amplifying each of the cassettes formed in step (b) with the appropriate pair of linking primers of (c); and
e) treating the amplification products of (d) to generate a single-stranded overhang; and
f) assembling the treated amplification products of (e) to one another to generate a single nucleic acid assembly comprising the assembled amplification products; and either
g) ligating the single nucleic acid of (f) to a nucleic acid destination or expression vector:
or
(h) (i) ligating the single nucleic acid of (f) to an intermediate nucleic acid vector producing an intermediate vector comprising the single nucleic acid assembly of step (f);
(ii) performing steps (a) to (f) and (h)(i) at least twice resulting in at least two different intermediate vectors each comprising a different single nucleic acid assembly of step (f);
(iii) digesting the respective at least two intermediate vectors to produce at least two cleavage fragments comprising different nucleic acid assemblies; and/or amplifying the at least two different nucleic acid assemblies from the at least two intermediate vectors;
(iv) ligating the at least two cleavage fragments or the at least two amplification products into a single destination or expression vector producing an array of nucleic acid assemblies of (f),
wherein the destination or expression vector comprises a promoter and optionally a terminator, wherein the promoter is located 5′ to the array of nucleic acid assemblies of (f) and is capable of driving expression of a single transcript from the array, and the optional terminator is located 3′ to the array of nucleic acid assemblies of (f).

2. The method according to claim 1 wherein the cleavage site of the GRRG vector is selected from:
i) an endoribonuclease cleavage site, for example a site-specific RNA endonuclease site, for example a Csy4 cleavage sequence or an artificial site-specific RNA endonucleases or
ii) a tRNA sequence
iii) a ribozyme sequence
iv) an intron
v) a target sequence for an RNA directed cleavage complex

3. The method according to any of claims 1 or 2 wherein the sequence of the reverse GRRG primer is the same for each reverse primer in each primer pair, and wherein the forward GRRG primer hybridises to a common forward primer hybridisation sequence of the GRRG vector.

4. The method according to any of claims 1-3 wherein the linear cassette of step (a) comprises intervening nucleic acid located between (ii) the forward primer hybridisation sequence and (ii) the nucleic acid sequence that when in RNA form comprises a cleavage site.

5. The method according to any of claims 1-4 wherein the circularising of step (b) comprises ligation of the two ends the linear cassette.

6. The method according to any of claims 1-5 wherein the sequence capable of forming a single-stranded overhang of the forward and reverse linking primers of step (c) is a Type II S restriction site or homing endonuclease site, wherein each pair of forward and reverse linking primers are designed so that following amplification the single-stranded overhang generated at one end of the amplification product generated by a first linking primer pair is able to hybridise with a compatible single-stranded overhang generated at one end of a second amplification product generated by a second linking primer pair.

7. The method according to any of claims 1-6 wherein said treating of step (e) involves digesting the amplification products with an appropriate Type II S restriction enzyme(s) or homing endonuclease(s).

8. The method according to any of claims 1-7 wherein the destination or expression vector of (g) or (h)(iv) comprises a promoter sequence, and optionally a terminator sequence.

9. The method according to any of claims 1-8 wherein the promoter and/or terminator sequence of the destination or expression vector has compatible overhangs to the ends of the single nucleic acid of (f), such that the promoter is located 5′ to the ligated amplification products of (f) and is capable of driving expression of a single transcript from the ligated amplification products and the optional terminator is located 3′ to the ligated amplification products of (f).

10. The method according to any of claims 1-9 wherein steps (f) and (g) or (f) and (h)(i) are performed simultaneously.

11. The method of any of claims 1-10 wherein the sequence of the portion of the GRRG forward primer that is complementary to a sequence of the GRRG and that allows hybridisation of the primer to the GRRG vector in step (a) is the same for each forward primer of each primer pair and/or
wherein the sequence of the GRRG reverse primer that is complementary to a sequence of the GRRG and that allows hybridisation of the primer to the GRRG vector in step (a) is the same for each reverse primer of each primer pair.

12. The method of any of claims 1-11 wherein the ligating of step (g) results in the incorporation of the single nucleic acid of (f) that comprises the amplification products of (d) into the destination vector under the control of the promoter.

13. The method of any of claims 1-12 wherein at least two sequences that are transcribed into nucleic acid polymers that each separately direct RNA mediated gene regulation or editing are suitable for use in any one or more of CRISPR, sense Suppression/Cosuppression, antisense suppression, double-stranded RNA interference, hairpin RNA interference, intron-containing hairpin RNA interference, siRNA, micro RNA, piRNA and snoRNA.

14. The method of any of claims 1-13 wherein the nucleic acid construct comprises between 3 and 100 nucleic acid sequences that are transcribed into nucleic acid polymers that each separately direct RNA mediated gene regulation or editing, wherein the between 3 and 100 nucleic acid polymers are expressed as a single transcript from a single promoter.

15. The method of according to any of claims 1-14 wherein the nucleic acid construct comprises
between 5 and 95, 10 and 90, 15 and 85, 20 and 80, 25 and 75, 30 and 70, 35 and 65, 40 and 60, 45 and 55 nucleic acid polymers that are transcribed into nucleic acid polymers that each separately direct RNA mediated gene regulation or editing; or
at least 6, 7, 8, 9, 10, 11, 12, 13, 14, 15, 16, 17, 18, 19, or at least 20 nucleic acid sequences that are transcribed into nucleic acid polymers that each separately direct RNA mediated gene regulation or editing, optionally at least 11 or at least 12 nucleic acid sequences that are transcribed into nucleic acid polymers that each separately direct RNA mediated gene regulation or editing.

16. The method of any of claims 1-15 wherein the promoter of the destination or expression vector is:
a) a Pol II promoter, optionally
wherein the Pol II promoter is classed as a strong promoter;
wherein the promoter is an inducible promoter; and/or
wherein the promoter is selected from the group consisting of TDH3 promoter, TEF1 promoter, PGK1 promoter, pCCW12 promoter, pTEF2 promoter, pHHF1 promoter, pHHF2 promoter, pALD6 promoter, pGal1 promoter (galactose-inducible), pPGK1 promoter, pHTB2 promoter or pCUP1 promoter (induced by copper-sulfate), or a tetracycline-inducible promoter; or
b) a Pol III promoter, optionally
wherein the Pol III promoter is classed as a strong Po 111I promoter;
wherein the Pol III promoter is an inducible promoter; and/or
wherein the Pol III is selected from the group consisting of the tRNA Phe promoter with a 5′ HDV ribozyme, the U6 promoter or the H1 promoter.

17. The method of any of claims 1-16 wherein the sequence of the GRRG to which the forward GRRG primer hybridises does not form part of the nucleic acid that directs RNA mediated gene regulation or editing.

18. The method of any of claims 1-16 wherein the sequence of the GRRG to which the forward GRRG primer hybridises encodes part of the nucleic acid that directs RNA mediated gene regulation or editing.

19. The method of any of claims 1-18 wherein the GGRG vector comprises a scaffold sequence that when in RNA form allows association of the RNA with a polypeptide capable of regulating or editing a gene, optionally wherein the polypeptide is selected from the group consisting of:
Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida).

20. The method of claim 19 wherein the common forward primer hybridisation sequence of the GRRG vector sequence at least partly overlaps with the scaffold sequence.

21. The method of any of claims 1-20 wherein the sequence that encodes an RNA mediated gene regulation or editing directing sequence that is part of the forward primer comprises RNA for association with a Cas9 or Cas9-like protein, optionally Cas13a/C3c2 optionally comprises sgRNA sequence.

22. The method of any of claims 1-21 wherein the at least two nucleic acid sequences that encode an RNA mediated gene regulation or editing directing sequence(s) are directed towards different genes, optionally wherein each nucleic acid sequence that encodes an RNA mediated gene regulation or editing directing sequence is directed towards a different gene.

23. A single RNA molecule that comprises at least 2 nucleic acid sequences that are each separately capable of directing RNA mediated gene regulation or editing, wherein between each nucleic acid sequence that directs RNA mediated gene regulation or editing is a sequence that is a cleavage site.

24. The single RNA molecule according to claim 23 wherein the cleavage site is selected from the group consisting of a Csy4 cleavage site, a tRNA sequence, a ribozyme sequence, an intron sequence, or a target sequence for an RNA directed cleavage complex.

25. The single RNA molecule according to any of claims 23 or 24 wherein the single RNA molecule comprises between 11 and 100 nucleic acid sequences that direct RNA mediated gene regulation or editing, optionally
comprises between 12 and 90, 13 and 80, 14 and 70, 15 and 60, 20 and 50, 30 and 40, nucleic acid sequences that direct RNA mediated gene regulation or editing; or
comprises 11 or 12 nucleic acid sequences that direct RNA mediated gene regulation or editing.

26. The single RNA molecule according to any of claims 23-25 wherein the single RNA molecule has been produced by the method of any of claims 1-22.

27. An RNA mediated gene regulating or editing nucleic acid construct which is a single nucleic acid molecule that comprises at least 2 nucleic acid sequences that encode an RNA mediated gene regulation or editing directing nucleic acid polymer, wherein between each sequence that encodes an RNA mediated gene regulation or editing directing nucleic acid polymer is a sequence that when in RNA form is a cleavage site.

28. The RNA mediated gene regulating or editing nucleic acid construct according to claim 27 wherein the cleavage site is selected from the group consisting of a Csy4 cleavage site, a tRNA sequence, a ribozyme sequence, an intron sequence or a target sequence for an RNA directed cleavage complex.

29. The RNA mediated gene regulating or editing nucleic acid construct according to any of claims 27 or 28 wherein the single nucleic acid molecule comprises a promoter capable of driving expression from the at least 2 nucleic acid sequences to form one single RNA transcript.

30. The RNA mediated gene regulating or editing nucleic acid construct according to any of claims 27-29 wherein the single nucleic acid molecule comprises between 1 and 100 nucleic acid sequences that encode an RNA mediated gene regulation or editing directing nucleic acid polymer, optionally between 11 and 100 nucleic acid sequences that encode an RNA mediated gene regulation or editing directing nucleic acid polymer, optionally between 12 and 90 13 and 80, 14 and 70, 15 and 60, 20 and 50, 30 and 40 nucleic acid sequences that encode an RNA mediated gene regulation or editing directing nucleic acid polymer,
optionally wherein the single nucleic acid molecule comprises 11 or 12 nucleic acid sequences that encode an RNA mediated gene regulation or editing nucleic acid polymer.

31. The RNA mediated gene regulating or editing nucleic acid construct according to any of claims 27-30 wherein the single nucleic acid molecule has been produced by the method of any of claims 1-22.

32. A phage or viral vector comprising the single RNA molecule of any of claims 23-26 or the single nucleic acid molecule or any of claims 27-31, optionally wherein the phage or viral vector is selected from the group consisting of adeno-associated virus (AAV), Hybrid Adenoviral Vectors or Herpes simplex viruses.

33. A cell comprising the single RNA molecule of any of claims 23-26 or the single nucleic acid molecule or any of claims 27-31 or the phage or viral vector of claim 32.

34. The cell of claim 33 wherein the cell expresses or comprises or is exposed to an agent that is capable of cleaving the sequence that when in RNA form comprises a cleavage site, optionally wherein
where the sequence that when in RNA form is a cleavage site comprises the Csy4 cleavage site, the cell expresses or comprises or is exposed to Csy4 polypeptide;
where the sequence that when in RNA form is a cleavage site comprises a tRNA sequence, the cell expresses or comprises or is exposed to RNase P, RNase Z and/or RNase E;
where the sequence that when in RNA form is a cleavage site comprises a ribozyme cleavage site, the cell expresses or comprises or is exposed to the appropriate ribozyme;
where the sequence that when in RNA form is a cleavage site comprises an intron, the cell expresses or comprises or is exposed to native splicing machinery.

35. A method of producing at least two nucleic acid sequences that direct RNA mediated gene regulation or editing wherein the method comprises expressing an RNA transcript from the RNA mediated gene regulating or editing nucleic acid construct according to any of claims 27-31.

36. The method according to claim 35 wherein the method produces at least 11 or at least 12 nucleic acid polymers that direct RNA mediated gene regulation or editing.

37. The method of any of claims 35 or 26 wherein the RNA transcript is expressed in the presence of an agent that is capable of cleaving the sequence that when in RNA form is specifically cleavable, optionally expressed in the presence of Csy4.

38. The method of any of claims 35-37 wherein the method further comprises transforming the RNA mediated gene regulating or editing nucleic acid construct of any of claims 27-31 into a cell, optionally wherein the cell expresses or comprises or is exposed to an agent that is capable of cleaving the sequence that when in RNA form is specifically cleavable, optionally expresses or comprises or is exposed to Csy4.

39. The method of any of claims 35-38 wherein where at least one of the nucleic acid sequences that directs RNA mediated gene regulation or editing is a sgRNA, the method further comprises co-expressing a polypeptide capable of associating with the sgRNA.

40. The method according to claim 39 wherein the polypeptide capable of associating with the sgRNA is:
a) Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida); and/or
b) fused to an activation and/or repression domain, optionally
wherein the activation domain is selected from the group consisting of VP, VP16, VP64, Gal4, or B42; and/or
wherein the repression domain is selected from the group consisting of KRAB-like effectors (e.g. Mxi1), RD1152, RD11, RD5 or RD2; or
c) an error prone DNA polymerase.

41. A method for the regulation or editing of at least one gene in a cell wherein the method comprises
the method for producing an RNA mediated gene regulating or editing nucleic acid construct that comprises at least two sequences that are transcribed into nucleic acid polymers that each separately direct RNA mediated gene regulation or editing according to any of claims 1-22;
the method for producing at least two nucleic acid polymers that direct RNA mediated gene regulation or editing according to any of claims 35-40;
the use of the nucleic acid molecule according to any of claims 23-26;
the use of the RNA mediated gene regulating or editing nucleic acid construct according to any one of claims 27-31;
the use of the phage according to claim 32; and/or
the use of the cell according to claim 33 or 34.

42. A single nucleic acid according to any of claims 23 to 26, the RNA mediated gene regulating or editing nucleic acid construct according to any one of claims 27-31, the phage according to claim 32, or the cell according to any of claims 33 or 34 for use in medicine, optionally for use in the treatment and/or prevention of a disease, optionally for use as a vaccine.

43. The single nucleic acid according to any of claims 23 to 26, the RNA mediated gene regulating or editing nucleic acid construct according to any one of claims 27-31, the phage according to claim 32, or the cell according to any of claims 33 or 34 for use according to claim 42 for the treatment or prevention of a disease in which entire pathways are dysregulated, optionally wherein the disease is selected from the group consisting of Glioblastoma multiforme, Diabetes (type I and type II), Multiple sclerosis, Autoimmune diseases and Huntington's disease.

44. The single nucleic acid according to any of claims 23 to 26, the RNA mediated gene regulating or editing nucleic acid construct according to any one of claims 27-31, the phage according to claim 32, or the cell according to any of claims 33 or 34 for use in an industrial process, optionally for use in brewing, large-scale protein production, pharmaceutical production, metabolite production, optionally the production of chemicals or fuels, biomass vs. growth or metabolic ‘valves’.

45. A gene regulating RNA generating (GRRG) vector comprising a selectable marker and a nucleic acid sequence that when in RNA form comprises a cleavage site, optionally wherein the cleavage site is selected from a Csy4 cleavage site, a tRNA, a ribozyme cleavage site, an intron, or a target sequence for an RNA directed cleavage complex.

46. The gene regulating RNA generating vector of claim 45 wherein the vector further comprises a scaffold sequence that when in RNA form allows association of the RNA with a polypeptide capable of regulating or editing a gene, optionally wherein the polypeptide capable of regulating or editing a gene is:
a) Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida); and/or
b) fused to an activation and/or repression domain, optionally
wherein the activation domain is selected from the group consisting of VP, VP16, VP64, Gal4, or B42; and/or
wherein the repression domain is selected from the group consisting of KRAB-like effectors (e.g. Mxi1), RD1152, RD11, RD5 or RD2; and/or
c) an error prone DNA polymerase.

47. The gene regulating RNA generating vector according to any of claims 45 or 46 wherein the vector comprises the following components in the following order 5′ to 3′:
a) nucleic acid sequence that when in RNA form comprises a Csy4 cleavage site, a tRNA, a ribozyme cleavage site, an intron or a target sequence for an RNA directed cleavage complex
b) the selectable marker; and
c) the scaffold sequence.

48. A kit comprising any two or more of:
i) a GRRG vector according to any of claims 45-47 or as defined in any of the preceding
ii) a GRRG forward and reverse primer according to the invention
iii) one or more linking primer pairs according to the invention
iv) a destination vector according to the invention
v) a nucleic acid encoding a polypeptide capable of regulating or editing a gene, optionally wherein the polypeptide capable of regulating or editing a gene is:
a) Cas9 or Cas9-like polypeptide, optionally wherein the Cas9 polypeptide is a Streptococcus pyogenes Cas9 polypeptide; Cas12a; Cas12b; Cas13a; Cas13b; LbCpf1 (Lachnospiraceae bacterium ND2006)—most commonly used; AsCpf1 (from Acidaminococcus); or FnCpf1 (Francisella novicida); and/or
b) fused to an activation and/or repression domain, optionally wherein the activation domain is selected from the group consisting of VP, VP16, VP64, Gal4, or B42; and/or
wherein the repression domain is selected from the group consisting of KRAB-like effectors (e.g. Mxi1), RD1152, RD11, RD5 or RD2; and.or
c) an error prone DNA polymerase
vi) one or more Type II S restriction enzymes, optionally BsmBI;
vii) a nucleic acid encoding a Csy4 polypeptide, optionally wherein the nucleic acid is a circular vector;
vii) one or more restriction enzymes
ix) DNA polymerase
x) DNA ligase
xi) one or more intermediate vectors
optionally wherein the kit comprises the GRRG vector of (i).
